(19) United States
(12) Patent Application Publication
(10) Pub. No.: US 2006/0128660 A1
(43) Pub. Date: Jun. 15, 2006

(54) FK228 analogs and methods of making and using the same

(75) Inventors: Scott R. Rajski, Madison, WI (US); David A. Wassarman, Middleton, WI (US); Jose A. Restituyo, Madison, WI (US)

Correspondence Address: GODFREY & KAHN, S.C., 780 N. WATER STREET, MILWAUKEE, WI, 53202, US

(73) Assignee: Wisconsin Alumni Research Foundation, Madison, WI (US)

(21) Appl. No.: 10/905,030

(22) Filed: Dec. 10, 2004

(51) Int. Cl. A61K 31/537; A61K 31/4745; A61K 31/4747; A61K 31/473; A61K 31/695; A61K 31/44; A61K 31/47; A61K 31/353; A61K 31/265

(52) U.S. Cl. 514/63; 514/296; 514/278; 514/237.5; 514/317; 514/255.04; 514/345; 514/408; 514/309; 514/316; 514/279; 514/456; 514/512; 514/357; 514/412; 514/285

(57) ABSTRACT

The present invention provides FK228 analogs and methods of making and using the same. Such analogs are potent inhibitors of histone deacetylase and, in certain embodiments, are capable of specifically targeting cancerous cells and tissues. In preferred embodiments, these analogs are characterized by a cyclic disulfide design.

### FIELD OF INVENTION

This invention relates to the inhibition of histone deacetylase. More particularly, the invention is directed to analogs of the anti-cancer drug FK228 and methods of making and using the same.

### BACKGROUND OF THE INVENTION

The natural product FK228 (1), formally known as FR901228, is a histone deacetylase (HDAC) inhibitor possessing anti-tumor activity but very little toxicity in normal cells (Richon et al., 1998, Proc. Natl. Acad. Sci. USA, 95: 3003-3007). Currently, FK228 is in clinical studies for chromic lymphocytic leukemia, small lymphocytic lymphoma, acute myeloid leukemia, cutaneous T-cell lymphoma, and refractory small cell lung cancer. Unfortunately, chemical syntheses of FK228 have proven difficult and yields of the natural product from microbial cultures are disappointingly inadequate.

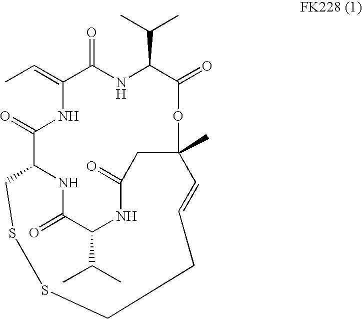

To date, many known inhibitors of histone deacetylase are known in the art, however, they are not known to have cellular selectivity. Thus, there exists a need to identify additional HDAC inhibitors as well as the structural features required for potent HDAC inhibitory activity. Analogs of FK228 that possesed the bioactivity of the parent compound while being easier and less costly to obtain are particularly desirable.

### SUMMARY OF THE INVENTION

In one aspect, the present invention provides compositions for inhibiting a histone deacetylase based upon novel structurally simple analogs of FK228. These compositions comprise a novel analog represented by the general formula: 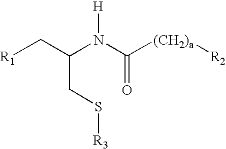
wherein R₁ is —OH, NH, NHR, tert-butyidiphenylsilyl (TBSPS—O—)

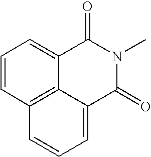

or a cap structure selected from the group consisting of unsubstituted and substituted alkyls, alkenyls, alkynyls, cycloalkyls, aryls, heterocyclyls, phthalimides, naphthalimides and polycyclic phenols;

wherein R₂ is a —SH or —SCOCH₃;

wherein R₃ is —H or a structure selected from the group consisting of unsubstituted and substituted alkyls, alkenyls, alkynyls, cycloalkyls, aryls, and heterocyclyls; and

wherein n is an integer from 1 to 7.

In a preferred composition according to the invention, the compound includes an R₁ group that is a tert-butyidiphenylsilyl (TBSPS—O—) group or

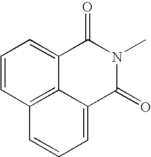

In other preferred compositions according to the invention, R₁ is a cap structure selected from the group consisting of:

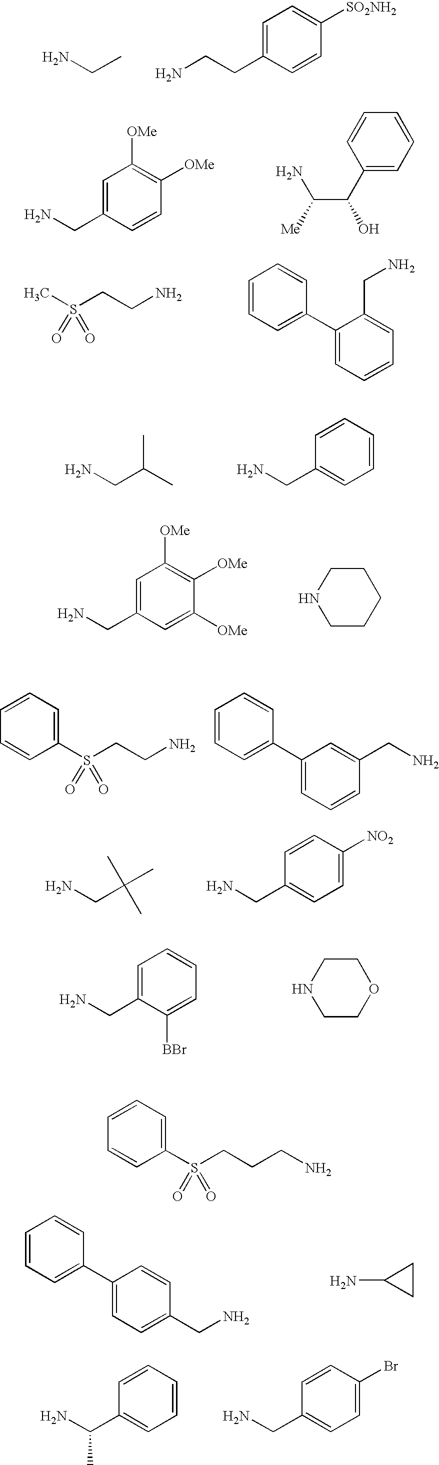

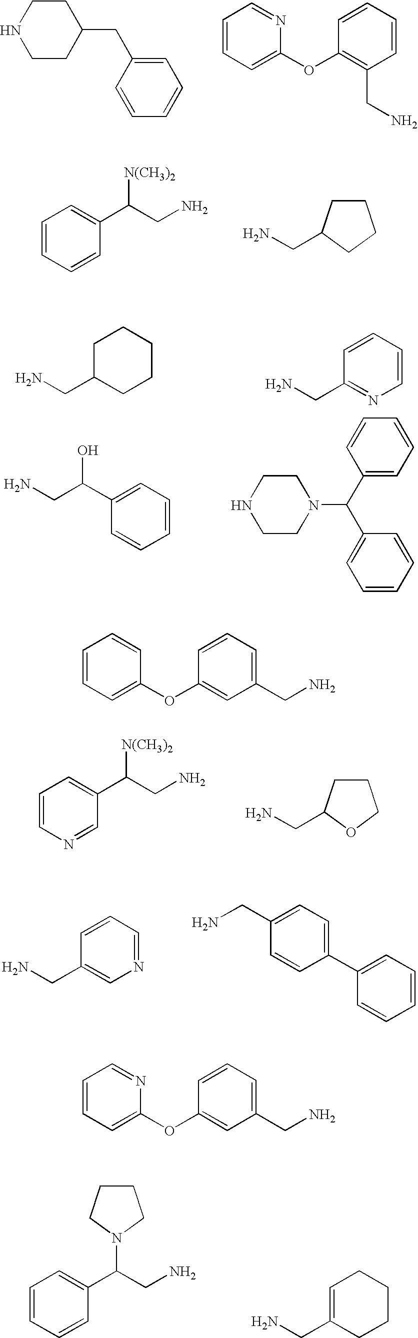

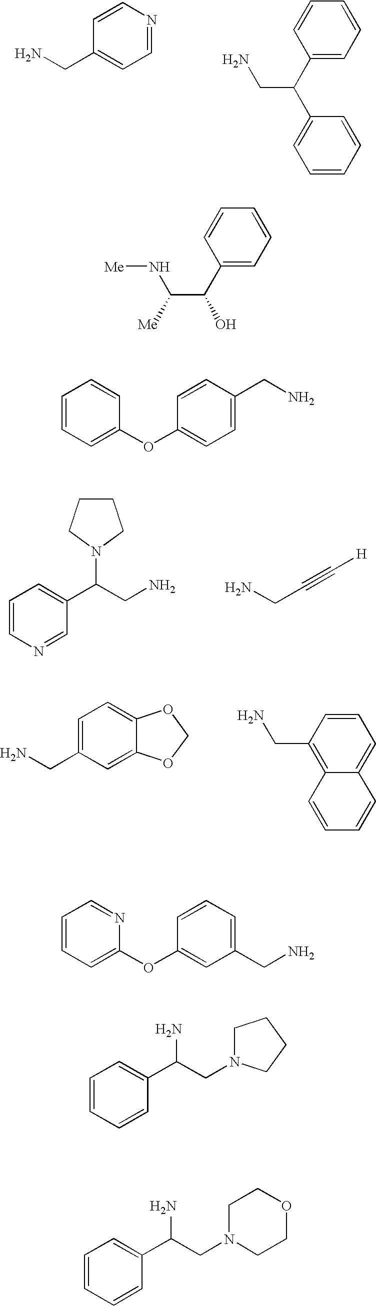

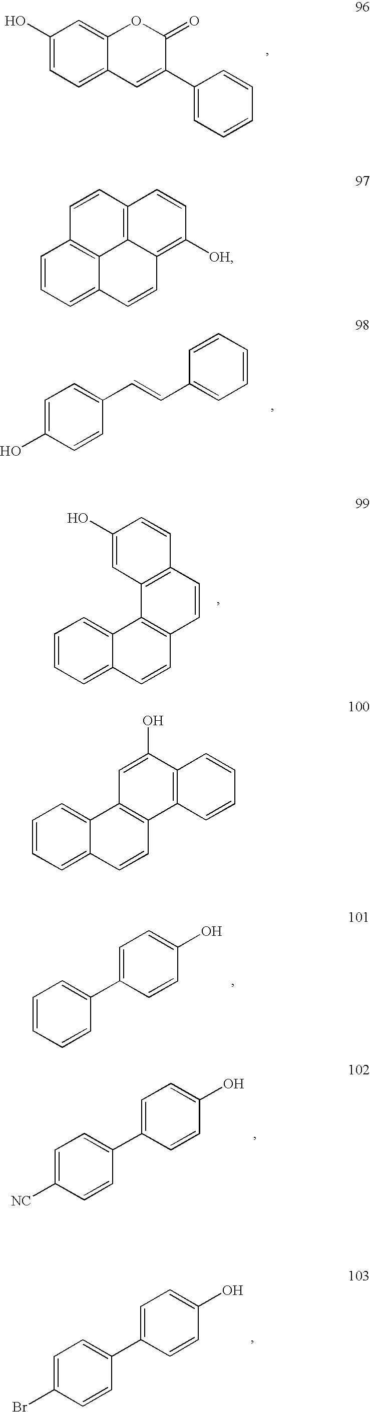

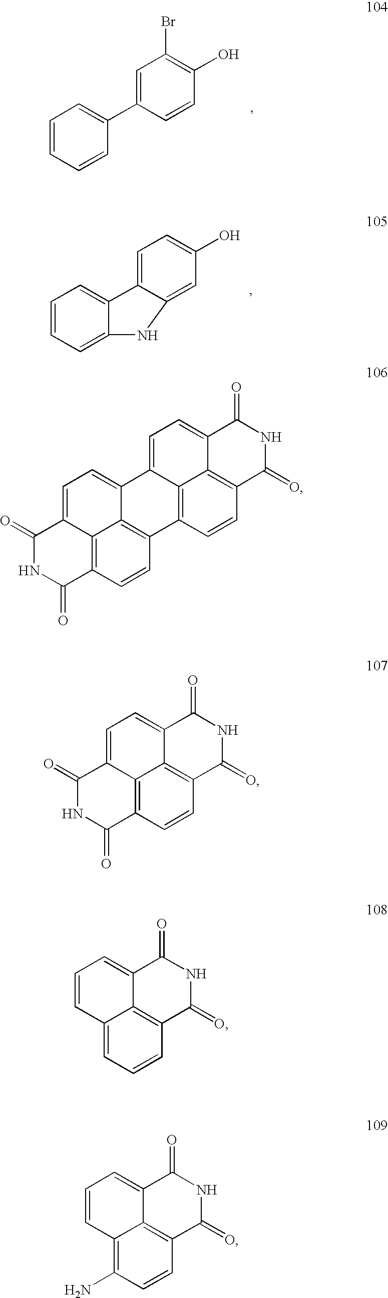

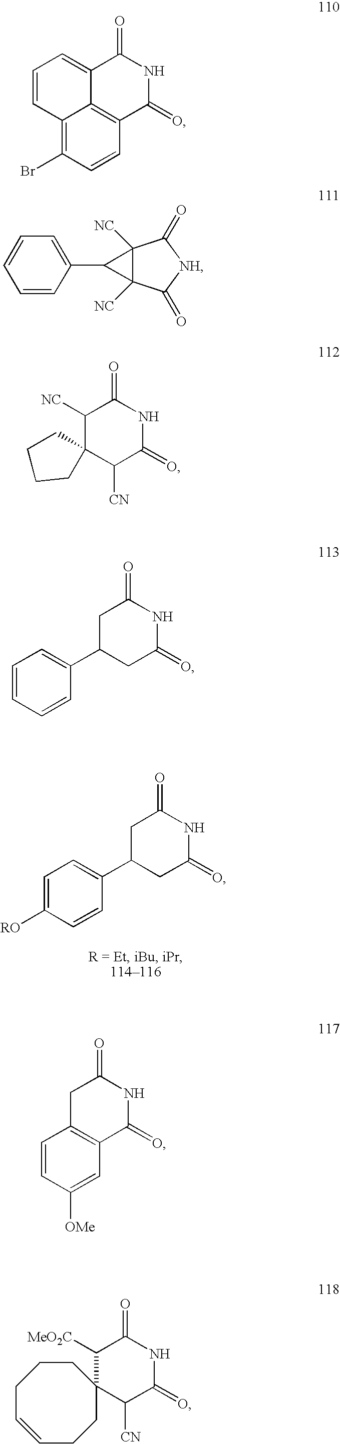

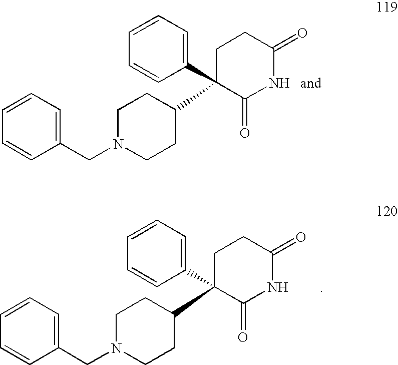

Particularly preferred compositions according the invention include a compound having the formula:

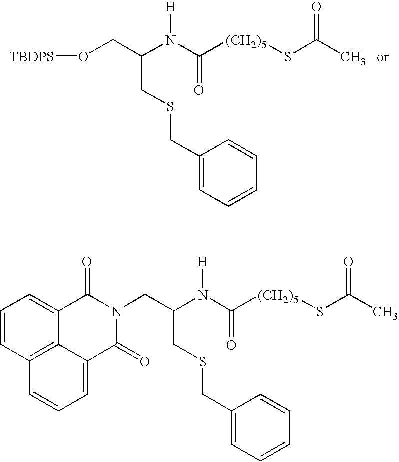

A particularly p referred composition according to the invention may have the formula:

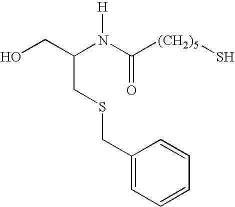

In another aspect, the present invention provides compositions for inhibiting a histone deacetylase comprising a cyclic FK228 analog represented by the general formula:

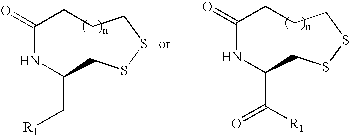

wherein n is an integer from 1 to 7; and

wherein R₁ is —OH, RCONH₂ or a cap structure wherein R is selected from the group consisting of unsubstituted and substituted alkyls, alkenyls, alkynyls, cycloalkyls, aryls, heterocyclyls and the cap structure is selected from the group consisting of unsubstituted and substituted alkyls, alkenyls, alkynyls, cycloalkyls, aryls, heterocyclyls, phthalimides, naphthalimides and polycyclic phenols.

A preferred n value for all compounds described and claimed herein is n=3. Certain cyclic disulfide compounds may further include R₁ as a cap structure selected from the group consisting of:

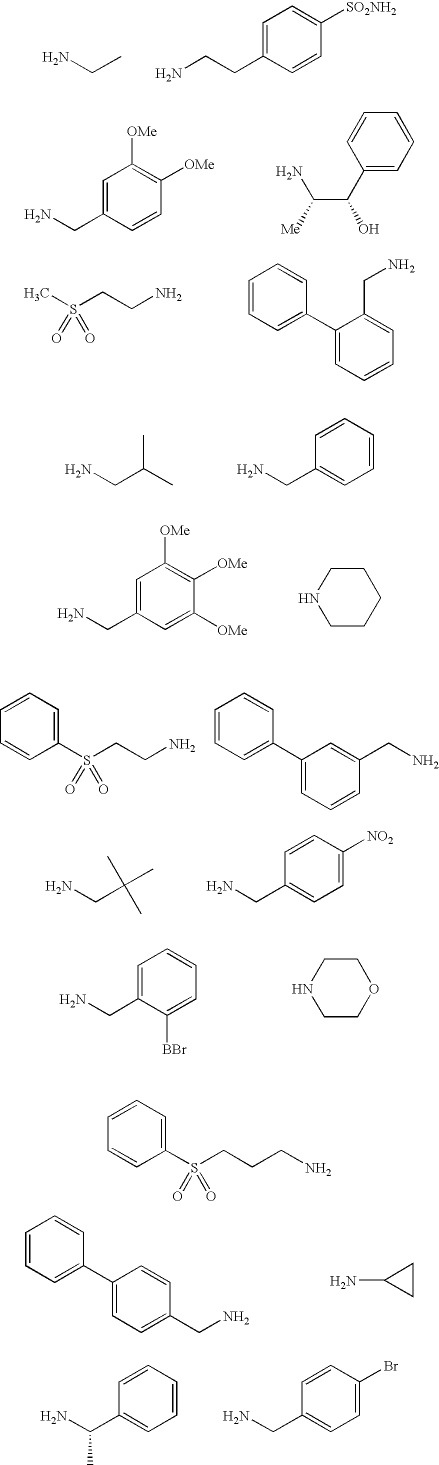

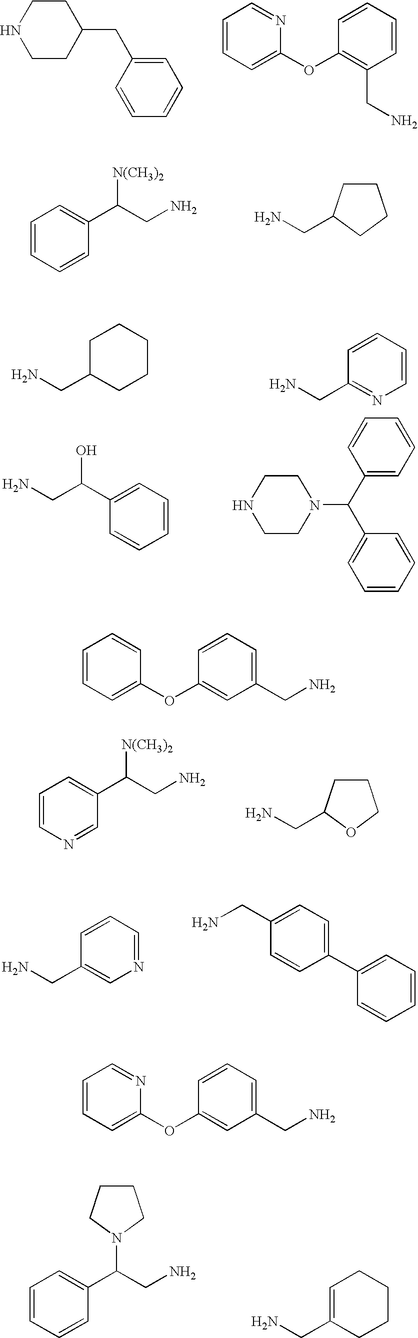

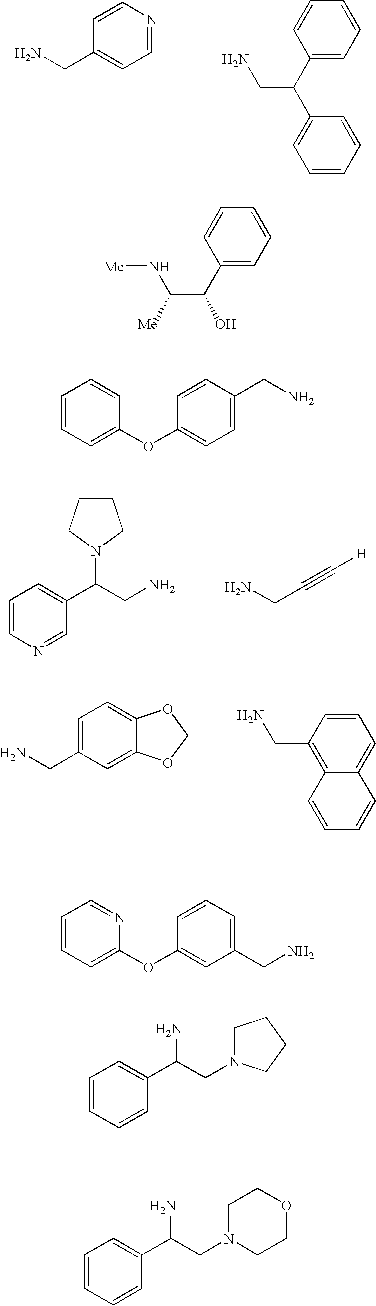

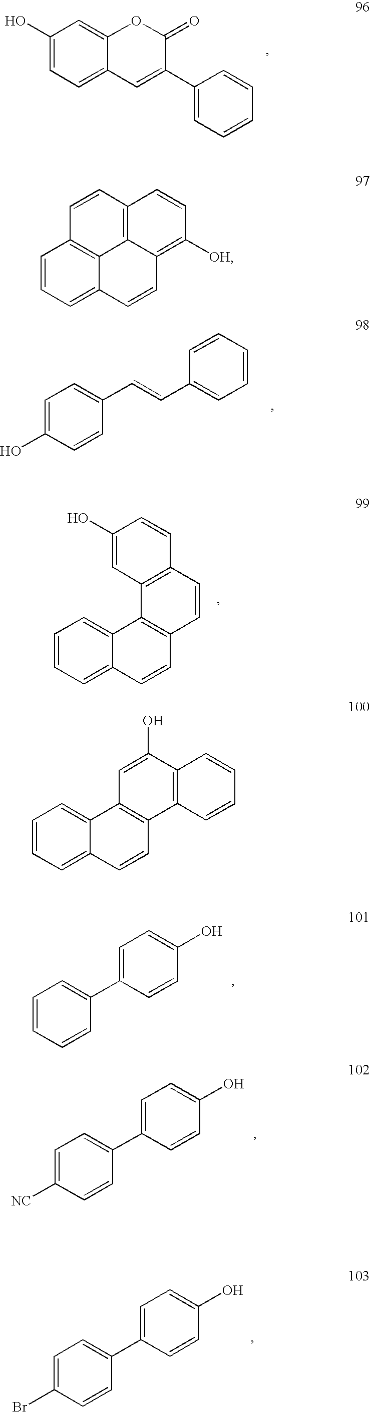

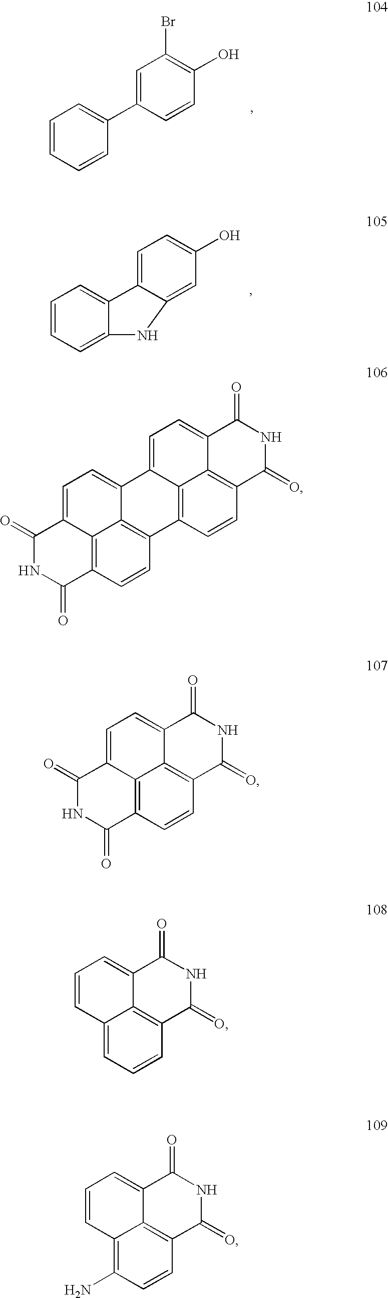

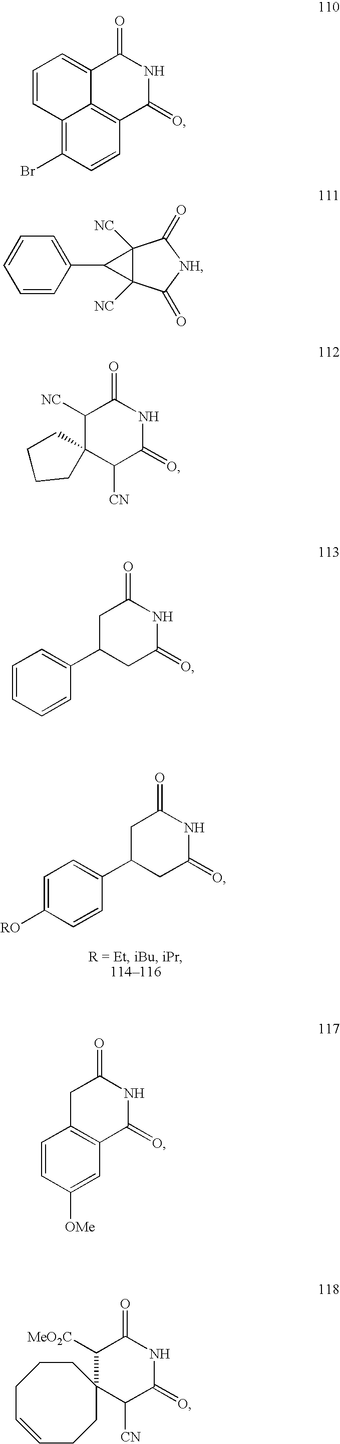

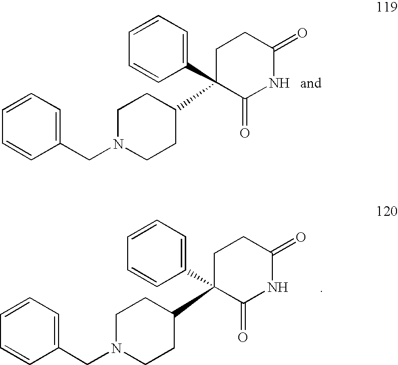

Also encompassed within the present invention are methods of synthesizing a cyclized disulfide compound for inhibiting a histone deacetylase. These methods include steps of chemically converting (a) a lactone to a corresponding TBS silyl ether lactone (b) the TBS silyl ether lactone to a corresponding acetylthiol; (c) the acetylthiol to a corresponding thiol; and (d) the thiol to a corresponding cyclized disulfide compound for inhibiting a histone deacetylase.

In a more preferred embodiment, the cyclized disulphide for inhibiting a histone deacetylase is synthesized by the following the method. The method includes the steps of chemically converting (a) a bromoacid to a corresponding ditritylated ester (b) the ditritylated ester to a corresponding macrocycle (c) the macrocycle to a corresponding cyclized disulphide compound via reduction for inhibiting a histone deacetylase.

Methods provided by the present invention for synthesizing cyclic disulfides may further include the additional step of coupling the cyclized disulfide compound to a targeting agent. Useful targeting agents include monoclonal antibodies or other agents known to accumulate in tumors, such as N-benzylpolyamines, porphyrins and related small molecules, as well as the capping structures disclosed herein.

Another embodiment of the present invention provides a compound produced by the method of any of claims 10 or 14, or paragraphs 19 and 20.

In yet another embodiment of the present invention, pharmaceutical compositions for inhibiting a histone deacetylase are provided which include compounds and compositions as described and claimed herein.

Another embodiment of the present embodiment includes a method of eliciting a chemopreventive effect for a disease in a patient comprising the step of administering a pharmaceutically effective amount of a composition according to the invention to a patient are further encompassed by the invention.

Other objects, features and advantages of the present invention will become apparent after review of the specification, claims and drawings.

### BRIEF DESCRIPTION OF THE DRAWINGS

FIG. 1 is a schematic representation of a mechanism depicting how, under normoxic conditions, HIF-1α is degraded by the ubiquitin-proteosome system in a process that relies upon the von Hippel Lindau (VHL) tumor suppressor protein.

FIG. 2 is a schematic representation depicting the use of ε-caprolactone to genrate free thiol 29. The inventors conducted the reaction sequence indicated in the scheme, and subsequently assayed for HDAC inhibition of simple C5 linked thiols, which are precursors to cyclic disulfides, to demonstrate that alkylthiols are effective HDAC inhibitors.

FIG. 3 depicts the bioactivity of disulfide precursors (A) 27, 28, and 33 and (B) 34, 31 and 32 using Drosophila S2 cells and immunoprecipitation studies.

FIG. 4 is a schematic representation depicting a general structure of a cyclic FK228 analog according to the invention.

FIG. 5 illustrates a partial library of cyclic disulfide FK228 analogs according to the invention.

FIG. 6(a) depicts the conversion of a lactone to a cyclic disulfide according to the present invention. (b) depicts a preferred method for construction of cyclic disulphides according to the present invention.

FIG. 7. Western blot analysis of histones isolated from Drosophila S2 cells treated with cyclic disulfides 125b-d

### DETAILED DESCRIPTION OF THE INVENTION

### I. IN GENERAL

Before the present compounds, compositions, methods and syntheses are described, it is understood that this invention is not limited to the particular methodology, protocols, and reagents described, as these may vary. One of ordiniary skill in the art may change mehtodology, synthetic protocols and reagents, as necessary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

It must be noted that as used herein and in the appended claims, the singular forms “a”, “an”, and “the” include plural reference unless the context clearly dictates otherwise. Thus, for example, reference to “a HDAC inhibitor” includes a plurality of such inhibitors and equivalents thereof known to those skilled in the art, and so forth. As well, the terms “a” (or “an”), “one or more” and “at least one” can be used interchangeably herein. It is also to be noted that the terms “comprising”, “including”, “characterized by” and “having” can be used interchangeably.

Although any methods and materials similar or equivalent to those described herein can be used in the practice or testing of the present invention, the preferred methods and materials are now described. All publications mentioned herein are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning: A Laboratory Manual, 2nd Ed., ed. by Sambrook, Fritsch and Maniatis (Cold Spring Harbor Laboratory Press, 1989); DNA Cloning, Volumes I and II (D. N. Glover ed., 1985); Oligonucleotide Synthesis (M. J. Gait ed., 1984); Mullis et al., U.S. Pat. No: 4,683,195; Nucleic Acid Hybridization (B. D. Hames & S. J. Higgins eds. 1984); Transcription and Translation (B. D. Hames & S. J. Higgins eds., 1984); Culture Of Animal Cells (R. I. Freshney, Alan R. Liss, Inc., 1987); Immobilized Cells And Enzymes (IRL Press, 1986); B. Perbal, A Practical Guide to Molecular Cloning (1984); the treatise, Methods In Enzymology (Academic Press, Inc., N.Y.); Gene Transfer Vectors For Mammalian Cells (J. H. Miller and M. P. Calos, eds., 1987, Cold Spring Harbor Laboratory); Methods In Enzymology, Vols. 154 and 155 (Wu et al., eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986).

In order to provide a clearer and consistent understanding of the specification and claims, including the scope to be given such terms, the following definitions are provided.

A “therapeutically effective amount” is the amount effective to inhibit the growth of the tumor(s) in vivo. An effective amount of a histone deacetylase inhibitor or an effective amount of a FK228 analog used as a histone deacetylase inhibitor is preferably the amount of either of these substances that is effective in inhibiting the growth of tumor(s) when administered to a patient suffering from a diseased state.

The term “alkyl” refers to the group of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 4-10 carbon atoms in their ring structure, and more preferable have 5, 6 or 7 carbons in the ring structure.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

Moreover, the term “alkyl” (or “lower alkyl”) as used throughout the specification and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, halogen, hydroxyl, carbonyl (such as a carboxylate, alkoxycarbonyl, aryloxycarbonyl, alkylcarbonyl, arylcarbonyl, aldehyde, and the like), thiocarbonyl (such as a thioacid, alkoxycarbonyl, and the like), an alkoxyl, unsubstituted amino, mono- or disubstituted amino, amido, amidine, imine, nitro, azido, sulfhydryl, alkylthio, cyano, trifluoromethyl, sulfonato, sulfamoyl, sulfonamido, heterocyclyl, aralkyl, or an aromatic or heteroaromatic moiety. It will be understood by those skilled in the art that the moieties substituted on the hydrocarbon chain can themselves by substituted, as described above, if appropriate. Exemplary substituted alkyls are described below. Cycloalkyls can be further substituted with, e.g., alkyls, alkenyls, alkoxys, alkylthios, aminoalkyls, carbonyl-substituted alkyls, —CF₃, —CN, and the like.

The terms “alkenyl” and “alkynyl” refer to unsaturated aliphatic groups analogous in length and possible substitution to the alkyls described above, but that contain at least one double or triple bond respectively. The term “enyne” refers to an unsaturated aliphatic moiety having at least one double bond and one triple bond.

The terms “alkylidene,” “alkenylidene,” and “alkynylidene” are art-recognized and refer to moieties corresponding to alkyl, alkenyl, and alkynyl moieties as defined above, but having two valences available for bonding.

The term “aryl” as used herein includes 5-, 6- and 7-membered ring aromatic groups that may include from zero to four heteroatoms, for example, phenyl, pyrrolyl, furanyl, thiophenyl, imidazolyl, oxazolyl, thiazolyl, triazolyl, pyraszolyl, pyridyl, pyrazinyl, pyrimidyl, and the like. Those aryl grups having heteroatoms in the ring structure may also be referred to as “aryl heterocycles” or “heteroaromatics”. The aromatic ring can be substituted at one or more ring positions with such substituents as described above, as for example, halogen, azido, alkyl, aralkyl, alkenyl, alkynyl, cycloalkyl, hydroxyl, amino, nitro, sulfhydryl, imino, amido, carboyl, carboxyl, silyl, ether, alkylthio, sulfonyl, sulfonamido, ketone, aldehyde, ester, a heterocyclyl, an aromatic or heteroaromatic moiety, —CF₃, —CN, or the like.

The term “aralkyl”, as used herein, refers to an alkyl group substituted with an aryl group (e.g., an aromatic or heteroaromatic group).

The terms “heterocyclyl” or “heterocyclic group” refer to non-aromatic 4- to 10-membered ring structures, more preferable 4- to 7-membered rings, which ring structures include one to four heteroatoms (e.g., O, N, S, P and the like). Heterocyclyl groups include, for example, pyrrolidine, oxolane, thiolane, imidazole, oxazone, piperidine, piperazine, morpholine, lactones, lactams such as azetidinones and pyrrolidinones, sultams, sultones, and the like. The heterocyclic ring can be substituted at one or more positions with such substitutents as described above, as for example, halogen, alkyl, aralkyl, alkenyl, alkynyl, cycloalkyl, hydroxyl, amino, nitro, sulfhydryl, imino, amido, alkoxycarbonyl, aryloxycarbonyl, carboxyl, silyl, ether, alkylthio, alkylsulfonyl, arylsulfonyl, ketone (e.g., —C(O)-alkyl or —C(O)-aryl), aldehyde, heterocyclyl, an aryl or heteroaryl moiety, —CF₃, —CN, or the like.

The term “novel” FK228 analogs (or compounds), as used herein, refer to FK228 analogs which have not been made in vitro prior to the teachings of the present inventors in the references cited herein, or to FK228 analogs which have never been produced synthetically prior to the teachings of the present inventors in the references cited herein, or to FK228 analogs that are completely novel and have never been produced via natural or chemical syntheses.

### II. HISTONE DEACETYLASES

Angiogenesis, hypoxia and HIF-1 are coupled through the actions of histone deacetylases 1 (HDAC1). The growth of new blood vessels into a cancer (angiogenesis) is required for continued growth of the tumor mass beyond 1-2 mm³. Increased numbers of blood vessels in breast cancer, and other cancers as well, correlates closely with metastasis and poor prognosis. Tumor hypoxia is a major inducer of vascular endothelial growth factor (VEGF) gene expression (Kim et al., 2001, Nature Medicine 7: 437-443). VEGF expression is under the control of the hypoxia-inducible factor (HIF-1), a heterodimeric transcription factor recognized as the key regulator of the hypoxia response in a variety of cell types (Kim et al., 2001, Nat. Med. 7: 437-443; Semenza, 2000, Cancer and Metastatis Rev. 19: 56-65; Semenza, 2001, Curr. Op. Cell Biol. 13: 167-171; Ratcliffe et al., 2000, Nat. Med. 6: 315-1316). Composed of HIF-1α and HIF-1β, HIF-1 activates the transcription of genes encoding angiogenic growth factors and vasomotor regulators. HIF-1 also regulates the expression of molecules involved in matrix modeling, iron transport/regulation and apoptosis/cell) proliferation (Semenza, 2000, Cancer and Metastatis Rev. 19: 56-65; Semenza, 2001, Curr. Op. Cell Biol. 13: 167-171; Ratcliffe et al., 2000, Nat. Med. 6: 1315-1316). HIF-1α is constitutively expressed, whereas HIF-1β is induced by exposure of cells to hypoxia or growth factors. Importantly, HIF expression levels are characteristically increased in many cancerous tumor types as are a number of reductases (Saramaki et al., 2001, Cancer Gen. and Cytogen. 128: 31-34; Huss et al., 2001, Cancer Res. 61: 2736-2743; Cvetkovic et al., 2001, Urology 57: 821-825).

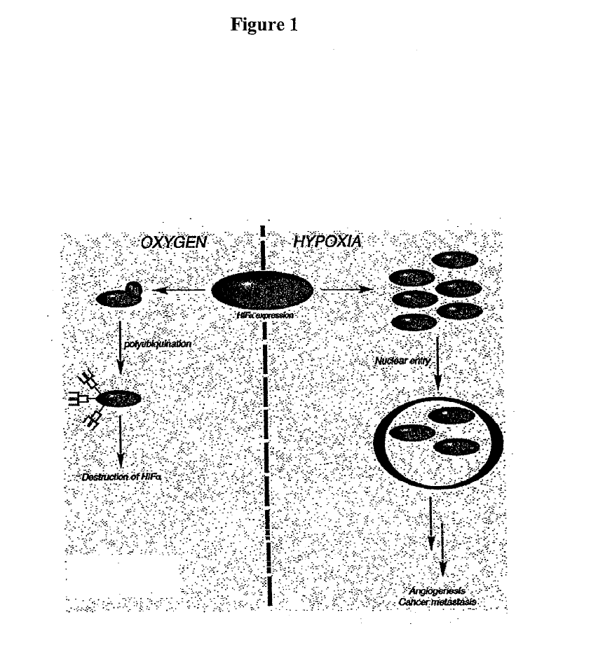

In the mechanism depicted in FIG. 1, under normoxic conditions, HIF-1α is degraded by the ubiquitin-proteosome system. This process relies upon the von Hippel Lindau (VHL) tumor suppressor protein; interaction with HIF-1α affords the recognition component of an E3 ubiquitin ligase complex (Kim et al., 2001, Nat. Med. 7: 437-443). Hypoxia-associated reduction of VHL levels leads to HIF-1α accumulation and subsequent overexpression of proangiogenic (metastasis-associated) agents. Hypoxia and HIF-1α overexpression are hallmarks of many tumor types, particularly prostate carcinomas (Saramaki et al., 2001, Cancer Gen. and Cytogen. 128: 31-34; Cvetkovic et al., 2001, Urology 57: 821-825).

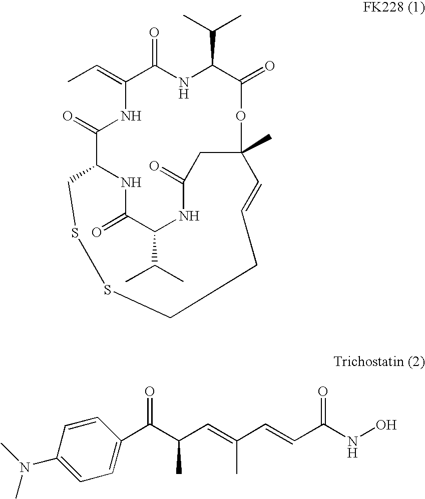

As noted above, hypoxia, HIF-1α and angiogenesis are all coupled by the actions of histone deacetylase 1 (HDAC1; Kim et al., 2001, Nat. Med. 7: 437-443; Williams, 2001, Expert Opin. Invest. Drugs 10: 1571-1573; Furumai et al., 2002, Cancer Res. 62: 4916-4921). Hypoxia-dependent upregulation of HDAC1 negatively regulates VHL levels which in turn enhances HIF-1α. It is highly significant that this was realized only after extensive mechanism of action studies on depsipeptide FK228 (1) and trichostatin A (TSA) (2) (Furumai et al., 2002, Cancer Res. 62: 4916-4921; Kwon et al., 2002, Int. J. Cancer 97: 290-296). Both natural product inhibitors of HDAC1 display potent antiangiogenic properties at well below toxic levels (Kim et al., 2001, Nat. Med. 7: 437-443; Saramaki et al., 2001, Cancer Gen. and Cytogen. 128: 31-34; Furumai et al., 2002, Cancer Res. 62: 4916-4921; Kwon et al., 2002, Int. J. Cancer 97: 290-296). FK228-treated HeLa cells reveal significant reductions in the angiogenic-stimulating factors VEGF, FLK-1 and VEGF receptor (Kwon et al., 2002, Int. J. Cancer 97: 290-296). Conversely, antiangiogenic factors such as VHL and neurofibromin2 (NF2) are upregulated in comparison to cells devoid of FK228 treatment. Examination of TSA's antiangiogenic activity revealed similar reductions in VEGF, the result of TSA-dependent “re-expression” of VHL and p53 (Kim et al., 2001, Nat. Med. 7: 437-443). These small molecule studies have led to a clearer understanding of the relationship between angiogenic promoters, hypoxia and HDAC1. More importantly, they illustrate the role that cell-permeable small molecule inhibitors of HDACs can play as a new class of antiangiogenic agents. This is particularly important due to the heterogeneity of endothelial cells (ECs) (Klohs and Hamby, 1999, Curr. Op. Biotech. 10: 544-549). This heterogeneity represents a major obstacle to antiangiogenic therapies. No single antiangiogenic agent alone will be effective against all cancers. Drugs that exploit different mechanistic paths need to be developed in order to better address and circumvent issues of EC and tumor heterogeneity. Ideally, each drug class would be easily synthesized and readily diversified to meet the changing needs of specific tumor targets.

TSA and FK228 do not exclusively target HDAC1. That HDAC1 inhibition by TSA is not specific to hypoxic cells is consistent with the notion that 2, like most effective HDAC inhibitors (IC₅₀≦50 nM), does not require an activating event en route to expression of bioactivity (Breslow et al., 2000, Helv. Chim. Acta 83: 1685-1692; Marks et al., 2001, Curr. Op. Oncol. 13: 477-483; Hassig and Schreiber, 1997, Curr. Op. Chem. Biol. 1, 300-308-; Hung et al., 1996, Chem. Biol. 3: 623-639; Pazin et al., 1997, Cell 89: 325-328). FK228 (1) is the one exception to this rule; intracellular disulfide→dithiol conversion is required for effective HDAC inhibition and is at the heart of FK228s very promising future (Furumai et al., 2002). FK228 does in fact require intracellular disulfide cleavage, but this provides the lone example of a bioactivated HDAC inhibitor.

Trx is found in high levels in many human cancers and increases both aerobic and hypoxia-induced HIF-1α concentrations promoting the notion that Trx-activated agents are likely to display beneficial tumor cell selectivity (Shao et al., 2001; Becker et al., 2000; Arner et al., 2000). The likelihood that FK228 undergoes Trx-mediated activation has been proposed as a critical element behind its potent antitumor activity (Furumai et al., 2002; Kwon et al., 2002).GSH has also been implicated in FK228 activation and presents another possible manifold by which tumor cell selectivity arises. This is particularly significant since enhanced GSH levels represent one manifold by which drug resistance arises in tumor cells.

An intracellular redox change is therefore at the heart of FK228's very promising future (Furumai et al., 2002, Cancer Res. 62: 4916-4921). FK228 is currently in Phase I clinical trials for thyroid and other advanced malignancies, combination therapy for lung cancer, and also for leukemias. The agent is in Phase II clinical trials for T cell lymphomas and other phase II projections involving Non-Hodgkins lymphoma and acute myelogenous leukemia, and pancreatic cancer. Alarmingly, many of these trials have been hindered due to a shortage of the natural product (Li et al., 1996, J. Am. Chem. Soc. 118: 7237-7238).

HDACs mediate gene expression through deacetylation of N-acetyl lysine residues contained within histone proteins and other transcriptional regulators (Prives and Manley, 2001, Cell 107: 815-818). How this covalent modification to histone proteins elicits changes at the transcriptional level is not mechanistically well understood; a lack of HDAC-specific inhibitors is largely to blame.

Highly refined tools for the global analysis of HDAC function are now available yet tools with which to perturb HDAC function are still in their infancy. Methods for the temporal control of gene expression would allow the differentiation between direct, early effects and indirect, late effects and are most certainly needed to formulate coherent drug design and discovery processes that capitalize on HDACs. Deregulation of HDAC activity/function is implicated in a wide array of malignant diseases (Kwon et al., 2002, Int. J. Cancer 97: 290-296; Dieter, 2000, Mol. Med. 6: 623-644; Kelly et al., 2002, Expert Op. Invest. Drugs 11: 1695-1713; Vigushin et al., 2002, 13: 1-13; Minucci et al., 2001, Oncogene 20: 3110-3115). Recently, HDACs have also been found to be overexpressed under specific environmental conditions such as hypoxia, hypoglycemia and serum deprivation and it is now also apparent that HDAC inhibitors may have use as agents to combat infectious disease (Smith et al., 2002, Antimicrobial Agents & Chemotherapy 46: 3532-3539; Klar et al., 2001, Genetics 158: 919-924; Andrews et al., 2000, Int. J. Parasit. 30: 761-768). Agents with discreet specificity both at the enzyme and/or cellular levels would be extremely valuable both as tools for probing the biological functions of HDACs and also for therapeutic purposes such as inhibiting potentially disease-specific HDACs (Grozinger and Schreiber, 2002, Chem. Biol. 9: 3-16; Tong, 2002, Chem. Biol. 9: 668-670).

These findings suggest that inhibition of HDAC activity represents a novel approach for intervening in cell cycle regulation and that HDAC inhibitors have great therapeutic potential in the treatment of cell proliferative diseases or conditions.

### III. HISTONE DEACETYLASE INHIBITORS

A tripartate structure characterizes the majority of effective HDAC inhibitors (Breslow et al., 2000, Helv. Chim. Acta 83: 1685-1692; Marks et al., 2001, Curr. Op. Oncol. 13: 477-483). An HDAC recognition or affinity “cap” is attached to an enzyme active site binding/inactivating group (3, boxed region), via a linker devoid of elaborate functionality (Breslow et al., 2000, Helv. Chim. Acta 83: 1685-1692; Marks et al., 2001, Curr. Op. Oncol. 13: 477-483; Taunton et al., 1996b, J. Am. Chem. Soc. 118: 10412-22;). The length and linearity of the linker are crucial for efficient HDAC inhibition. This is explained, in part, by a necessary resemblance to ε-N-acetyl lysine, the substrate for native HDAC action (Breslow et al., 2000, Helv. Chim. Acta 83: 1685-1692; Marks et al., 2001, Curr. Op. Oncol. 13: 477-483; Taunton et al., 1996, J. Am. Chem. Soc. 118: 10412-22;). Crystallographic studies of TSA, (2) and the clinical candidate SAHA (5), bound to an HDAC homologue, support this amino acid mimicry (Finnin et al., 1999, Nature 401: 188-913). The hydroxamate pharmacophore of each agent binds an active site zinc ion thus abrogating HDAC function. Deviation from linearity shortens the distance between the recognition cap and active site binding groups thus rendering biologically inactive molecules (Breslow et al., 2000, Helv. Chim. Acta 83: 1685-1692). Studies of hybrid HDAC inhibitors support this notion. Replacement of the epoxyketone of trapoxin (3) with the hydroxamate pharmacophore of TSA affords HDAC inhibitors with extraordinary IC₅₀ values (Furumai et al., 2001, Proc. Natl. Acad. Sci. USA 98: 87-92). This is in agreement with competition assays indicating that TSA and trapoxin B share the same site of binding/inhibition for HDAC1 (Taunton et al., 1996, J. Am. Chem. Soc. 118: 10412-22; Kwon et al., 1998, Proc. Natl. Acad. Sci. USA 95: 3356-3361).

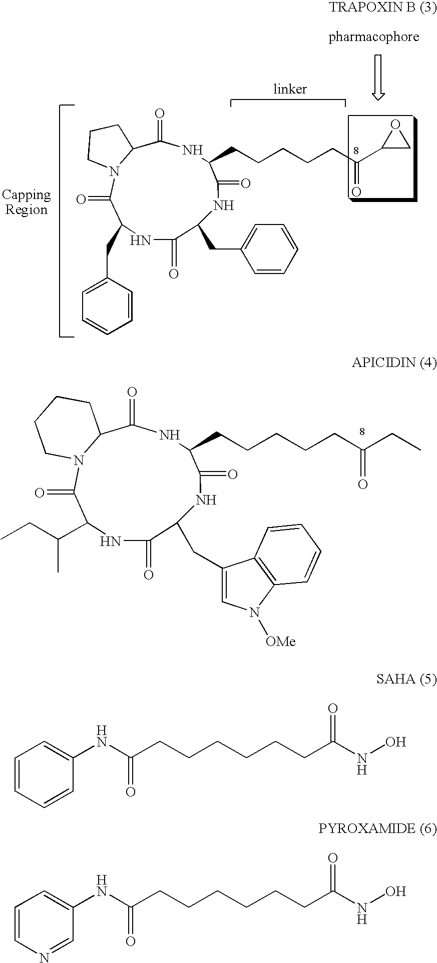

Structure/activity studies with hybrid HDAC inhibitors reveal a marked importance to linker length en route to effective HDAC inhibition. Just a one carbon difference in linker length renders an order of magnitude difference in HDAC1 IC₅₀ value (Kwon et al., 1998, Proc. Natl. Acad. Sci. USA 95: 3356-3361). Although workers in the field have identified the importance of linker length and linearity, synthetic efforts to derive HDAC/cell specificity have focused almost exclusively upon functionality composing either the “cap” region or the active site binding pharmacophore. For instance, N-alkylated indole analogs of apicidin show a greater than 20-fold preference for targeting malarial HDACs over human HDAC1 (Colletti et al., 2000, Tet. Lett. 41: 7825-7829). Efforts to make and identify HDAC inhibitors with useful specificity or selectivity continue to focus on new types of structures yet the lesson taught by FK228 that linker restriction via cyclization can lead to triggerable inhibitors appears to have gone either unnoticed or unheeded. It is also not established if, by virtue of their obligate tripartate structure, therapeutically useful HDAC inhibitors might be triggered by redox enzymes that are overexpressed by tumor cells (Husbeck and Powis, 2002, Carcinogenesis 23: 1625-1630; Kress, 1997, Cancer Res. 57: 1264-1269).

### IV. EMBODIMENTS OF THE INVENTION

The present invention provides compounds that are analogs of FK228, and methods for their synthesis as well as methods for their use.

In one aspect, the present invention provides compositions for inhibiting a histone deacetylase based upon novel structurally simple analogs of FK228. These compositions comprise a novel analog represented by the general formula:

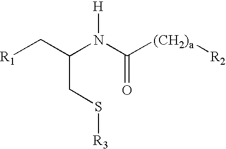

wherein R₁ is —OH, NH, NHR,

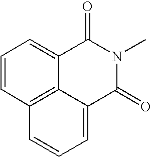

or a cap structure selected from the group consisting of unsubstituted and substituted alkyls, alkenyls, alkynyls, cycloalkyls, aryls, heterocyclyls, phthalimides, naphthalimides and polycyclic phenols;

wherein R₂ is a —SH or —SCOCH₃;

wherein R₃ is —H or a structure selected from the group consisting of unsubstituted and substituted alkyls, alkenyls, alkynyls, cycloalkyls, aryls, and heterocyclyls; and

wherein n is an integer from 1 to 7.

In a preferred composition according to the invention, the compound includes an R₁ group that is a tert-butyidiphenylsilyl (TBSPS—O—) group or

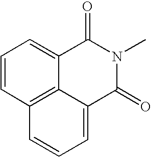

In other preferred compositions according to the invention, R₁ is a cap structure selected from the group consisting of:

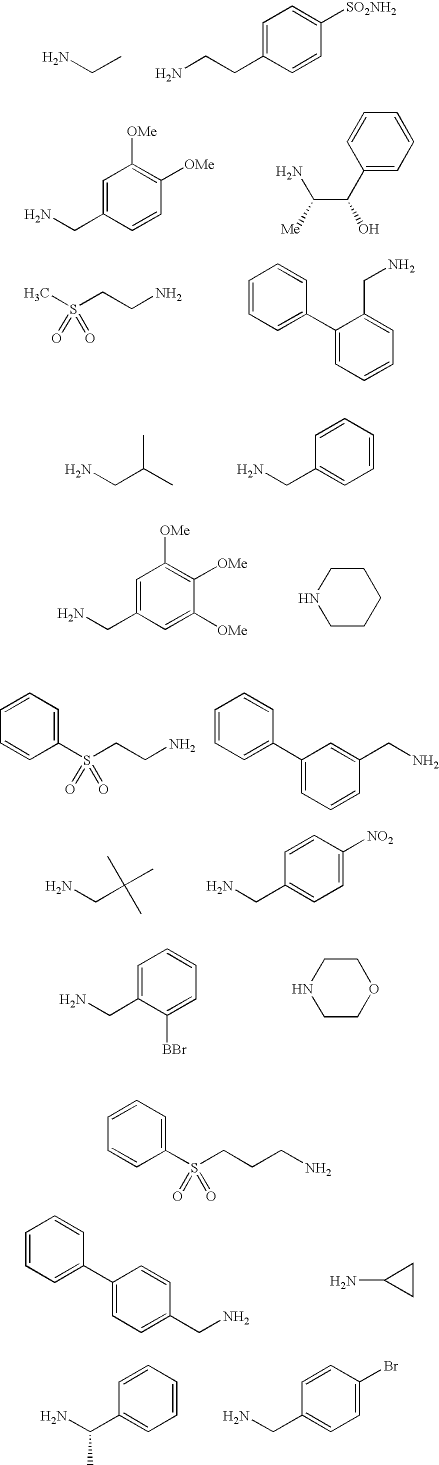

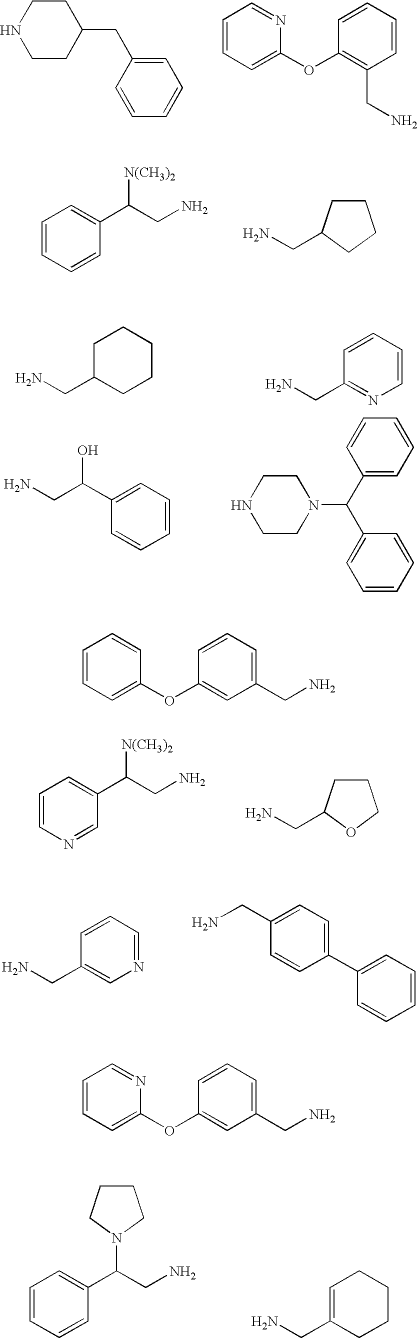

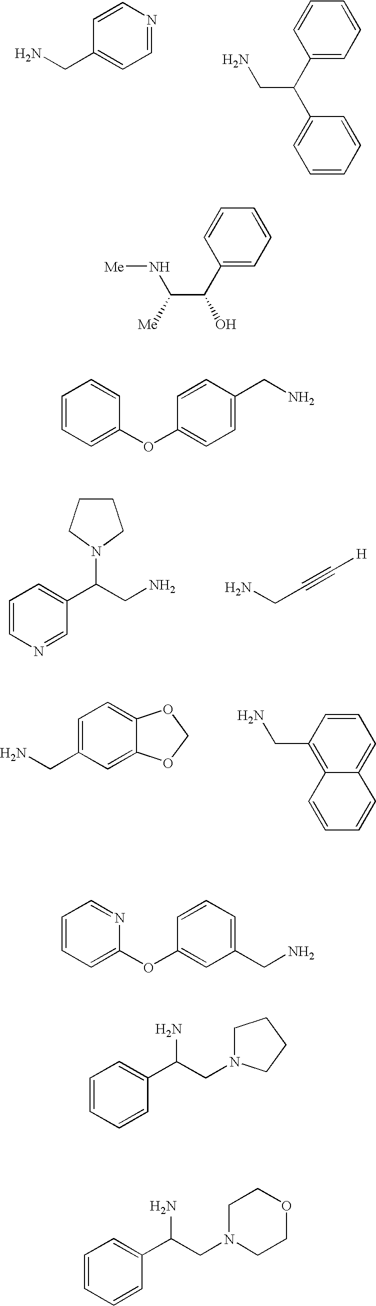

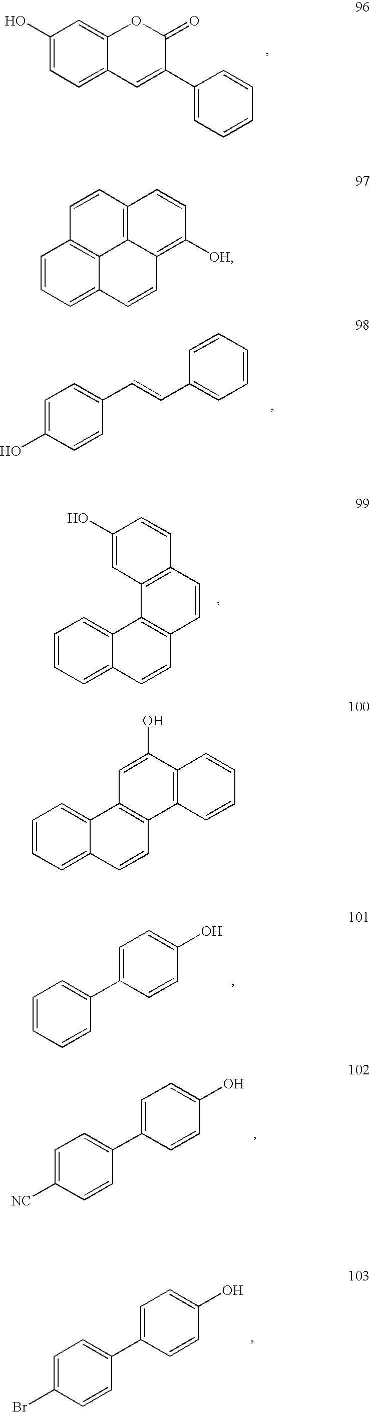

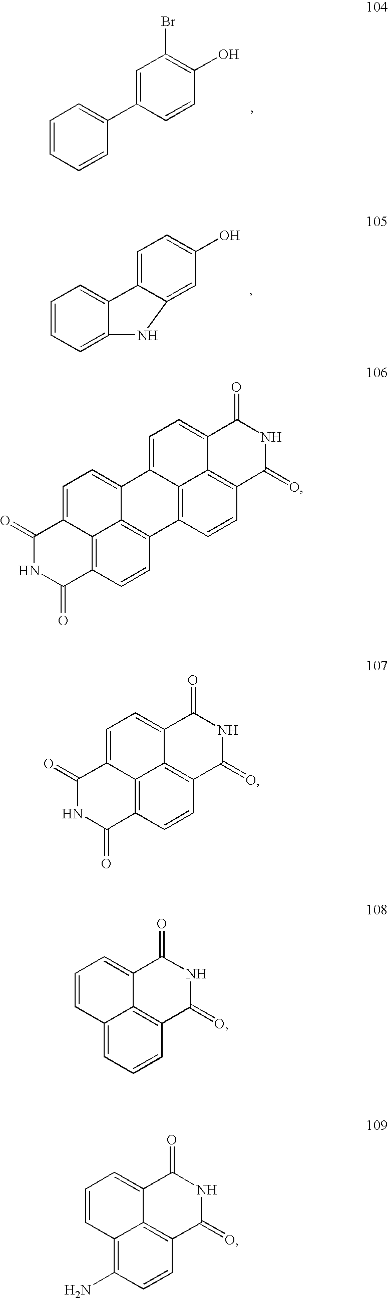

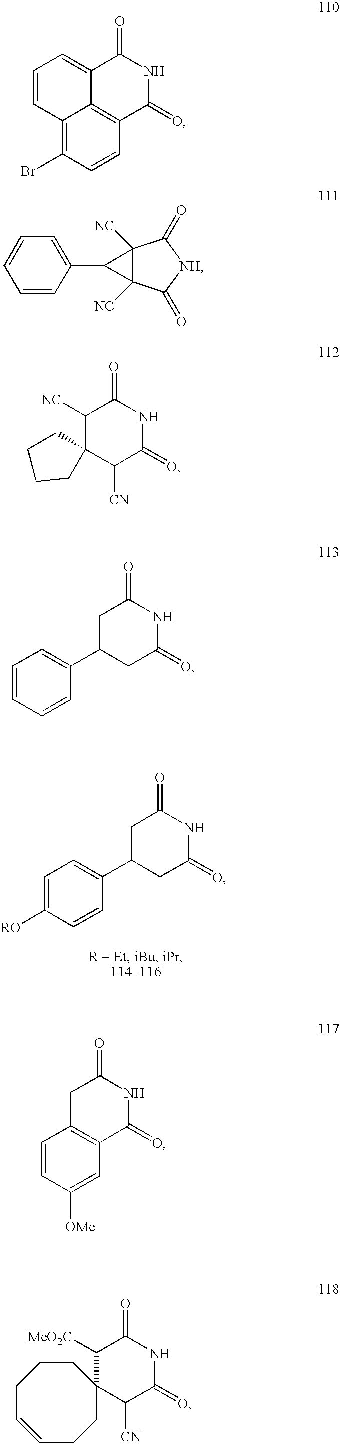

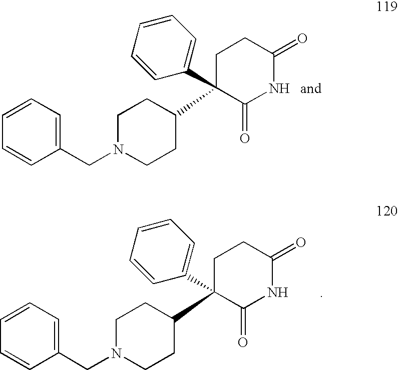

Particularly preferred compositions according the invention include a compoud having the formula:

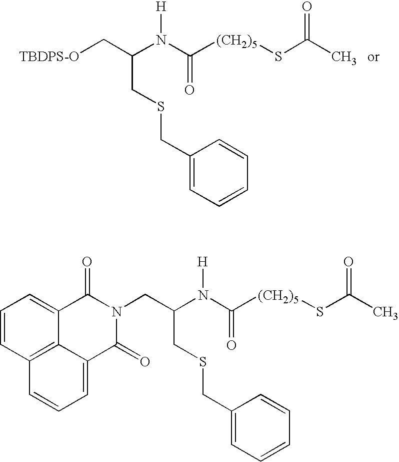

A particularly preferred composition according to the invention may have the formula:

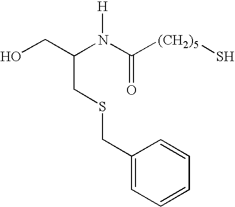

In another aspect, the present invention provides compositions for inhibiting a histone deacetylase comprising a cyclic FK228 analog represented by the general formula:

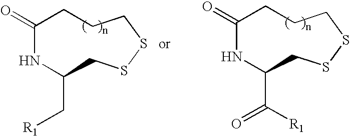

wherein n is an integer from 1 to 7; and

wherein R₁ is —OH, RCONH₂ or a cap structure wherein R is selected from the group consisting of unsubstituted and substituted alkyls, alkenyls, alkynyls, cycloalkyls, aryls, heterocyclyls and the cap structure is selected from the group consisting of unsubstituted and substituted alkyls, alkenyls, alkynyls, cycloalkyls, aryls, heterocyclyls, phthalimides, naphthalimides and polycyclic phenols.

A preferred n value for all compounds described and claimed herein is n=3. Certain cyclic disulfide compounds may further include R₁ as a cap structure selected from the group consisting of:

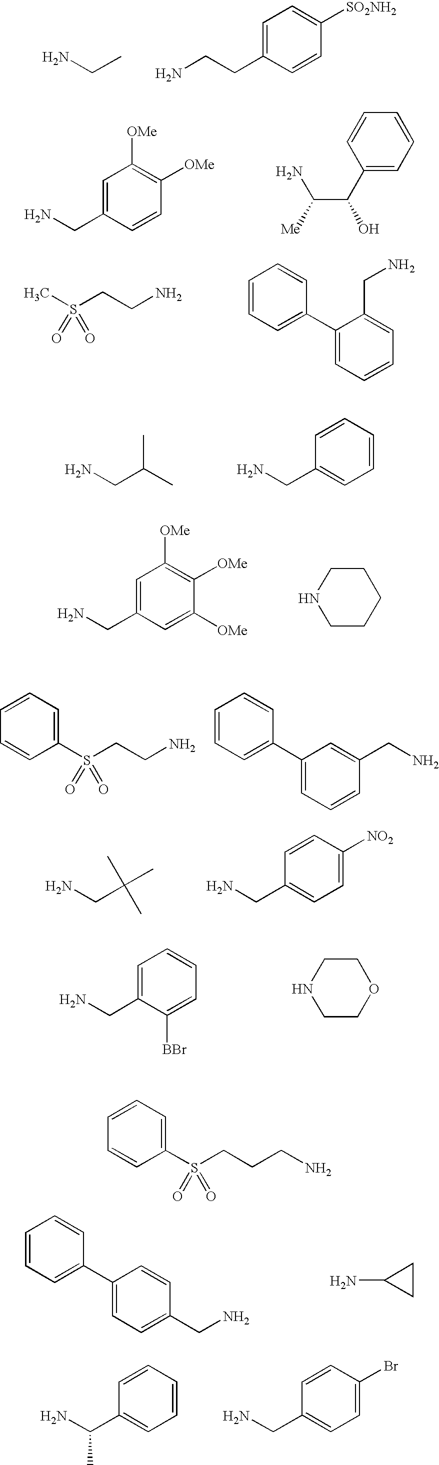

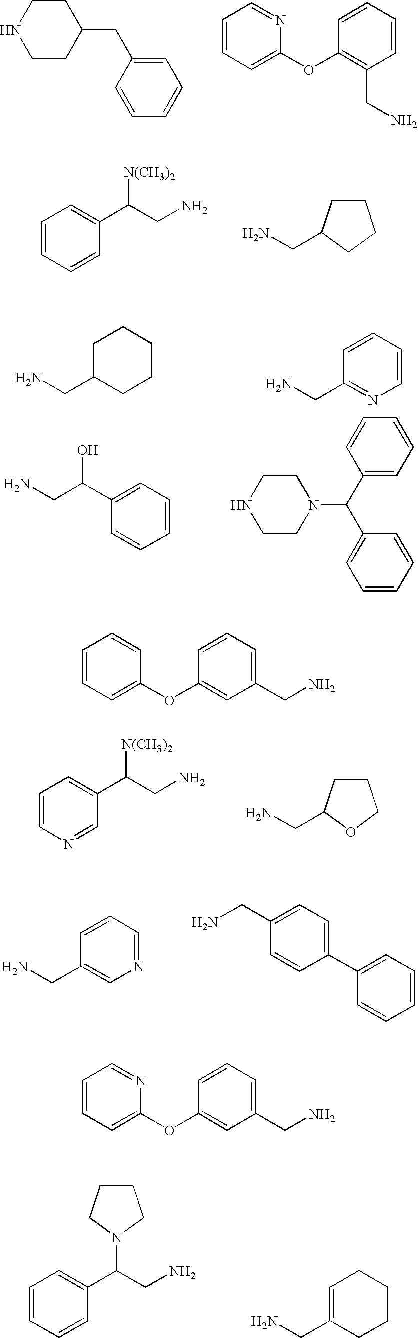

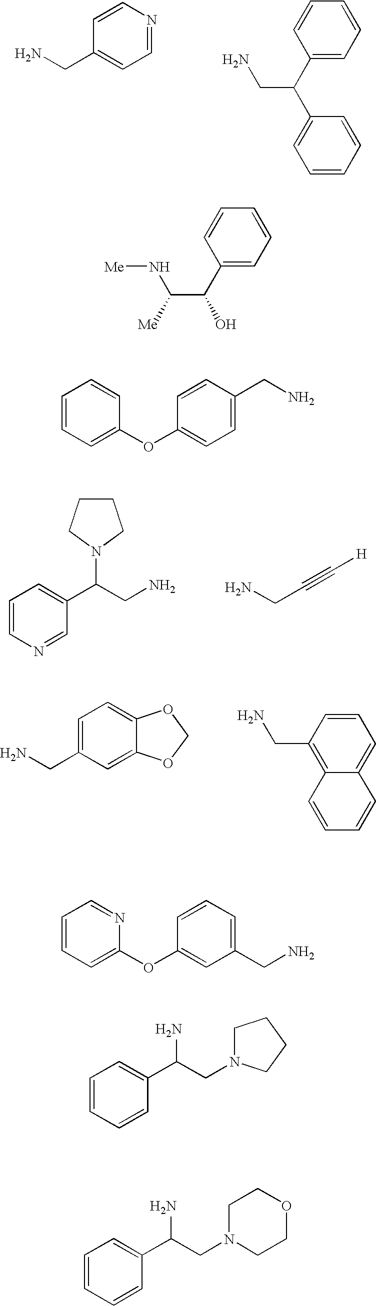

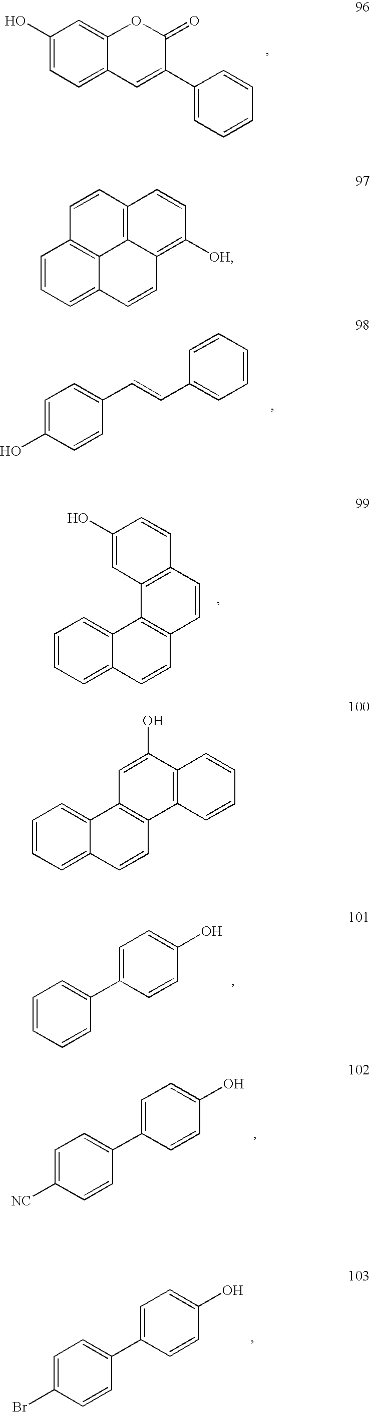

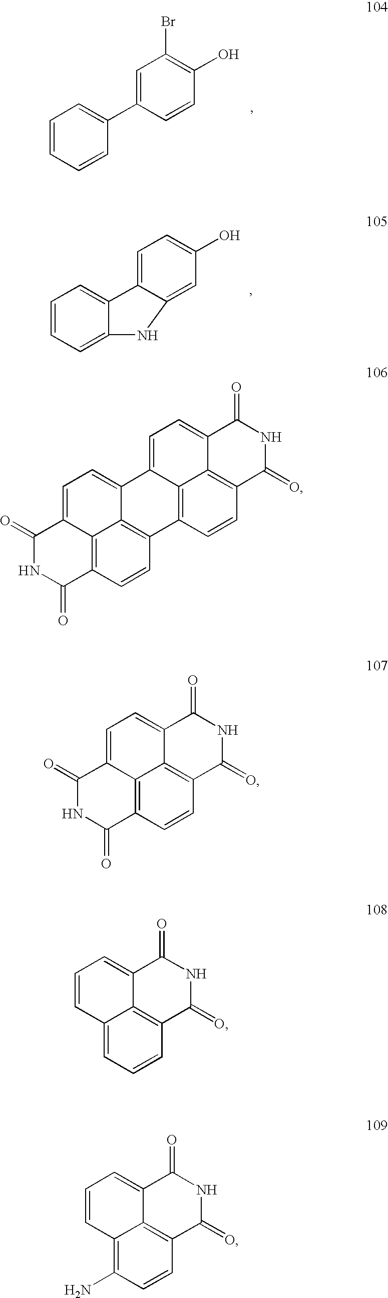

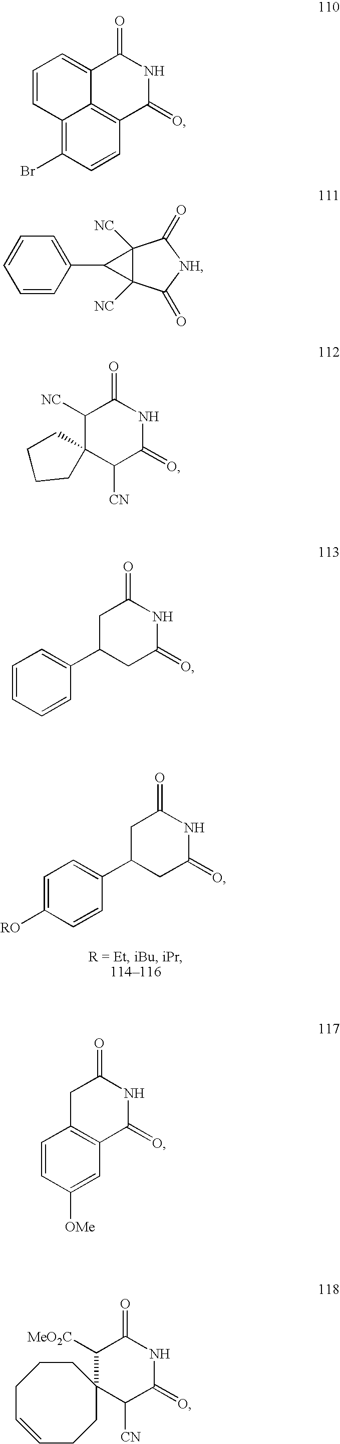

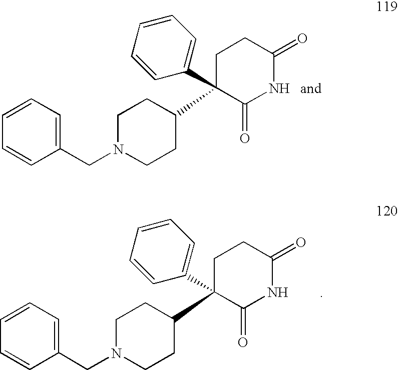

Also encompassed within the present invention are methods of synthesizing a cyclized disulfide compound for inhibiting a histone deacetylase. These methods include steps of chemically converting (a) a lactone to a corresponding TBS silyl ether lactone (b) the TBS silyl ether lactone to a corresponding acetylthiol;

(c) the acetylthiol to a corresponding thiol; and (d) the thiol to a corresponding cyclized disulfide compound for inhibiting a histone deacetylase.

In a more preferred embodiment, the cyclized disulphide for inhibiting a histone deacetylase is synthesized by the following the method. The method includes the steps of chemically converting

(a) a bromoacid to a corresponding ditritylated ester (b) the ditritylated ester to a corresponding macrocycle (c) the macrocycle to a corresponding cyclized disulphide compound via reduction for inhibiting a histone deacetylase.

Methods provided by the present invention for synthesizing cyclic disulfides may further include the additional step of coupling the cyclized disulfide compound to a targeting agent. Useful targeting agents include monoclonal antibodies or other agents known to accumulate in tumors, such as N-benzylpolyamines, porphyrins and related small molecules, as well as the capping structures disclosed herein.

Further, the cyclized disulphide compound may also have a capping group as discussed above.

Another embodiment of the present invention provides a compound produced by the method of any of claims 10 or 14, or paragraphs 19 and 20.

In yet another embodiment of the present invention, pharmaceutical compositions for inhibiting a histone deacetylase are provided which include compounds and compositions as described and claimed herein.

Another embodiment of the present embodiment includes a method of eliciting a chemopreventive effect for a disease in a patient comprising the step of administering a pharmaceutically effective amount of a composition according to the invention to a patient are further encompassed by the invention.

The invention is preferably directed to synthesis and evaluation of redox activated histone deacetylase (HDAC) inhibitors. As discussed above, trapoxin B (3) and depsipeptide FK228 (1) display impressive anticancer activities by virtue of their capacity for HDAC inhibition. However, of the HDAC inhibitors known, only FK228 appears to have any cell selectivity. Intracellular cleavage of the FK228 disulfide allows for linker linearization and subsequent active site metal binding by the thiol capped butene moiety. Based upon the innovative concept of “reductive linker linearization,” the inventors are developing glutathione (GSH)-activated HDAC inhibitors, which are antiangiogenic and antimetastatic agents based upon structural variants of 1. These inhibitors capitalize on the overexpression of abundant GSH levels that typify many tumor cell types. In addition, identification of HDAC-selective agents provide useful biochemical tools.

The present invention further includes pharmaceutical compositions for the treatment or prophylaxis of tumor, inflammatory disorders, diabetes, diabetic complication, homozygous thalassemia, fibrosis, cirrhosis, acute promyelocytic leukemia, organ transplant rejection, and autoimmune disease. Pharmaceutical composition, according to the invention, comprise an FK228 analog or salts thereof as an active ingredient.

In a preferred embodiment, this invention teaches HDAC-inhibiting cyclized prodrugs having functional analogy to FK228. Linker restriction of structurally diverse HDAC inhibitors is used to obtain cell selectivity on the basis of altered redox enzyme expression levels. The invention encompasses the generation of HDAC inhibitors capable of expressing activity only after an S—S bond scission events. Conformational restriction of the linker portion of HDAC inhibitors analogous to FK228 will inhibit the expression of significant biological activity at undesired cellular locations. The inability of the linkage between drug pharmacophore and cap unit to assume a linear conformation will prohibit delivery of the pharmacophore to the HDAC active site. The enzymatic action of redox active enzymes overexpressed by hypoxic tumor cells will relieve this conformational restriction via cleavage of the labile bond (Saramaki et al., 2001, Cancer Gen. and Cytogen. 128: 31-34; Huss et al., 2001, Cancer Res. 61: 2736-2743; Cvetkovic et al., 2001, Urology 57: 821-825; Husbeck and Powis, 2002, Carcinogenesis 23: 1625-1630; Elaut et al., 2002, Metabolism and Disposition 30: 1320-1328), through either reductive or oxidative conditions. Linearization of the once constrained linker thusly affords a novel species capable of potent HDAC inhibition. The cellular consequence of this chemistry is inhibition of tumor-promoted angiogenesis via VHL-dependent destruction of HIF-1α (Ratcliffe et al., 2000, Nat. Med. 6: 1315-1316; Klohs and Hamby, 1999, Curr. Op. Biotech. 10: 544-549).

While not adopting any one theory of operation, the disulfide-containing reductase thioredoxin is alleged to play an important role in the favorable attributes of FK228 (Furumai et al., 2002, Cancer Res. 62: 4916-4921). Trx overexpression is associated with enhanced glutathione levels by virtue of glutathione reductase “over-activation”. The nucleophilic mechanism by which Trx reduces cellular proteins supports the notion that Trx may directly inactivate electrophilic drugs in a suicide inhibition motif (Herzig et al., 1999, Biochem. Pharm. 58: 217-225; Brandes et al., 1993, J. Biol. Chem. 268: 18411-18414). Both Trx and glutathione overexpression are signatures of drug resistant cells (Husbeck and Powis, 2002, Carcinogenesis 23: 1625-1630; Herzig et al., 1999, Biochem. Pharm. 58: 217-225; Rudin et al., 2003, Cancer Res. 63: 312-318; Butler et al., 2002, Proc. Natl. Acad. Sci. USA 99: 11700-11705). Indeed, Trx inhibition has been intensively examined as a means by which to remediate tumor cell resistance to classical chemotherapeutic agents (Moos et al., 2003, J. Biol. Chem. 278: 745-750; Naito et al., 1999, Int. J. Urology 6: 427-439). These considerations promote the notion that Trx takes part in the mechanism of activation for FK228 and that novel agents based on the FK228 structure will capitalize on a similar mode of activation.

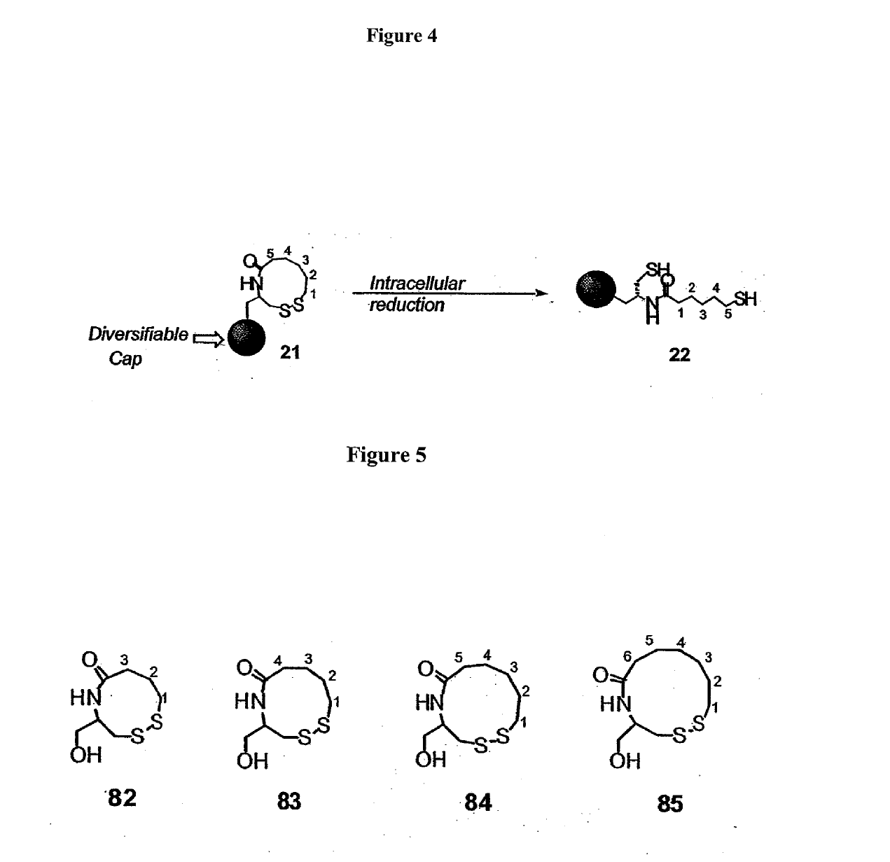

The inventors have developed the methodology described herein to generate readily diversifiable core structures useful in synthesizing FK228 analogs. Antiangiogenic activity, need for reductive activation, and scarcity of natural product drive the interest in formulating HDAC inhibitors structurally different from FK228 but sharing its unique mechanism of action. With the goal of generating and assaying a wide array of agents with the core structure represented in general by 21 (FIG. 4).

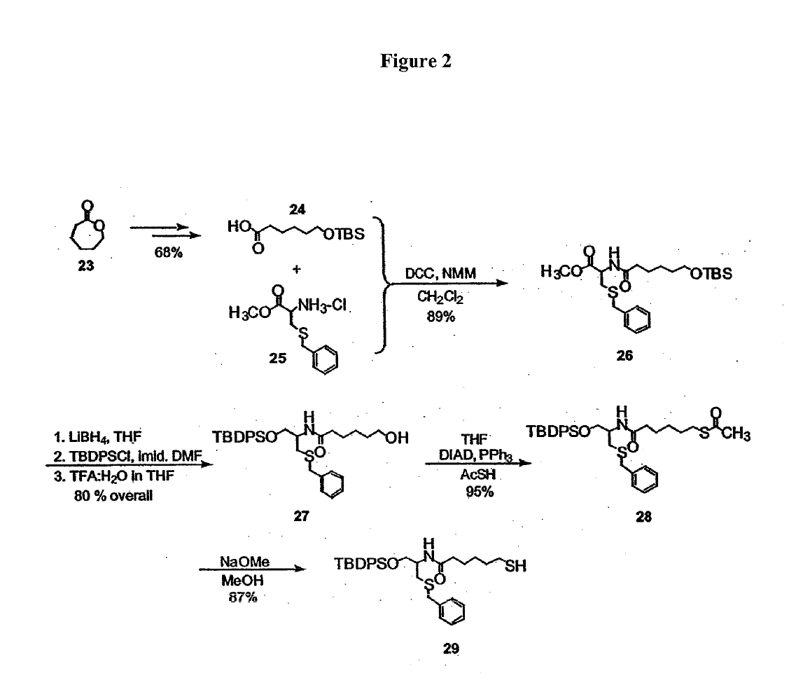

The inventors have initially conducted the synthesis shown by FIG. 2, and have subsequently assayed for HDAC inhibition of simple C5 linked thiols, which are precursors to cyclic disulfide analogs of FK228 as represented by 21. As shown in FIG. 2, one preferred pathway to compounds according to the invention is via hydrolysis of ε-caprolactone 23 using Nicolaou conditions followed by immediate alcohol silylation affords acid 24 which undergoes facile coupling to benzyl thioether 25 (Nicolaou et al., 1993, J. Am. Chem. Soc. 112: 3040-3055). The resulting amide 26 is readily converted to the hydroxymethyl analog 27, followed by tert-butyidiphenylsilyl (TBDPS) protection of the hydroxymethyl moiety and subsequent selective desilylation of the α-oxygen (Zhu et al., 2000, J. Chem. Soc. Perkin I 15 2305-2306). The resulting alcohol 27 is obtained in 80% yield from fully protected 26. Alcohol 27 is readily converted to the thioacetyl analog using methodology originally disclosed by Volante (Volante, 1981, Tet. Lett. 22: 3119-3122). Deacetylation of 28 is readily effected with sodium methoxide to afford free thiol 29. Importantly, 29 and a number of thiols generated in a similar way, are not readily susceptible to oxidation, which has important bearings upon subsequent molecular biology experiments. As well, attempts to deacetylate 28 with lithium aluminum hydride (LAH) lead to the TBDPS ether cleavage with concomitant thiol deacetylation to afford 33, shown below.

The synthetic intermediates 27, 28, and 29, also as shown in FIGS. 1 and 2 represent a number of related compounds bearing the C5 linker. In a preferred embodiment, these compounds 27, 28, 29 and 33 have either D/L stereo configuration. In another preferred embodiment, these compounds 27, 28, 29 and 33 have L stereo configuration. C5 linker-based molecules constructed include 31-34; all but 33 bear the same capping 1,8-naphthalimide structure as Scriptaid, a known HDAC inhibitor displaying roughly ⅓ the potency of TSA (Su et al., 2000, Cancer Res. 60: 3137-3142). Naphthalimide 31 is derived from LiBH₄ reduction of 26 followed by Mitsunobu coupling to 1,8-naphthalimide and subsequent generation of the acetylthiol in a fashion paralleling the conversion of 27 to 28. Alcohols 27, 33 and 34 serve as control compounds which allow a careful dissection of the roles played by the thiol, thioester, and simplistic cap groups of 28-32 as well as other agents based on the C5 linked thiols and thioesters. The results of biological studies provide direction in terms of linker lengths compatible with thiol-induced HDAC inhibition.

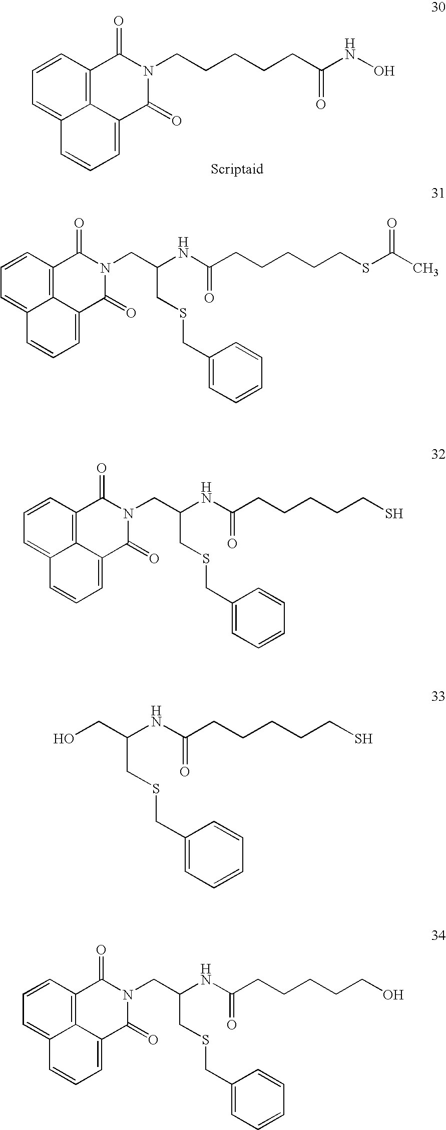

Assays to evaluate HDAC inhibition by FK228 analogs may capitalize on Western blotting methods and the wide assortment of Acetyl-Lys specific antibodies now available (Upstate Biotech Corp.). A general assay used in determining the HDAC inhibitor activity of compounds according to the invention are described below. Drosophila S2 cells can be cultured in Schneider's medium and grown as previously described by Wade and co-workers for periods ranging from 12 h to 96 h (Ballestar et al., 2001, Eur. J. Biochem. 268: 5397-5406). Cells may then be lysed and nuclear extracts prepared according to Thompson and co-workers (Steffan, 2001, Nature 413: 739-743). Lysates are then be subjected to differing concentrations of HDAC inhibitors and/or reductants (Barlow et al., 2001, Exp. Cell Res. 265: 90-103). The need to supplement these lysates with acetylated histones is then determined. Although the preferred method of acetyl Lys detection relies upon established Western blotting methods (Ballestar et al., 2001, Eur. J. Biochem. 268: 5397-5406), there are alternative isotope-based methods which have been quite successfully used with lysates from mouse melanoma B16/BL6 cells in the evaluation of cyclic tetrapeptide HDAC inhibitors (Komatsu et al., 2001, Cancer Res. 61: 4459-4466). The impact of inhibitors upon endogenous Drosophila HDACs such as HDACs1-4 and CG10899 may be evaluated (Chang et al., 2001, Proc. Nati. Acad. Sci. USA, 98: 9730-9735).

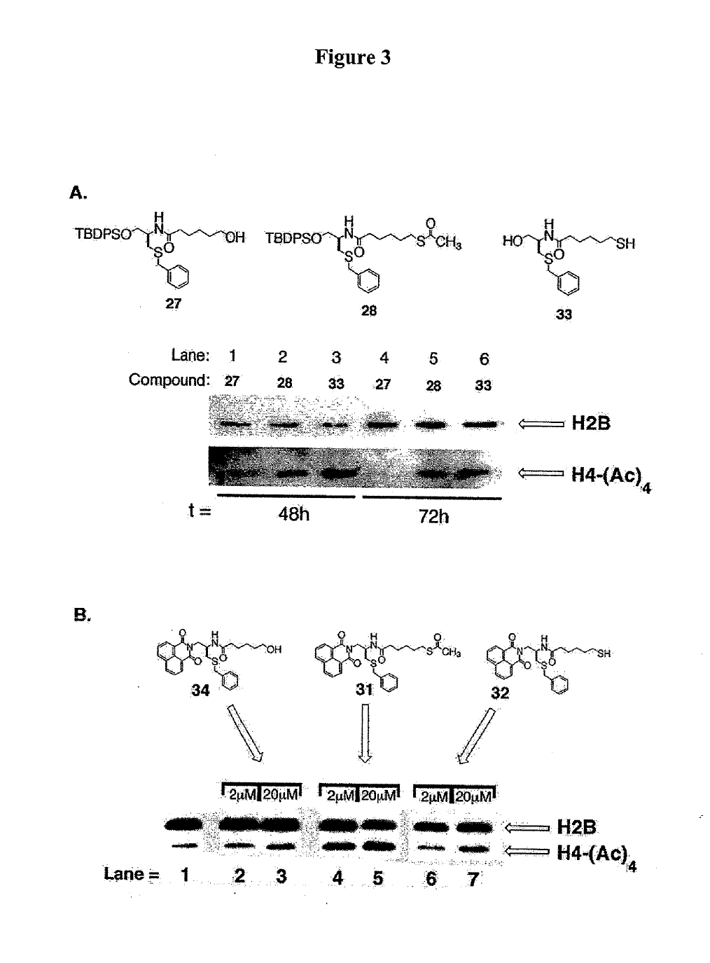

As shown in FIG. 3, examination of the bioactivity of disulfide precursors 27, 28 and 33 and 34, 31 and 32 using Drosophila S2 cells was performed by immunoprecipitation studies.

PanelA depicts Western blot analysis of histones isolated from Drosophila S2 cells treated with first generation cyclic disulfide precursors 27, 28 and 33. H2B detection was used to ensure equivalent protein loading to each well. Dark bottom bands most prominent in lanes 2,3, 5 and 6 indicate the enhanced abundance of tetra-AcLys H4, consistent with HDAC inhibition. Lanes 1 and 4 correspond to drosophila cells subjected to the “control” compound 27 which lacks an active site zinc binding terminal thiol or its precursor acetyl thiol. Cells in all reactions were incubated at ambient temperature in media that contained 20 μM of each respective synthetic agent. Incubations were conducted for either 48 or 72 hours prior to nuclei isolation and processing.

Panel B depicts Western blot analysis of histones isolated from Drosophila S2 cells treated with Scriptaid analogs 34, 31, and 32. H2B detection was used to ensure equivalent protein loading to each well. Dark bottom bands most prominent in lanes 4,5 and 7 indicate the enhanced abundance of tetra-AcLys H4, consistent with HDAC inhibition by 31 and 32. Lane 1 is a dioxane solvent control and concentrations of “drug” in cell medium are indicated above each lane. All cells were treated for a period of 48 hours at ambient temperature prior to nuclei isolation and processing.

The Western blot data indicate 28 and 33 to be potent cellular HDAC inhibitors at −20 μM concentration. Briefly, 2×10⁶ cells in 10 mL medium are brought to 2×10⁻⁵ M in synthetic intermediate and then allowed to incubate at ambient temperature for specified times. Cells were then pelleted, lysed under acid conditions and isolated histones processed using standard Western blotting methodology using Goat anti-H4 acetyl-Lys as primary antibody and horseradish peroxidase-based detection. Histone H2B was detected in each case in order to verify equal protein loadings for each lane. Lanes 1 and 4 correspond to histone isolated from cells treated with 27, which lacks any pharmacophore moiety but retains the synthetically useful TBDPS moiety which, from the biological perspective, serves as a “cap” group. Importantly, cells treated with 27 rendered little acetylated H4 as reflected by little or no chemiluminescent signal upon use of a horseradish peroxidase secondary antibody. Conversely, cells subjected to both 28 and 33 render, after lysis and processing, a significant amount of acetylated H4, which is consistent with HDAC inhibition. Although 33 is “activated” in the sense that the thiol terminus is capable of zinc binding once in the HDAC active site, 28 clearly is not. It is instructive to realize that thioacetyl analogs of two known HDAC inhibiting cyclic tetrapeptides are efficiently deacetylated by HDACs (Colletti et al., 2000). Most likely, enzymatic deacetylation of 28 is operative in these studies, allowing for two mechanisms of HDAC inhibition by 28—competition with acetyl-Lys for HDAC binding and simple metal binding following enzymatic processing. More importantly, these results support the concept that simple aliphatic thiols may mimic the biological activity and usefulness of activated FK228.

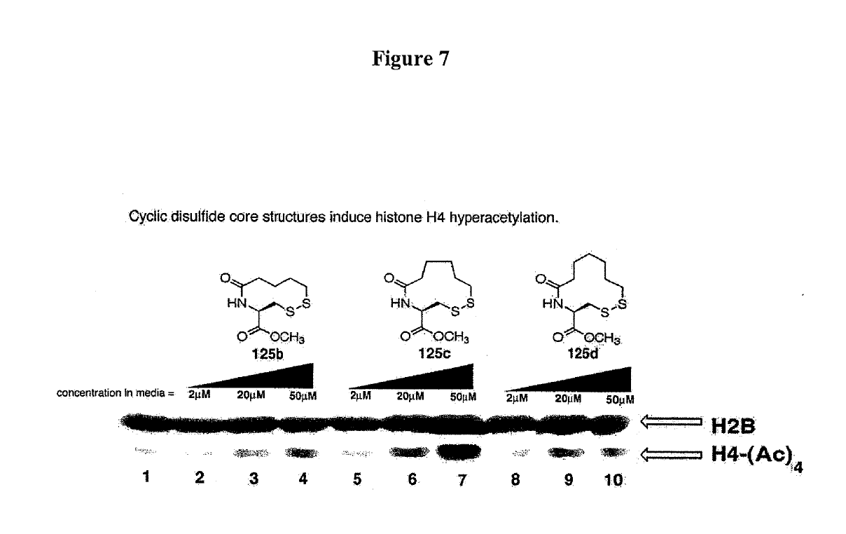

As shown in FIG. 7, Western blot analysis of histones isolated from Drosophila S2 cells treated with cyclic disulfides 125b-d. H2B detection was used to ensure equivalent protein loading to each well. Dark bottom band most prominent in lane 7 indicate the enhanced abundance of tetra-AcLys H4, consistent with HDAC inhibition by cyclic disulfide 125c which contains 5 methylene groups between the purported active site metal binding sulfur and the amide moiety proximal to the methyl ester. Lane 1 is a dioxane solvent control and concentrations of “drug” in cell medium are indicated above each lane. All cells were treated for a period of 48 hours at ambient temperature prior to nuclei isolation and processing.

Additionally, the reactivity of cyclic disulfide FK228 analogs may be analyzed with thioredoxin in cell free assays (this reductase is commercially available). The relevance of thioredoxin upregulation in tumor cells and its vital role in the production of glutathione as a major mechanism of drug resistance development indicates this enzyme is particularly relevant to the FK228 analogs inhibitors detailed herein (Butler et al. 2002, Proc. Natl. Acad. Sci. USA 99: 11700-11705; Shao et al., 2001, Cancer res. 61: 7333-7338; Becker et al., 2000, Eur. J. Biochem. 267: 6118-6125; Arner and Holmgren, 2000, Eur. J. Biochem. 267: 6102-6109; Kahlos et al., 2001, Int. J. Cancer 95: 198-204).

Drosophila, with its small well-defined genome and ability to generate mutants in a gene that has been cloned, provides an excellent system in which to study the actions of proposed inhibitors. The ability to ablate expression of specific genes using RNA interference (RNAi) provides further motivation for selecting Drosophila as the primary model in which to examine triggerable HDAC inhibitors (Paddison and Hannon, 2002, Cancer Cell 2002, 2, 17-23; Okajima and Irvine, 2002, Cell 111: 893-904; Tseng and Hariharan, 2002, Genetics 162: 229-243). Indeed, RNAi methods may play a role in our efforts to identify HDAC selective inhibitors, when significant difficulty in isolating purified HDACs is encountered.

Although cell-free systems have been extensively used to evaluate AMP kinase activity, DNA replication, chromatin remodeling/assembly and acetyltransferase inhibition (p300, CBP & P/CAF) in Drosophila, it is difficult to ensure that the precise transcriptional machinery in place within intact cells is not significantly perturbed following lysis (Rikhy et al., 2001, J. Neurosci. 22: 7478-7484; Krajewski, 1999, FEBS Lett. 452: 215-218; Krajewski, 2000, Mol. Gen. Genet. 263: 38-47; Pan and Hardie, 2002, Biochem J. 367: 179-186). As a complement to cell-free work, Drosophila S2 cells may be subjected to the proposed agents. Media is supplemented with varying concentrations of exogenous oxidoreductases (with accompanying cofactors) and cells grown for periods ranging from 12 to 96 h. Cell lysis is conducted and then histone analyses performed as previously described. Changes in histone acetylation status are ascertained via Western blots and these results contrasted with those derived using cell-free lysate. An alternative, albeit secondary approach is to generate S2 cells that overexpress deadhead (dhd), the gene which encodes the Drosophila homolog of human thioredoxin (Pellicena-Palle et al., 1997, Mech. Dev. 62: 61-65).

In generating redox triggered HDAC inhibitors, the inventors produced and characterized structurally simple analogs of FK228. The inventors' efforts on FK228 do not extend to close structural congeners of FK228 but rather, mechanistically related ones. Thus, FK228 analogs will all possess a similar pharmacophore (a terminal thiol or acetylated thiol) but will differ significantly in the identity of their “cap” structures. FK228-based molecules were examined from the perspective of possible cell-specific activation but also with an understanding of the importance for developing enzyme selective inhibitors by which to dissect the biochemistry of HDACs

The potent antiangiogenic and antimetastasis activity of FK228 results from HDAC1 inhibition by the depsipeptide natural product (Furumai et al., 2002, Cancer Res. 62: 4916-4921). As noted previously, the origin of FK228's intracellular activation is likely attributable to the actions of GSH and the major disulfide reductase thioredoxin. Significantly, enhanced expression levels of both reductants characterize many tumor cell types (Husbeck and Powis, 2002, Carcinogenesis 23: 1625-1630; Herzig et al., 1999, Biochem. Pharm. 58: 217-225; Rudin et al., 2003, Cancer res. 63: 312-318; Moos et al., 2003, J. Biol. Chem. 278: 745-750s). For instance, Gasdaska and co-workers have found elevated Trx mRNA levels in homogenates from non-small cell lung carcinomas. Trx is also overexpressed in gastric carcinomas; this overexpression has been linked to the development of cisplatin, mitomycin C, anthracycline and etoposide resistance (Becker et al., 2000, Eur. J. Biochem. 267: 6118-6125). It is therefore highly significant that Richon and co-workers have demonstrated that SAHA (5)) down regulates Trx in human primary breast and colon tumor tissues (Butler et al., 2002, Proc. Natl. Acad, Sci. USA 99: 11700-11705). It is unclear if SAHA will exert similar effects upon normal mammalian cells in which Trx plays a vital role in maintaining cellular redox levels compatible with DNA synthesis (via ribonucleotide reductase), transcription factor activity and the like.

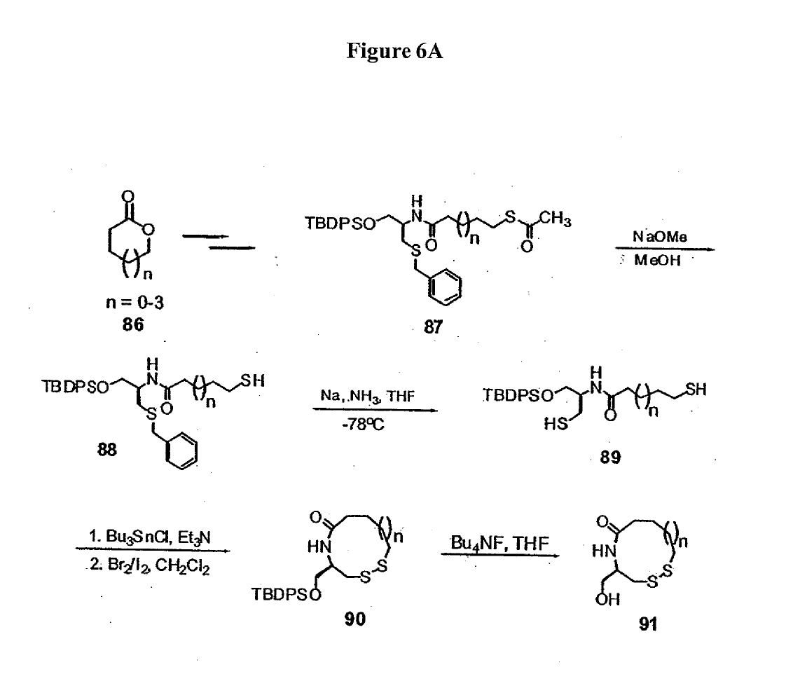

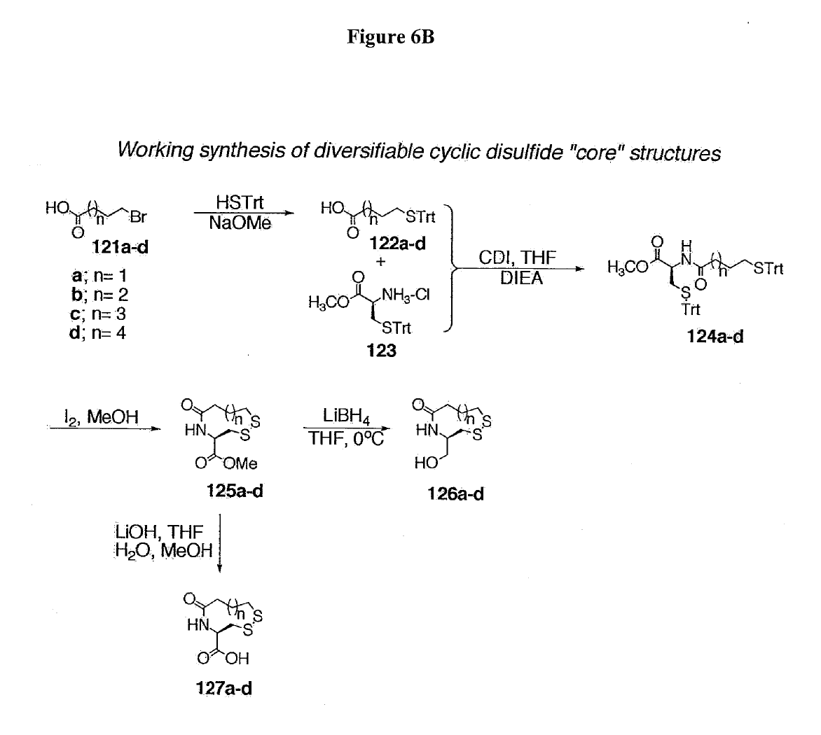

The scarcity of FK228 from natural sources and its very promising future promotes the interest in developing HDAC inhibitors that share FK228's novel mechanism of action but differ significantly in their level of synthetic demand (Li et al., 1996, J. Am. Chem. Soc. 118: 7237-7238). These endeavors also support the goal of developing bioreductively activated HDAC inhibitors. As shown in FIG. 5, in one preferred embodiment of the present invention, this invention teaches the synthesis of a library of cyclic disulfides (82-85) to which can easily be appended an assortment of different cap groups. As shown in FIG. 6a, lactones of the type 86 can be readily converted to their TBS silyl ether counterparts which are transformed to acetylthiols of generically depicted by 87. Deacetylation is readily accomplished with NaOMe although dissolving metal reduction of the S-benzyl moiety might proceed with concomitant deacetylation. In either event, generation of 89 is likely to proceed with high yields. Numerous opportunities exist for generation of the disulfide 90. Generation of bis(tri-n-butyltin) thiolates followed by subjection to either I₂ or Br₂ affords cyclic disulfides in very respectable yields. For example, ring sizes <7 are formed in 80% yield; for ring sizes of 8 or greater disulfides are generated in ˜50% yield (Harpp et al., 1986, Tet. Lett. 27: 441-444). This fares extremely well against alternative means of cyclic disulfide generation. Alternatively, conditions reported by Zoller and co-workers can be used (Zoller et al., 2000, Tet. Lett. 41: 9989-9992). Reaction of 86 with dimethylsulfide and N-chlorosuccinimide affords an activated dimethylsulfonium which readily reacts with S-benzyl thioethers to give the debenzylated disulfide. This chemistry is applicable to compounds with ring sizes ranging from 6 to 14 and is compatible with peptidic substrates. Following disulfide generation, desilylation will be performed thus rendering the hydroxymethyl moiety again, as a handle by which to attach diverse cap structures.

In a more preferred embodiment, as shown in FIG. 6b, the present invention also teaches a highly efficient method for the construction of cyclic disulfides based on the synthesis of differentially protected dithiols. This method, inspired by Simon's original total synthesis of FK228, calls for production of 124a-d (two steps from commercially available materials) followed by 12-mediated disulfide installation. Importantly, bromoacids of form 121 bearing 3,4,5, and 6 methylene units are commercially available and the inventors have already shown that the chemistry in FIG. 6b works very efficiently to generate substances like 124 following carbonyidiimidazole (CDI)-mediated amide bond formation with S-Trt cysteine methyl ester 123. Highlighted in FIG. 6b, the ditritylated methyl esters of form 124 undergo very clean conversion to macrocycles of kind 125. The inventors have successfully produced agents 125b-d using this high yielding method. Further, the inventors have found that 125b is amenable to LiBH4 reduction to render 126b; this chemistry should be applicable to materials 126a, 126c and 126d as well. Importantly, the hydroxymethyl group of all 126 agents provides one of two possible handles by which to attach appropriate capping groups of interest. In addition to the reduction of the methyl ester moiety in 125, the inventors have also successfully saponified methyl esters 125b-d to afford acids 127b-d; the inventors believe 127a will also be achievable using this base-catalyzed hydrolysis. Agents of the type 127 are important since the carboxylic acid is readily coupled to a wide assortment of amine-bearing cap groups. For the formation of amide-linked cyclic disulfides the inventors have found CDI to be a very effective agent. The panel of amino bearing cap groups will facilitate rapid construction and assaying for a wide array of cyclic disulfides with a high likelihood for HDAC inhibitory activity.

The extreme limitations on structural knowledge of HDAC:inhibitor interactions have led to the examination of broadly different cap structures (Jung et al., 1999, J. Med. Chem. 42: 4669-4679; Lavoie et al., 2001, Bioorg. Med. Chem. Lett. 11: 2847-2850; Uesato et al., 2002, Bioorg. Med. Chem. Lett. 12: 1347-1349). The one constant criterion is that of relatively lipophilic moieties as the major constituents of cap structure (Breslow et al., 2000, Helv. Chim. Acta 83: 1685-1692; Marks et al., 2001, Curr. Op. Oncol. 13: 477-483; Taunton et al., 1996; Colletti et al., 2000; Jung et al., 1999, J. Med. Chem. 42: 4669-4679; Lavoie et al., 2001, Bioorg. Med. Chem. Lett. 11: 2847-2850; Uesato et al., 2002, Bioorg. Med. Chem. Lett. 12: 1347-1349). There was originally noted in early studies of trapoxin, apicidin and other cyclic tetrapeptides where IC₅₀ values were positively impacted by the extent of hydrophobicity within cap structures. On the strength of findings at Abbott and MethylGene the inventors are attaching to cyclic disulfides a number of different commercially available pthalimides and polycyclic phenols. Hydroxamates 93 and 94 have been developed at Abbott (Frey et al., 2001, Bioorg. Med. Chem. Lett. 12: 3443-3447; Curtin et al., 2002, Bioorg. Med. Chem. Lett. 12: 1919-1923)as highly effective inhibitors of HDAC1 whereas MethylGene (Woo et al., 2002, J. Med. Chem. 45: 2877-2885) has identified 95 as just one out of a panel of 18 materials with sub-100 nM IC₅₀ values against HDAC1. These agents are increasingly representative of very potent, simply constructed HDAC inhibitors devoid of cell selectivity manifolds (Jung et al., 1999, J. Med. Chem. 42: 4669-4679; Lavoie et al., 2001, Bioorg. Med. Chem. Lett. 11: 2847-2850; Uesato et al., 2002, Bioorg. Med. Chem. Lett. 12: 1347-1349; Frey et al., 2001, Bioorg. Med. Chem. Lett. 12: 3443-3447; Curtin et al., 2002, Bioorg. Med. Chem. Lett. 12: 1919-1923; Woo et al., 2002, J. Med. Chem. 45: 2877-2885).

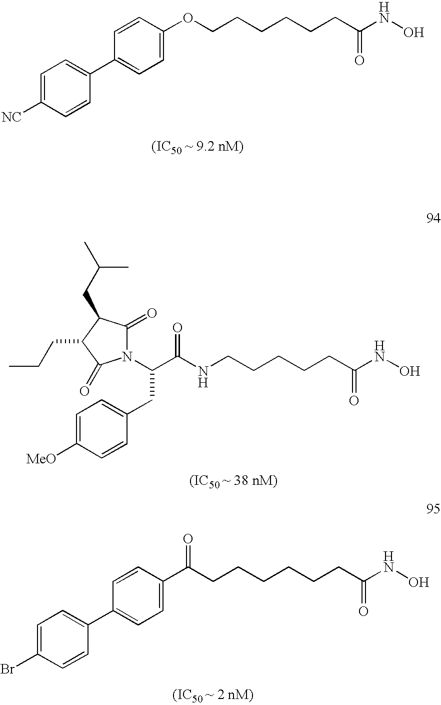

The hydroxymethyl moiety of the proposed cyclic disulfides lends itself well to Mitsunobu condensation with an array of nucleophilic cap groups (Hanessian et al., 2002, Tet. Lett. 43: 1995-1998; Tsai et al., 2000, Tet. Lett. 41: 9499-9503; Florez-Alvarez et al., 2002, Tet. Lett. 43: 171-174; Abdaoui et al., 1996, Tet. Lett. 37: 5695-5698). Although small amounts of the oxazoline are often formed, the intended intermolecular coupling proceeds with good efficiency in the case of a hydroxymethyl-bearing precursor to 84 (Harpp et al., 1986, Tet. Lett. 27: 441-444). Oxazoline formation constitutes ˜10-20% of the obtained product from such couplings. The cyclic nature of 79-82 abrogates oxazoline formation, instead favoring the desired “capped” products.

The compatibility of disulfides with Mitsunobu coupling conditions is well known (Florez-Alvarez et al., 2002, Tet. Lett. 43: 171-174; Abdaoui et al., 1996, Tet. Lett. 37: 5695-5698). Therefore, the attention is focused on the condensation of 79-82 with cap units shown below or equivalent such units.

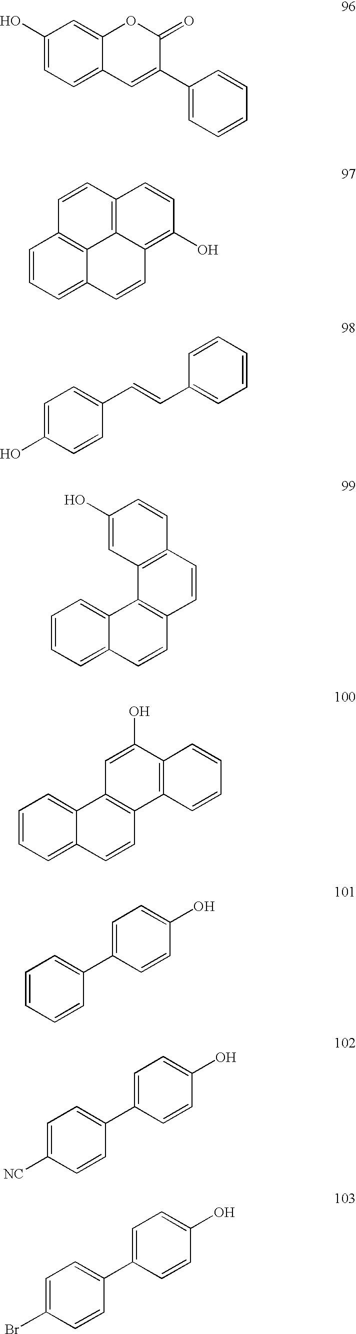

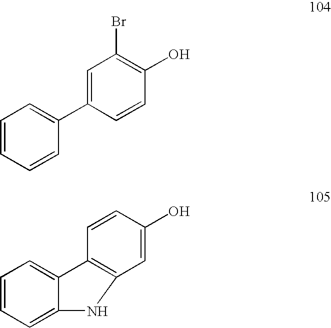

In addition to the condensation of disulfides with phenol moieties 96-105, structures in which the cap derives from the panel of commercially available naphthalimides are generated. 108 is readily amenable to hydroxymethyl conjugation en route to 31, 32, and 34. This finding should extend well to the panel below.

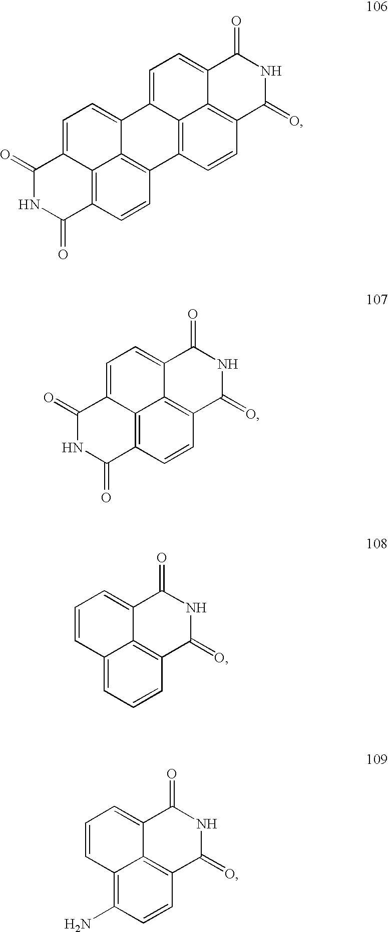

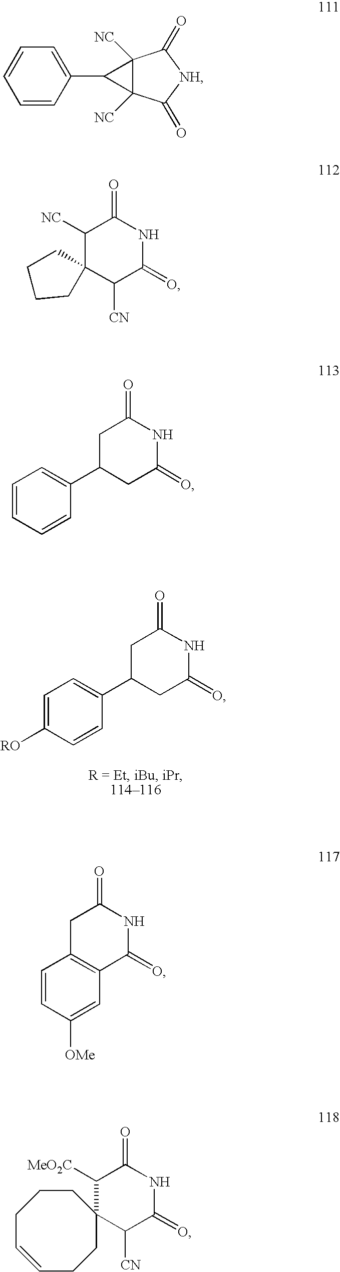

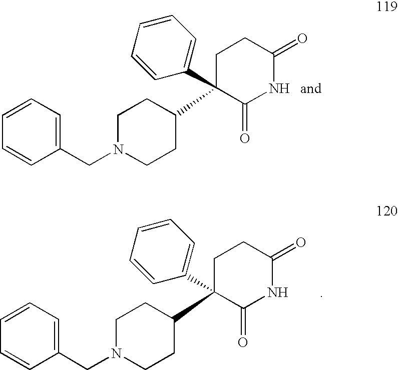

Also, the following primary amine cap groups may also be incorporated via amide formation:

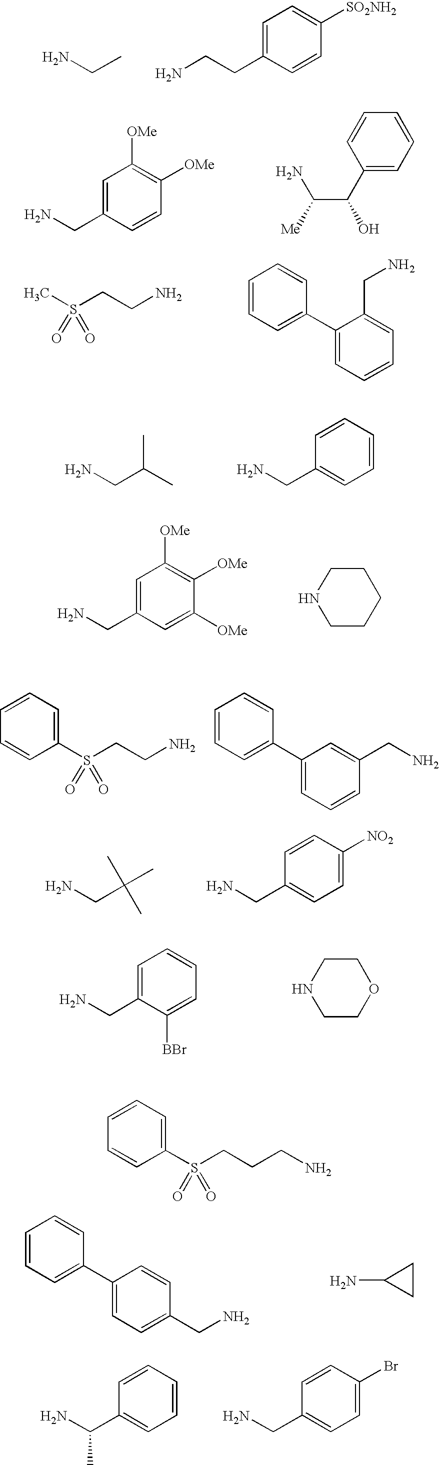

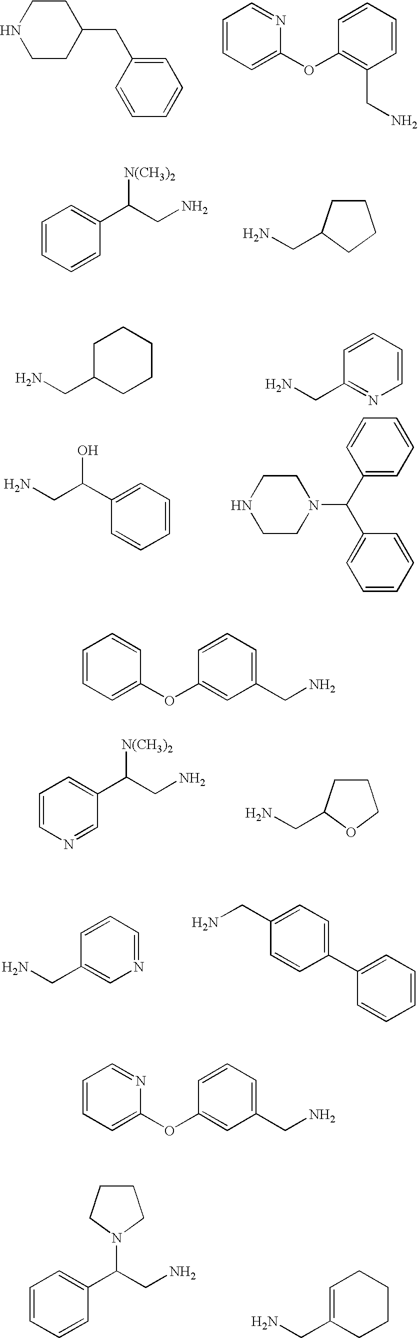

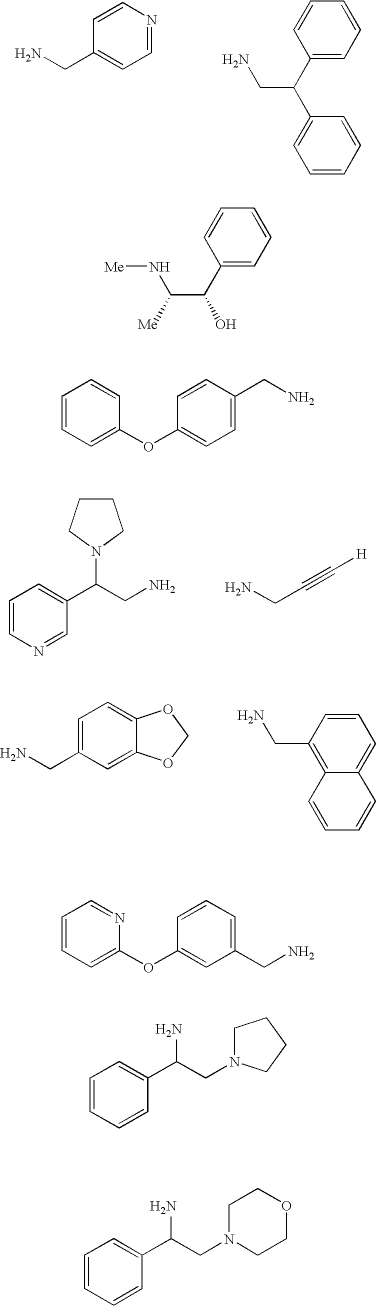

As used herein, a “targeting agent” may be any substance (such as a compound) that, when associated with an analog of FK228 enhances the local concentration of the analog at a target tissue.

Targeting agents include, in addition to capping structures described elsewhere, antibodies or fragments thereof, receptors, ligands and other molecules that bind to cells of, or in the vicinity of, the target tissue. Known targeting agents include serum hormones, antibodies against cell surface antigens, lectins, adhesion molecules, tumor cell surface binding ligands, steroids, cholesterol, lymphokines, fibrinolytic enzymes and those drugs and proteins that bind to a desired target site. Among the many monoclonal antibodies that may serve as targeting agents are anti-TAC, or other interleukin-2 receptor antibodies; 9.2.27 and NR-ML-05, reactive with the 250 kilodalton human melanoma-associated proteoglycan; and NR-LU-10, reactive with a pancarcinoma glycoprotein. An antibody targeting agent may be an intact (whole) molecule, a fragment thereof, or a functional equivalent thereof. Examples of antibody fragments are F(ab′)₂, -Fab′, Fab and F[v] fragments, which may be produced by conventional methods or by genetic or protein engineering. Linkage is generally covalent and may be achieved by, for example, direct condensation or other reactions, or by way of bi- or multi-functional linkers. Within other embodiments, it may also be possible to target a polynucleotide encoding a modulating agent to a target tissue, thereby increasing the local concentration of modulating agent, e.g. thioredoxin, thioredoxin reductase or other reductive enzymes. Such targeting may be achieved using well known techniques, including retroviral and adenoviral infection, as described above.

In another aspect, the present invention provides pharmaceutically acceptable compositions which comprise a therapeutically-effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream patches or foam.

The phrase “therapeutically-effective amount” as used herein means that amount of a compound, material, or composition comprising a deacetylase inhibitor of the present invention which is effective for producing some desired therapeutic effect by inhibiting histone deacetylation in at least a sub-population of cells in an animal and thereby blocking the biological consequences of that event in the treated cells, at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject deacetylase inhibitor agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, certain embodiments of the present deacetylase inhibitors may contain a basic functional group, such as amino or alkyl amino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J Pharm. Sci. 66:1-19)

In other cases, the deacetylase inhibitory compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the deacetylase inhibitor which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a deacetylase inhibitor of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A deacetylase inhibitor of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered deacetylase inhibitor moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the deacetylase inhibitors of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corm, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active deacetylase inhibitor, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active deacetylase inhibitor.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a deacetylase inhibitor of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active deacetylase inhibitor of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the deacetylase inhibitor in the proper medium. Absorption enhancers can also be used to increase the flux of the deacetylase inhibitor across the skin. The rate of such flux can be controlled by either providing a rate controlling membrane or dispersing the deacetylase inhibitor in a polymer matrix or gel.

Ophthalmic formulations, eye ointments, powders, solutions and the like, are also contemplated as being within the scope of this invention.

Pharmaceutical compositions of this invention suitable for parenteral administration comprise one or more deacetylase inhibitors of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium cWoride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject deacetylase inhibitors in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The deacetylase inhibitor may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular deacetylase inhibitor employed, or the ester, salt or amide thereof, the route of administration, the time of administration, the rate of excretion of the particular compound being employed, the duration of the treatment, other drugs, compounds and/or materials used in combination with the particular deacetylase inhibitor employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

The compounds of the present invention are likely to play an important role in the modulation of cellular proliferation. There are a wide variety of pathological cell proliferative conditions for which therapeutics of the present invention may be used in treatment. For instance, such agents can provide therapeutic benefits where the general strategy being the inhibition of an anomalous cell proliferation. Diseases that might benefit from this methodology include, but are not limited to various cancers and leukemias, psoriasis, bone diseases, fibroproliferative disorders such as involving connective tissues, atherosclerosis and other smooth muscle proliferative disorders, as well as chronic inflammation.

In addition to proliferative disorders, the present invention contemplates the use of therapeutics for the treatment of differentiative disorders which result from, for example, de-differentiation of tissue which may (optionally) be accompanied by abortive reentry into mitosis, e.g. apoptosis. Such degenerative disorders include chronic neurodegenerative diseases of the nervous system, including Alzheimer's disease, Parkinson's disease, Huntington's chorea, amylotrophic lateral sclerosis and the like, as well as spinocerebellar degenerations. Other differentiative disorders include, for example, disorders associated with connective tissue, such as may occur due to de-differentiation of chondrocytes or osteocytes, as well as vascular disorders which involve de-differentiation of endothelial tissue and smooth muscle cells, gastric ulcers characterized by degenerative changes in glandular cells, and renal conditions marked by failure to differentiate, e.g. Wilm's tumors.

It will also be apparent that, by transient use of modulators of histone deacetylase activities, in vivo reformation of tissue can be accomplished, e.g. in the development and maintenance of organs. By controlling the proliferative and differentiative potential for different cells, the subject therapeutics can be used to reform injured tissue, or to improve grafting and morphology of transplanted tissue. For instance, antagonists and agonists can be employed in a differential manner to regulate different stages of organ repair after physical, chemical or pathological insult. For example, such regimens can be utilized in repair of cartilage, increasing bone density, liver repair subsequent to a partial hepatectomy, or to promote regeneration of lung tissue in the treatment of emphysema. The present method is also applicable to cell culture techniques.

Those skilled in the art will recognize, or be able to ascertain using no more then routine experimentation, numerous equivalents to the specific polypeptides, nucleic acids, methods, assays and reagents described herein. Such equivalents are considered to be within the scope of this invention and covered by the following claims. All publications, patents, and patent applications cited herein are hereby incorporated by reference in their entirety for all purposes.

### V. REFERENCES

(1) Abdaoui, M.; Dewynter, G.; Montero, J.-L. “Expedient Synthesis of 2-Chloroethyinitrososulfamides (CENS) via the Decarboxylative Reopening of Sulfamoyloxazolidinones” Tet. Lett. 1996, 37, 5695-5698.
(2) Ahmad, K., Henikoff, S. “Epigenetic Consequences of Nucleosome Dynamics”, Cell, 2002, 111, 281-284.
(3) Andrews, K. T., Walduck, A., Kelso, M. J., Fairlie, D. P., Saul, A., Parsons, P. G. “Anti-malarial effect of histone deacetylase inhibitors and mammalian tumour differentiating agents”, lnt. J. Parasit. 2000, 30, 761-768.
(4) Archer, S. Y., Hodin, R. A. “Histone Acetylation and Cancer”, Curr. Op. Gen. and Dev. 1999, 9, 171.
(5) Arner, E. S. J., Holmgren, A. “Physiological functions of thioredoxin and thioredoxin reductase” Eur. J. Biochem. 2000, 267, 6102-6109.
(6) Baldwin, J. E., Adlington, R. M., Godfrey, R. A., Pate I, V. K. “Stereospecific Synthesis of Chlamydocin” Tetrahedron 1993, 49, 7837-7856.
(7) Ballestar, E., Pile, L. A., Wassarman, D. A., Wolffe, A. P.; Wade, P. A. “A Drosophila MBD family member is a transcriptional corepressor associated with specific genes” Eur. J. Biochem. 2001, 268, 5397-5406.
(8) Bathgate, A.; Malpass, J. R. “A Simple Approach to Nortropane and Nortrop-6-ene Derivatives”, Tet. Lett. 1987, 28, 5937-5940.
(9) Barlow, A. L.; van Drunen, C. M.; Johnson, C. A.; Tweedie, S.; Bird, A.; Turner, B. M. “dSIR2 and dHDAC6: Two Novel, Inhibitor-Resistant Deacetylases in Drosophila melanogaster” Exp. Cell Res. 2001, 265, 90-103.
(10) Becker, K.; Gromer, S.; Schirmer, R. H.; Muller, S. “Thioredoxin reductase as a pathophysiological factor and drug target” Eur. J. Biochem. 2000, 267, 6118-6125.
(11) Bedalov, A.; Gatbonton, T.; Irvine, W. P.; Gottschling, D. E.; Simon, J. A. “Identification of a Small Molecule Inhibitor of Sir2p “Proc. Natl. Acad. Sci. USA 2001, 98, 15113-15118.
(12) Bendavid, A.; Burns, C. J.; Field, L. D.; Hashimoto, K.; Ridley, D. D.; Samankumara Sandanayake, K. R. A.; Wieczorek, L. “Solution-and Solid-Phase Synthesis of Components for Tethered Bilayer Membranes” J. Org. Chem. 2001, 66, 3709-3716.
(13) Berger, S. L.; Gelsenfeld, G. “Chromatin Goes Global”, Mol. Cell. 2001, 8, 263-268.
(14) Bernardi, E.; Cros, S.; Viallefont, P.; Lazaro, R. “Synthesis of new cytotoxic cyclopeptide analogs of Chlamydocin” Bull. Soc. Chem. Fr. 1994, 131, 944-948.
(15) Berst, F.; Ladlow, M.; Holmes, A. B. “Solid-phase synthesis of apicidin A and a cyclic tetrapeptoid analogue” Chem. Comm. 2002, 508-509.
(16) Besser, D.; Olender, R.; Rosenfeld, R.; Arad, O.; Reissman, S. “Study on the cyclization tendency of backbone cyclic tetrapeptides” J. Peptide Res. 2000, 56, 337-345.
(17) Blagosklonny, M. V. “Hypoxia-inducible factor: Achilles' heel of antiangiogenic cancer therapy” Intl. J. Oncol. 2001, 19, 257-262.
(18) Brandes, H. K.; Larimer, F. W.; Geck, M. K.; Stringer, C. D.; Schuerman, P.; Hartman, F. C. “Direct Identification of the primary nucleophile of thioredoxin” J. BioL Chem. 1993, 268, 18411-18414.
(19) Breslow, R.; Belvedere, S.; Gershell, L. “Development of Cytodifferentiating Agents for Cancer Chemotherapy”, Helv. Chim. Acta 2000, 83, 1685-1692.
(20) Butler, L. M.; Zhou, X.; Xu, W.-S.; Scher, H. I.; Rifkind, R. A.; Marks, P. A.; Richon, V. M. “The histone deacetylase inhibitor SAHA arrests cancer cell growth, up-regulates thioredoxin-binding protein-2, and down-regulates thioredoxin” Proc. Natl. Acad. Sci. USA 2002, 99, 11700-11705.
(21) Cairns, B. R. “Emerging Roles for Chromatin Remodeling in Cancer Biology”, TIBS 2001, 11, S15-S21.
(22) Chang, Y.-L.; Peng, Y.-H.; Pan, I.-C.; Sun, D.-S.; King, B.; Huang, D.-H. “Essential role of Drosophila Hdac1in homeotic gene silencing” Proc. Natl. Acad. Sci. USA 2001, 98, 9730-9735.
(23) Cheung, P.; Allis, C. D.; Sassone-Corsi, P. “i Signaling to Chromatin through Histone Modifications”Cell 2000, 103, 263-271.
(24) Colletti, S. L.; Li, C.; Fisher, M. H.; Wyvratt, M. J.; Meinke, P. T. “Tryptophan-replacement and indole-modified apicidins: synthesis of potent and selective antiprotozoal agents” Tet. Lett. 2000, 41, 7825-7829.
(25) Colletti, S. L.; Myers, R. W.; Darkin-Rattray, S.; Schmatz, D. M.; Fisher, M. H.; Wyvratt, M. J.; Meinke, P. T. “Design and Synthesis of Histone Deacetylase Inhibitors: The development of apicidin transition state analogs”, Tet. Lett. 2000, 41, 7837-7841.
(26) Crevel, G.; Cotterill, S. “DNA Replication in Cell-free extracts from Drosophila melanogaster” EMBO J. 1991, 10, 4361-4369.
(27) Curtin, M. L.; Garland, R. B.; Heyman, R.; Frey, R. R.; Michaelides, M. R.; Li, J.; Pease, L. J.; Glaser, K. B.; Marcotte, P. A.; Davidsen, S. K. “Succinimide Hydroxamic Acids as Potent Inhibitors of Histone Deacetylase (HDAC)” Bioorg. Med. Chem. Lett. 2002, 12, 2919-1923.
(28) Cvetkovic, D.; Movsas, B.; Dicker, A. P.; Hanlon, A. L.; Greenberg, R. E.; Chapman, J. D.; Hanks, G. E.; Tricoli, J. V., “Increased Hypoxia Correlates with Increased Expression of the Angiogenesis Marker Vascular Endothelial Growth Factor in Human Prostate Cancer” Urology 2001, 57, 821-825.
(29) Colletti, S. L.; Myers, R. W.; Darkin-Rattray, S.; Schmatz, D. M.; Fisher, M. H.; Wyvratt, M. J.; Meinke, P. T. “Design and Synthesis of Histone Deacetylase Inhibitors: The development of apicidin transition state analogs”, Tet. Lett. 2000, 41, 7837-7841.
(30) Denu, J. M. “Linking Chromatin Function with Metabolic Networks: Sir2 family of NAD⁺-Dependent Deacetylases” TIBS 2003, 28, 41-48.
(31) Dieter, H. “Histone acetylation modifiers in the pathogenesis of malignant disease”, Mol. Med. 2000, 6, 623-644.
(32) Dutnall, R. N.; Pillus, L. “Deciphering NAD-Dependent Deacetylases” Cell, 2001, 105, 161-164.
(33) Elaut, G.; Torok, G.; Vinken, M.; Laus, G.; Papeleu, P.; Tourwe, D. Rogiers, V. “Major Phase I Biotransformation Pathways of Trichostatin A in Rat Hepatocytes and in Rat and Human Liver Microsomes” Drug. Metabolism and Disposition 2002, 30, 1320-1328.
(34) Finnin, M. S.; Donigian, J. R.; Cohen, A.; Richon, V. M.; Rifkind, R. A.; Marks, P. S.; Breslow, R.; Pavietich, N. P, “Structures of a histone deacetylase homologue bound to the TSA and SAHA inhibitors” Nature 1999, 401, 188.
(35) Florez-Alvarez, J.; Brocklehurst, K.; Gallacher, G.; Resmini, M. “Synthesis of an aminoalcohol hapten for the generation of catalytic antibodies” Tet. Lett. 2002, 43, 171-174.
(36) Frey, R. R.; Wada, C. K.; Garland, R. B.; Curtin, M. L.; Michaelides, M. R.; Li, J.; Pease, L. J.; Glaser, K. B.; Marcotte, P. A.; Bouska, J.J.; Murphy, S. S.; Davidsen, S. K. “Trifluoromethyl Ketones as Inhibitors of Histone Deacetylase” Bioorg. Med. Chem. Lett. 2001, 12, 3443-3447.
(37) Furumai, R.; Komatsu, Y.; Nishino, N.; Khochbin, S.; Yoshida, M.; Horinuchi, S. “Potent Histone Deacetylase Inhibitors Built from Trichostatin A and Cyclic Tetrapeptide Antibiotics Including Trapoxin” Proc. Natl. Acad. Sci. USA 2001, 98, 87-92.
(38) Furumai, R.; Matsuyama, A.; Kobashi, N.; Lee, K.-L.; Nishiyama, M.; Nakajima, H.; Tanaka, A.; Komatsu, Y.; Nishino, N.; Yoshida, M.; Horinouchi, S. “FK228 (Depsipeptide) as a Natural Prodrug That Inhibits Class I Histone Deacetylases” Cancer Res. 2002, 62, 4916-4921.
(39) Gasdaska, P.; Oblong, J. E.; Cotgreave, I. A.; Powis, G. “The predicted amino acid sequence of human thioredoxin is identical to that of the autocrine growth factor human adult T-cell derived factor (ADF): thioredoxin mRNA is elevated in some human tumors”, Biochim. Biophys. Acta. 1994, 1218, 292-296.
(40) Grozinger, C. M.; Schreiber, S. L. “Deacetylase Enzymes: Biological Functions and the Use of Small-Molecule Inhibitors” Chem. Biol. 2002, 9, 3-16.
(41) Hanessian, S.; van Otterlo, W. A. L.; Nilsson, I.; Bauer, U. “Stereocontrolled synthesis of a prototype library of enantiopure 2,4-disubstituted 4-aryl-6-piperidinones and piperidines” Tet. Lett. 2002, 43, 1995-1998.
(42) Harpp, D. N.; Bodzay, S. J.; Aida, T.; Chan, T. H. “High Yield Preparation of Cyclic Disulfides Using Alkyltin Th iolates” Tet. Lett. 1986, 27, 441-444.
(43) Hassan, A. H.; Prochasson, P.; Neely, K. E.; Galasinski, S. C.; Chandy, M.; Carrozz, M. J.; Workman, J. L. “Function and Selectivity of Bromodomains in Anchoring Chromatin-Modifying Complexes to Promoter Nucleosomes”, Cell 2002, 111, 369-379.
(44) Hassig, C. A.; Schreiber, S. L. “Nuclear Histone Acetylases and Deacetylases and Transcriptional Regulation: HATs off to HDACs”, Curr. Op. in Chemical Biology,1997, 1, 300.
(45) Herzig, M. C. S.; Arnett, B.; MacDonald, J. R.; Woynarowski, J. M. “Drug uptake and cellular targets of hydroxymethylacylfulvene (HMAF)”, Biochem. Pharm. 1999, 58,217-225;
(46) Hockel, M.; Vaupel. P. “Biological consequences of tumor hypoxia” Seminars in Oncology 2001, 28, 36-41.
(47) Hoffmann, K.; Heltweg, B.; Jung, M. “Improvement and validation of the fluorescence-based histone deacetylase assay using an internal standard” Arch. Pharm. (Weinhem, Ger.) 2001, 334, 248-252.
(48) Hoffmann, K.; Soil, R. M.; Beck-Sickinger, A. G.; Jung, M. “Fluorescence-Labeled Octapeptides as Substrates for Histone Deacetylase” Biooconjugate Chem. 2001, 12, 51-55.
(49) Hung, D. T.; Jamison, T. F.; Schreiber, S. L., “Understanding and Controlling the Cell Cycle with Natural Products”, Chem. Biol. 1996, 3, 623.
(50) Husbeck B.; Powis, G. “The redox protein thioredoxin-1 regulates the constitutive and inducible expression of the estrogen metabolizing cytochromes P4501B1 and 1A1 in MCF-7 human breast cancer cells” Carcinogenesis 2002, 23, 1625-1630.
(51) Huss, W. J.; Hanrahan, C. F.; Barrios, R. J.; Simons, J. W.; Greenberg, N. M., “Angiogenesis and Prostate Cancer Identification of a Molecular Progression Switch”, Cancer Res. 2001, 61, 2736-2743.
(52) Jaffar, M.; Naylor, M. A.; Robertson, N.; Stratford, I. J. “Targeting hypoxia with a new generation of indolequinones” Anti-Cancer Drug Design 1998, 13, 593-609.
(53) Jacquier, R.; Lazaro, R.; Raniriseheno, H.; Viallefont, Ph. “Synthesis of HC toxin and related cyclopeptides containing the (L-Ala-D-Ala-L-Ada-D-Pro) sequence”, lnt. J. Peptide Protein Res. 1987, 30, 22-32.
(54) Jacquier, R.; Lazaro, R.; Raniriseheno, H.; Vial lefont, Ph., “Stereoselective Synthesis of HC-Toxin and Its (9R) Epoxide Epimer”, Tet. Lett. 1986, 27, 4735.
(55) Jones, P. L.; Veenstra, G. J.; Wade, P. A.; Vermaak, D.; Kass, S. U.; Landsberger, N.; Strouboulis, J.; Wolffe, A. P. “Methylated DNA and MeCP2 recruit histone deacetylase to repress transcription”, Nat. Genet. 1998 19, 187-191.
(56) Jung, M.; Brosch, G.; Kolle, D.; Scherf, H.; Gerhauser, C.; Loidl, P. “Amide Analogues of Trichostatin as Inhibitors of Histone Deacetylase and Inducers of Terminal Cell Differentiation” J. Med. Chem. 1999, 42, 4669-4679.
(57) Kahlos, K.; Soini, Y.; Saily, M.; Koistinen, P.; Kakko, S.; Paakko, P.; Holmgren, A.; Kinnula, V. L. “Up-regulation of thioredoxin and thioredoxin reductase in human malignant pleural mesothelioma” Int. J. Cancer, 2001, 95, 198-204.
(58) Keana, J. F. W.; Heo, G. S.; Gaughon, G. T. “Stereospecific Synthesis of Difunctionalized 2,5,-Disubstituted cis-2,5-Dimethylpyrrolidine (Azethoxyl) Nitroxides by Oxidative Cleavage of Protected 8-Azabicyclo[3.2.1]-octane Precursors”, J. Org. Chem. 1985, 50, 2346-2351.
(59) Kelly, W. K.; O'Connor, O. A.; Marks, P. A. “Histone Deacetylase Inhibitors: From Target to Clinical Trials”, Expert Op. Invest. Drugs 2002, 11, 1695-1713.
(60) Kemp, D. S.; Rothman, J. H. “Synthesis and Analysis of a Macrocyclic Triproline-Derived Template Containing a Local Conformational Constraint” Tet. Lett., 1995,36,4019-4022.
(61) Kemp, D. S.; Rothman, J. H. “Efficient Helix Nucleation by a Macrocyclic Triproline-Derived Template”, Tet. Lett. 1995, 36, 4023-4026.
(62) Khochbin, S.; Verdel, A.; Lemercier, C.; Seighneurin-Berny, D. “Functional Significance of Histone Deacetylase Diversity”, Curr. Opin. Gen. & Dev. 2001, 11, 162-166.
(63) Kijima, M.; Yoshida, M.; Sugita, K.; Horinouchi, S.; Beppu, T., “Trapoxin, an Antitumor Cyclic Tetrapeptide, is an Irreversible Inhibitor of Mammalian Histone Deacetylase” J. Biol. Chem. 1993, 268, 22429.
(64) Kim, K,-W., et. al. “Histone deacetylases induce angiogenesis by negative regulation of tumor suppressor Genes” Nature Medicine, 2001, 7, 437-443.
(65) Kitamura, S.; Takekawa, K.; Sugihara, K.; Tatsumi, K.; Ohta, S. “Pseudoenzymatic reduction of N-hydroxy-2-acetylaminofluorene to 2-acetylaminofluorene mediated by cytochrome P450” Carcinogenesis, 1999, 20, 347-350.
(66) Klar, A. J. S.; Srikantha, T.; Soil, D. R. “A Histone Deacetylation Inhibitor and Mutant Promote Colony-Type Switching of the Human Pathogen Candida albicans”, Genetics 2001, 158, 919-924.
(67) Klohs, W. D.; Hamby, J. M. “Antiangiogenic Agents” Curr. Op. Biotech. 1999, 10, 544-549.
(68) Komatsu, Y.; Tomizaki, T.; Tsukamoto, M.; Kato, T.; Nishino, N.; Sato, S.; Yamori, T.; Tsuruo, T.; Furumai, R.; Yoshida, M.; Horinouchi, S.; Hayashi, H. “Cyclic Hydroxamic-acid-containing Peptide 31, a Potent Synthetic Histone Deacetylase Inhibitor with Antitumor Activity” Cancer Res. 2001, 61, 4459-4466.
(69) Krajewski, W. A. “Chromatin structural transitions in Drosophila embryo cell-free extract result in a high conformational flexibility of nucleosomal DNA” FEBS Lett. 1999, 452, 215 -218.
(70) Krajewski, W. A. “Histone Hyperacetylation facilitates chromatin remodelling in a Drosophila embry cell-free system” Mol. Gen. Genet. 2000, 263, 38-47.
(71) Krajewski, W. A. “Histone Acetylation Status and DNA Sequence Modulate ATP-dependent Nucleosome Repositioning”, J. Biol. Chem. 2002, 277, 14509-14513.
(72) Kress, S.; Greenlee, W. F. “Cell-specific regulation of human CYP1A1 and CYP1B1 genes” Cancer Res. 1997, 57, 1264-1269.
(73) Kulys, J.; Deussen, H.-J.; Krikstopaitis, K.; Loick, R.; Schneider, P.; Ziemys, A. “N-Arylhydroxamic Acids as Novel Oxidoreductase Substrates” Eur. J. Org. Chem. 2001, 3475-3484.
(74) Kung, A. L.; Wang, S.; Kico, J. M.; Kaelin, W. G.; Livingston, D. M. “Suppression of tumor growth through disruption of hypoxia-induced transcription”, Nat. Med. 2000, 1335-1340.
(75) Kuriyama, W.; Kitahara, T. “Synthesis of Apicidin” Heterocycles 2001, 55, 1-4.
(76) Kwon, H. J.; Owa, T.; Hassig, C. A.; Shimada, J.; Schreiber, S. L., “Depudecin induces morphological reversion of transformed fibroblasts via the inhibition of histone deacetylase” Proc. Natl. Acad. Sci. USA 1998, 95, 3356.
(77) Kwon, H. J.; Kim, M. S.; Kim, M. J.; Nakjima, H.; Kim, K. W-., “Histone Deacetylase Inhibitor FK228 Inhibits TumorAngiogenesis”, Int.J. Cancer 2002, 97, 290-296.
(78) Lal, A.; Peters, H.; St. Croix, B.; Haroon, Z. A.; Dewhirst, M. W.; Strausberg, R. L.; Kaanders, J. H. A. M.; van der Kogel, A. J.; Riggins, G.J. “Transcriptional response to hypoxia in human tumors” J. Natl. Cancer Inst. 2001, 93, 1337-1343.
(79) Lavoie, R.; Bouchain, G.; Frechette, S.; Woo, S.- Hyung; Khalil, E. A.; Leit, S.; Fournel, M.; Yan, P. T.; Trachy- Bourget, M.-C.; Beaulieu, C.; Li, Z.; Besterman, J.; Delorme, D. “Design and Synthesis of a Novel Class of Histone Deacetylase Inhibitors” Bioorg. Med. Chem. Lett. 2001, 11, 2847-2850.
(80) Li, K. W.; Wu, J.; Xing, W.; Simon, J. A. “Total Synthesis of the Antitumor Depsipeptide FR-901,228”, J. Am. Chem. Soc. 1996, 118, 7237-7238.
(81) Licht, J. D. “Targeting aberrant transcriptional repression in leukemia: a therapeutic reality?”, J. Clin. Invest. 2001, 108, 1277-1278.
(82) Mabjeesh, N. J.; Post, D. E.; Willard, M. T.; Kaur, B.; Van Meir, E. G.; Simons, J. W.; Zhong, H., “Geldanamycin Induces Degradation of Hypoxia-inducible Factor 1α Protein via the Proteasome Pathway in Prostate Cancer Cells”, Cancer Res. 2002, 62, 2478-2482.
(83) Mai, A.; Massa, S.; Ragno, R.; Esposito, M.; Sbardella, G.; Nocca, G.; Scatena, R.; Jesacher, F.; Loidl, P.; Brosch, G. “Binding Mode Analysis of 3-(4-Benzoyl-1-methyl-1H-2-pyrrolyl)-N-hydroxy-2-propenamide: A New Synthetic Histone Deacetylase Inhibitor Inducing Histone Hyperacetylation, Growth Inhibition, and Terminal Cell Differentiation” J. Med. Chem. 2002, 45, 1778-1784.
(84) Malpass, J. R.; Smith, C. “A Simple Approach to Homotropanes and Homotrop-7-enes”, Tet. Lett. 1992, 33, 273-276.
(85) Mamai, A.; Zhang, R.; Natarajann, A.; Madalengoitia, J. S. “Poly-L-proline Type II Peptide Mimetics Based on the 3-Azabicyclo[3.1.0]hexane System” J. Org. Chem. 2001, 66, 455-460.
(86) Marks, P. A.; Richon, V. M.; Breslow, R.; Rifkind, R. A. “Histone Deacetylase Inhibitors as New Cancer Drugs”, Curr. Op. Oncol. 2001, 13, 477-483.
(87) Marmorstein, R. “Structure of Histone Deacetylases: Insights into Substrate Recognition and Catalysis”, Structure 2001, 9, 1127-1133.
(88) Michaelides, M. R.; Dellaria, J. F.; Gong, J.; Holms, J. H.; Bouska, J. J.; Stacey, J.; Wada, C. K.; Heyman, H. R.; Curtin, M. L.; Guo, Y.; Goodfellow, C. L.; Elmore, I. B.; Albert, D. H.; Magoc, T. J.; Marcotte, P. A.; Morgan, D. W.; Davidsen, S. K. “Biaryl Ether Retrohydroxamates as Potent, Long-Lived, Orally Bioa vailable MMP Inhibitors” Bioorg. Med. Chem. Lett. 2001, 11, 1553-1556.
(89) Miller, S. A.; Griffiths, S. L.; Seebach, D. “C-Alkylation of Sarcosine Residues in Cyclic Tetrapeptides via Lithium Enolates” Helv. Chim. Act. 1993, 76, 563-595.
(90) Min, J.; Landry, J.; Sternglanz, R.; Xu, R.-M. “Crystal Structure of a SIR2 Homolog-NAD Complex” Cell, 2001, 705, 269-279.
(91)
(92) Minucci, S.; Nervi, C.; LoCoco, F.; Pelicci, P. G. “Histone Deacetylases: A common molecular target for differentiation treatment of acute myeloid leukemias?”, Oncogene, 2001, 20, 3110-3115.
(93) Missirlis, F.; Phillips, J. P.; Jackie, H. “Cooperative action of antioxidant defense systems in Drosophila” Curr. Biol. 2001, 11, 1272-1277.
(94) Mohammed, J.; Stratford, I. J. “Bioreductive drugs: selectivity towards hypoxic tissue” Expert Opin. Ther. Pat. 1999, 9, 1371-1380.
(95) Moos, P. J.; Edes, K.; Cassidy, P.; Massuda, E.; Fitzpatrick, F. A. “Electrophilic Prostaglandins and Lipid Aldehydes Repress Redox-sensitive Transcription Factors p53 and Hypoxia-inducible Factor by Impairing the Selenoprotein Thioredoxin” J. Biol. Chem. 2003, 278, 745-750.
(96) Murray, P. J.; Kranz, M.; Ladlow, M.; Taylor, S.; Berst, F.; Holmes, A. B.; Keavey, K. N.; Jaxa-Chamiec, A.; Seale, P. W.; Stead, P.; Upton, R. J.; Croft, S. L.; Clegg, W.; Elsegood, M. R. J. “The Synthesis of Cyclic Tetrapeptoid Analogues of the Antiprotozoal Natural Product Apicidin” Bioorg. Med. Chem. Lett. 2001, 11, 773-776.
(97) Naito, S.; Yokomizo, A.; Koga, H. “Mechanisms of Drug Resistance in Chemotherapy for Urogenital Carcinoma”, International Journal of Urology 1999, 6, 427-439.
(98) Ner, S. S.; Blank, T.; Perez-Paralle, M. L.; Grigliatti, T. A.; Becker, P. B.; Travers, A. A. “HMG-D and Histone H1 Interplay during Chromatin Assembly and Early Embryogenesis” J. Biol. Chem. 2001, 276, 37569-37576.
(99) Nicolaou, K. C.; Hwang, C.-K.; Marron, B. E.; DeFrees, S. A.; Couladouros, E. A.; Abe, Y.; Carroll, P. J.; Snyder, J. P. “Bridging of Macrodithiono-lactones to Bicyclic Systems. Synthesis and Modeling of Oxapolycyclic Frameworks” J. Am. Chem. Soc. 1990, 772, 3040-3055.
(100) O'Reilly, M. S.; Boehm, T.; Shing, Y.; Fukai, N.; Vasios, G.; Lane, W. S.; Flynn, E.; Birkhead, J .R.; Olsen, B. R.; Folkman, J. “Endostatin: An Endogenous Inhibitor of Angiogenesis and Tumor Growth” Cell 1997, 88, 277-285.
(101) Okajima, T.; Irvine, K. D. “Regulation of notch signaling by O-linked fucose” Cell 2002, 111, 893-904.
(102) Ozawa, Y.; Towatari, M.; Tsuzuki, S.; Hayakawa, F.; Maeda, T.; Miyata, Y.; Tanimoto, M.; Saito, H. “Histone Deacetylase 3 Associates with and represses the transcription factor GATA-2′, Blood, 2001, 98, 2116-2123.
(103) Paddison, P. J.; Hannon, G. J. “RNA Interference: the new somatic cell genetics?” Cancer Cell 2002, 2, 17-23.
(104) Pan, D. A.; Hardie, D. G. “A homologue of AMP-activated protein kinase in Drosophila melanogaster is sensitive to AMP and is activated by ATP depletion” Biochem. J. 2002, 367, 179-186.
(105) Pastuszak, J.; Gardner, J. H.; Singh, J.; Rich, D. H. “Improved Synthesis of [4-Alanine]chlamydocin: Cyclization studies of Tetrapeptides Containing Five α-Substituents” J. Org. Chem. 1982, 47, 2982-2987.
(106) Patra, S. K.; Patra, A.; Dahiya, R. “Histone Deacetylase and DNA Methyltransferase in Human Prostate Cancer”, Biochem. Biophys. Res. Comm. 2001, 287, 705-713.
(107) Patterson, L. H.; Raleigh, S. M. “Reductive Metabolism: its application in prodrug activation” Biomed. Health Res. 1998, 25, 72-79.
(108) Paz, M.; Hopkins, P. B. “DNA-DNA Interstrand Crosslinking by FR-66979: Intermediates in the Activation Cascade”, J. Am. Chem. Soc. 1997, 119, 5999-6005.
(109) Pazin, M. J.; Kadonaga, J. T., “What's Up and Down with Histone Deacetylation and Transcription?”, Cell 1997, 89, 325.
(110) Pellicena-Palle, A.; Stitzinger, S. M.; Salz, H. K. “The function of the Drosophila thioredoxin homologue encoded by the deadhead gene is redox-dependent and blocks the initiation of development but not DNA synthesis”, Mechanisms of Development 1997, 62, 61-65.
(111) Peterson, C. L. “Chromatin: Mysteries Solved?” Biochem. Cell. Biol. 2001, 79, 219-225.
(112) Pilch, H.; Schlenger, K.; Steiner, E.; Brockerhoff, P.; Knapstein, P.; Vaupel, P. “Hypoxia-stimulated expression of angiogenic growth factors in cervical cancer cells and cervical cancer-derived fibroblasts” Int. J. Gyn. Cancer 2001, 11, 137-142.
(113) Pile, L. A.; Wassarman, D. A. “Chromosomal localization links the SIN3-RPD3 complex to the regulation of chromatin condensation, histone acetylation and gene expression” EMBOJ. 2000, 19, 6131-6140.
(114) Prives, C.; Manley, J. L. “Why is p53 Acetylated”, Cell 2001, 107, 815-818.
(115) Rajski, S. R.; Williams, R. M. “DNA Cross-linking Agents as Antitumor Drugs” Chem. Rev. 1998, 98, 2723-2795.
(116) Ratcliffe, P. J.; Pugh, C. W.; Maxwell, P. H. “Targeting tumors through the HIF Systenm”, Nat. Med. 2000, 6, 1315-1316.
(117) Razin, A. “CpG methylation, chromatin structure and gene silencing-a three way connection”, EMBO J. 1998, 17, 4905-4908.
(118) Reiher, F. K.; Ivanovich, M.; Huang, H. H.; Smith, N. D.; Bouck, N. P.; Campbell, S. C. “The role of hypoxia and p53 in the regulation of angiogenesis in bladder cance! J. Urology 2001, 165, 2075-2081.
(119) Rikhy, R.; Kumar, V.; Mittal, R.; Krishnan, “Endophilin Is Critically Required for Synapse Formation and Function in Drosophila melanogaster” J. Neurosci. 2001, 22, 7478-7484.
(120) Roth, S. Y.; Allis, C. D., “Histone Acetylation and Chromatin Assembly: A Single Escort, Multiple Dances?” Cell, 1996, 87, 5.
(121) Rudin, C. M.; Yang, Z.; Schumaker, L. M.; VanderWeele, D. J.; Newkirk, K.; Egorin, M. J.; Zuhowski, E. G.; Cullen, K. J. “Inhibition of Glutathione Synthesis Reverses Bcl-2-mediated Cisplatin Resistance” Cancer Res. 2003, 63, 312-318.
(122) Saramaki, O. R.; Savinainen, K. J.; Nupponen, N. N.; Bratt, O.; Visakorpi, T. “Amplification of hypoxia-inducible factor 1α gene in prostate cancer” Cancer Genetics and Cytogenetics 2001, 128, 31-34.
(123) Schreiber, S. L.; Claus, R. E.; Reagan, J. “Ozonlytic Cleavage of Cycloalkenes to Terminally Differentiated Products”, Tet. Lett. 1982, 23, 3867-3870.
(124) Semenza, G. L. “HIF-1: Using two hands to flip the angiogenic switch”, Cancer and Metastasis Reviews 2000,19, 59-65.
(125) Semenza, G. L. “HIF-1 and mechanisms of hypoxia sensing” Curr. Op. Cell Bio. 2001, 13, 167-171.
(126) Shao, Li-en, Diccianni, M. B.; Tanaka, T.; Gribi, R.; Yu, A. L.; Pullen, J. D.; Camitta, B. M.; Yu, J. “Thioredoxin Expression in Primary T-Cell Acute Lymphoblastic Leukemia and Its Therapeutic Implication” Cancer Res. 2001, 67, 7333-7338.
(127) Shigeyuki, K.; Kazumi, S.; Kiyoshi, T. “Reductase activity of aldehyde oxidase toward the carcinogen N-hydroxy-2-acetylaminofluorene and the related hydroxamic acids” Biochem. and Mol. Biol. Int. 1994, 34,1197-1203.
(128) Shimo, T.; Kubota, S.; Kondo, S.; Nakanishi, T.; Sasaki, A.; Mese, H.; Matsumura, T.; Takigawa, M. “Connective tissue growth factor as a major angiogenic agent that is induced by hypoxia in a human breast cancer cell line” Cancer Letters 2001, 174, 57-64.
(129) Shireman, B. T.; Miller, M. J.; Jonas, M.; Wiest, O. “Conformational Study and Enantioselective, Regiospecific Syntheses of Novel Aminoxy trans-Proline Analogues Derived from an Acyinitroso Diels-Alder Cycloaddition” J. Org. Chem. 2001, 66, 6046-6056.
(130) Shireman, B. T.; Miller, M. J. “Synthesis of Enantiomerically and Diastereomerically Pure 2(S)-Amino-6(R)-hydroxy-1,7-heptanedoic Acid Dimethyl Ester Hydrochloride from Cycloheptadiene” J. Org. Chem. 2001, 66, 4809-4813.
(131) Shute, R. E.; Rich, D. H. “A Novel Double Deptrotection Peptide Cyclization Procedure and Its Application to the Synthesis of Analogues of the Cyclic Tetrapeptide HC-Toxin” Tet. Lett. 1987, 28, 3419-3422.
(132) Shute, R. E.; Dunlap, B.; Rich, D. H. “Analogues of the Cytostatic and Antimitogenic Agents Chlamydocin and HC-Toxin: Synthesis and Biological Activity of Chloromethyl Ketone and Diazomethyl Ketone Functionalized Cyclic Tetrapeptides” J. Med. Chem. 1987, 30, 71-78.
(133) Shute, R. E.; Kawai, M.; Rich, D. H. “Conformationally Constrained Biologically Active Peptides: Tentative Identification of the Antimitogenic Conformer of the Naturally Occurring Cyclic Tetrapeptides” Tetrahedron, 1988, 44, 685-695.
(134) Smith, W. L.; Edlind, T. D. “Histone Deacetylase Inhibitors Enhance Candida albicans Sensitivity to Azoles and Related Antifungals: Correlation with Reductions in CDR and ERG Upregulation”, Antimicrobial Agents & Chemotherapy 2002, 46, 3532-3539.
(135) Song, Y.; Clizbe, L.; Bhakta, C.; Teng, W.; Wong, P.; Huang, B.; Tran, K.; Sinha, U.; Park, G.; Reed, A.; Scarborough, R. M.; Zhu, B.-Y. “Design and Synthesis of Factor Xa Inhibitors and Their Pro-drugs” Bioorg. Med. Chem. Lett. 2003, 73, 297-300.
(136) Spink, D. C.; Spink, B. C.; Cao, J. Q.; DePasquale, J. A.; Pentecost, B. T.; Fasco, M. J.; Li, Y.; Steffan, J. S.; Bodai, L.; Pallos, J.; Poelman, M.; McCampbell, A.; Apostol, B. L.; Kazantsev, A.; Schmidt, E.; Zhu, Y.-A.; Greenwald, M.; Kurokawa, R.; Housman, D. E.; Jackson, G. R.; Marsh, J. L.; Thompson, L. M. “Histone Deacetylase Inhibitors Arrest Polyglutamine-dependent Neurodegeneration in Drosophila” Nature 2001, 473, 739-743.
(137) Starai, V. J.; Celic, I.; Cole, R. N.; Boeke, J. D.; Escalaente-Semerena, J. C. “Sir2-Dependent Activation of Acetyl-CoA Synthetase by Deacetylation of Active Site Lysine” Science 2002, 298, 2390-2392;
(138) Sternson, S. M.; Wong, J. C.; Grozinger, C. M.; Schreiber, S. L. “Synthesis of 7200 Small Molecules Based on a Substructural Analysis of the Histone Deacetylase Inhibitors Trichostatin and Trapoxin”, Org. Lett. 2001, 3, 4239-4242.
(139) Su, G. H.; Sohn, T. A.; Ryu, B.; Kern, S. E. “A Novel Histone Deacetylase Inhibitor Identified by High-Throughput Transcriptional Screening of a Compound Library” Cancer Res. 2000, 60, 3137-3142.
(140) Sugihara, K.; Kitamura, S.; Tatsumi, K. “Evidence for Reduction of Hydroxamic Acids to the Corresponding Amides by Liver Aldehyde Oxidase” Chem. Pharm. Bull. 1983, 31, 3366-3369.
(141) Sugihara, K.; Kitamura, S.; Ohta, S.; Tatsumi, K. “Reduction of hydroxamic acids to the corresponding amides catalyzed by rabbit blood”, Xenobiotica, 2000, 30, 457-467.
(142) Sun, X. Y.; Kanwar, J. R.; Leung, E.; Lehnert, K.; Wang, D. M.; Krissansen, G. W. “Angiostatin enhances B7.1-mediated cancer immunotherapy independently of effects on vascular endothelial growth factor expression” Cancer Gene Therapy 2001, 8, 719-727.
(143) Surman, M. D.; Miller, M. J. “Regio- and Stereochemically Controlled Formation of Hydroxamic Acid Containing anti- or syn-1,4-Cycloalkenols from Acyinitroso-Derived Diels-Alder Adducts” J. Org. Chem. 2001, 66, 2466-2469.
(144) Sutter, T. R. “Differential Expression of CYP1A1 and CYP1B1 in human breast epithelial cells and breast tumor cells”, Carcinogenesis 1998, 19, 291-298.
(145) Taunton, J.; Collins, J. L.; Schreiber, S. L., “Synthesis of Natural and Modified Trapoxins, Useful Reagents for Exploring Histone Deacetylase Function”, J. Am. Chem. Soc. 1996, 118, 10412.
(146) Tong, J. K. “Dissecting Histone Deacetylase Function”, Chem. Biol. 2002, 9, 668-670.
(147) Tsai, C.-Y.; Huang, X.; Wong, C.-H. “Design and synthesis of cyclic sialyl Lewis X mimetics: a remarkable enhancement of inhibition by pre-organizing all essential functional groups”, Tet. Lett. 2000, 41, 9499-9503.
(148) Tseng, A.-S. K.; Hariharan, I. K. “An overexpression screen in drosophila for genes that restrict growth or cell-cycle progression in the developing eye” Genetics 2002, 162, 229-243.
(149) Turner, B. M. “Cellular Memory and the Histone Code”, Cell 2002, 111, 285-291.
(150) Uesato, S.; Kitagawa, M.; Nagaoka, Y.; Maeda, T.; Kuwajima, H.; Yamori, T. “Novel Histone Deacetylase Inhibitors: N-Hydroxycarboxamides Possessing a Terminal Bicyclic Aryl Group” Bioorg. Med. Chem. Lett. 2002, 12, 1347-1349.
(151) Vaziri, H.; Dessain, S. K.; Eaton, E. N.; Imai, S.-I.; Frye, R. A.; Pandita, T. K.; Guarente, L.; Weinberg, R. A. “hSIR2^{SIRT1} Functions as an NAD-Dependent p53 Deacetylasde Cell, 2001, 707, 149-159.
(152) Verdoucq, L.; Vignols, F.; Jacquot, J.-P.; Chartier, Y.; Meyer, Y. “In Vivo Characterization of a Thioredoxin h Target Protein Defines a New Peroxiredin Family” J. Biol. Chem. 1999, 274, 19714-19722.
(153) Vigushin, D. M.; Coombes, R. C. “Histone Deacetylase Inhibitors in Cancer Treatment”, Anti-Cancer Drugs, 2002, 13, 1-13.
(154) Volante, R. P. “A New, Highly Efficient Method for the Conversion of Alcohols to Thiolesters and Thiols” Tet. Lett. 1981, 22, 3119-3122.
(155) Wade, P. A.; Gegonne, A.; Jones, P. L.; Ballestar, E.; Aubry, F.; Wolffe, A. P. “Mi-2 complex couples DNA methylation to chromatin remodelling and histone deacetylation”, Nat. Genet. 1999, 23, 62-66.
(156) Wei, Y.; Yi, T.; Huntington, K. M.; Chaudhury, C.; Pei, D. “Identification of a Potent Peptide Deformylase Inhibitor from a Rationally Designed Combinatorial Library”, J. Comb. Chem. 2000, 2, 650-657.
(157) Welsh, S. J.; Williams, R.; Birmingham, A.; Newman, D. J.; Kirkpatrick, D. L.; Powis, G. “The Thioredoxin Redox Inhibitors 1-methylpropyl 2-imidazolyl disulfide and Pleurotin Inhibit Hypoxia-induced Factor 1α and Vascular Endotheliel Growth Factor Formation” Mol. Cancer. Ther. 2003, 2, 235-243.
(158) Williams, R. J. “Trichostatin A, An Inhibitor of Histone Deacetylase, Inhibits Hypoxia-Induced Angiogenesis” Expert Opin. Invest. Drugs 2001, 10, 1571-1573.
(159) Wipf, P.; Miller, C. P. “An investigation of the Mitsunobu reaction in the preparation of peptide oxazolines, thiazolines, and aziridines” Tet. Lett. 1992, 33, 6267-6270.
(160) Wipf, P.; Jeger, P.; Kim, Y. “Thiophilic Ring-Opening and Rearrangement Reactions of Epoxyketone Natural Products”, Bioorg. Med. Chem. Lett. 1998, 8, 351-356.
(161) Wittich, S.; Scherf, H.; Xie, C.; Brosch, G.; Loidi, P.; Gerhauser, C.; Jung, M. “Structure-Activity Relationships on Phenylalanine-Containing Inhibitors of Histone Deacetylase: In Vitro Enzyme Inhibition, Induction of Differentiation, and Inhibition of Proliferation in Friend Leukemic Cells” J. Med. Chem. 2002, 45, 3296-3309.
(162) Woo, S. H.; Frechette, S.; Khalil, E.A .; Bouchain, G.; Vaisburg, A.; Bernstein, N.; Moradei, O.; Leit, S.; Allen, M.; Fournel, M.; Trachy-Bourget, M.-C.; Li, Z.; Besterman, J. M.; Delorme, D. “Structurally Simple Trichostatin A-Like Straight Chain Hydroxamates as Potent Histone Deacetylase Inhibitors” J. Med. Chem. 2002, 45, 2877-2885.
(163) Yoshida, M.; Nomura, S.; Beppu, T., “Effects of Trichostatins on Differentiation of Murine Erythroleukemia Cells”, Cancer Res. 1987, 47, 3688.
(164) Zhang, Y.; Reinberg, D. “Transcriptional regulation by histone methylation: interplay between different covalent modifications of the core histone tails”, Genes & Development 2001, 15, 2343-2360.
(165) Zhu, X. F.; Williams, H. J.; Scott, A. I. “Facile and highly selective 5′-desilylation of multisilylated nucleosides”, J. Chem. Soc. Perkin I 2000, 15, 2305-2306.
(166) Zoller, T.; Ducep, J.-B.; Tahtaoui, C.; Hlbert, M. “Cyclo-release synthesis of cyclic disulfides on solid phase” Tet. Lett. 2000, 47, 9989-9992.

What is claimed is:

1. A composition for inhibiting a histone deacetylase comprising a compound represented by the general formula: 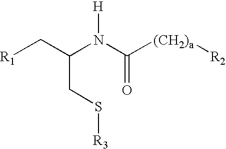
wherein R₁ is —OH, —NH, —NHR or a cap structure selected from the group consisting of unsubstituted and substituted alkyls, alkenyls, alkynyls, cycloalkyls, aryls, heterocyclyls, phthalimides, naphthalimides and polycyclic phenols;
wherein R₂ is a —SH or —SCOCH₃;
wherein R₃ is —H or a structure selected from the group consisting of unsubstituted and substituted alkyls, alkenyls, alkynyls, cycloalkyls, aryls, and heterocyclyls; and
wherein n is an integer from 3 to 7.

2. A composition according to claim 1 wherein R₁ is a tert-butyidiphenylsilyl (TBSPS—O—) group or

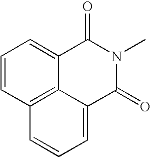

3. A composition according to claim 1 wherein R₁ is a cap structure selected from the group consisting of:

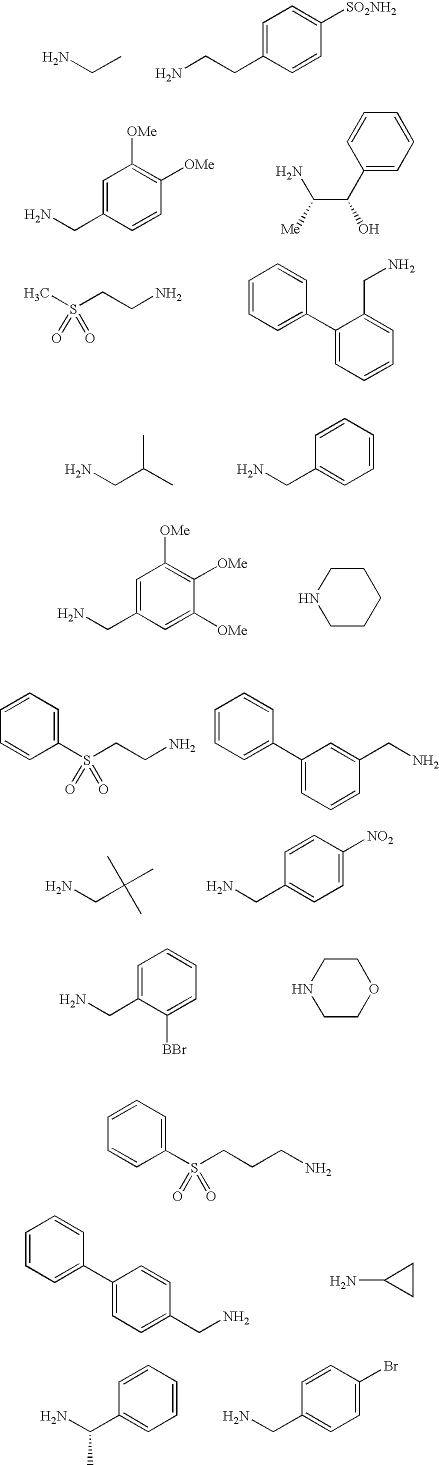

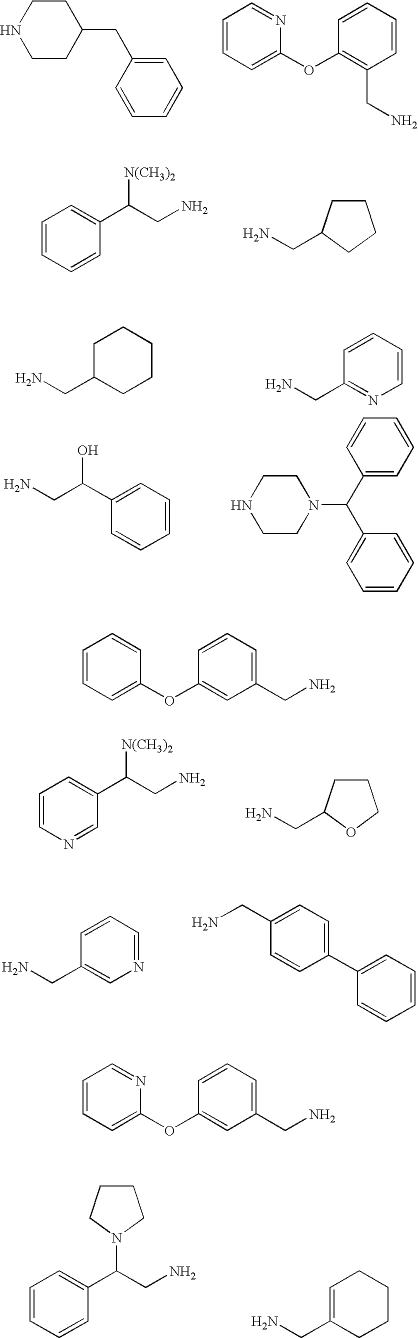

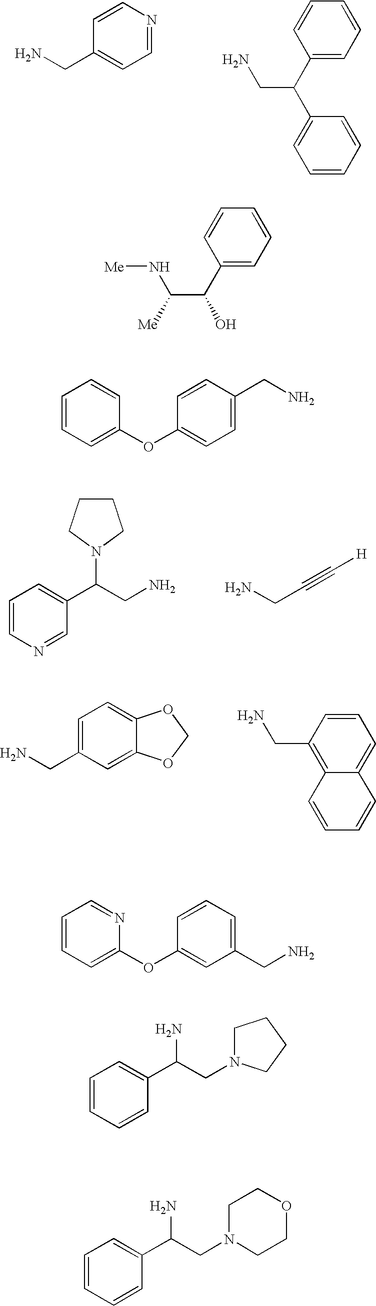

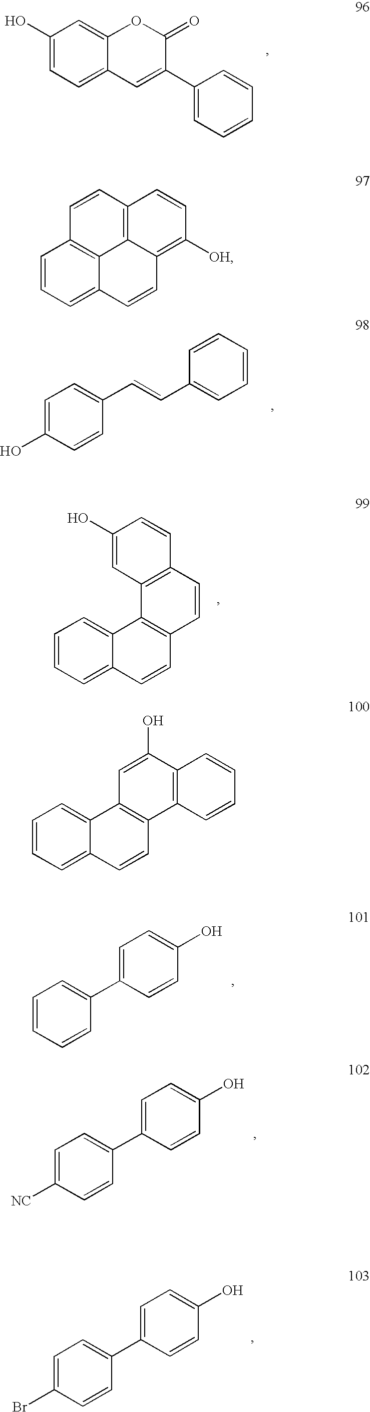

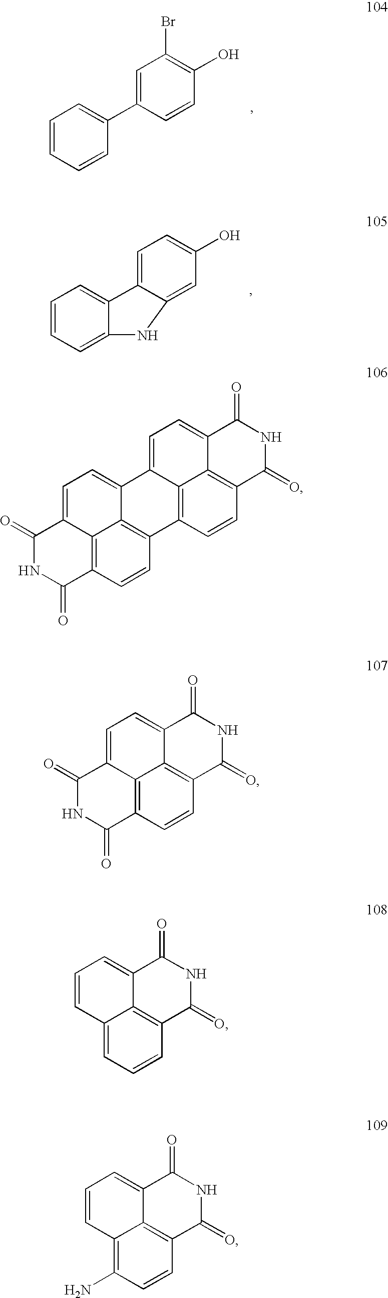

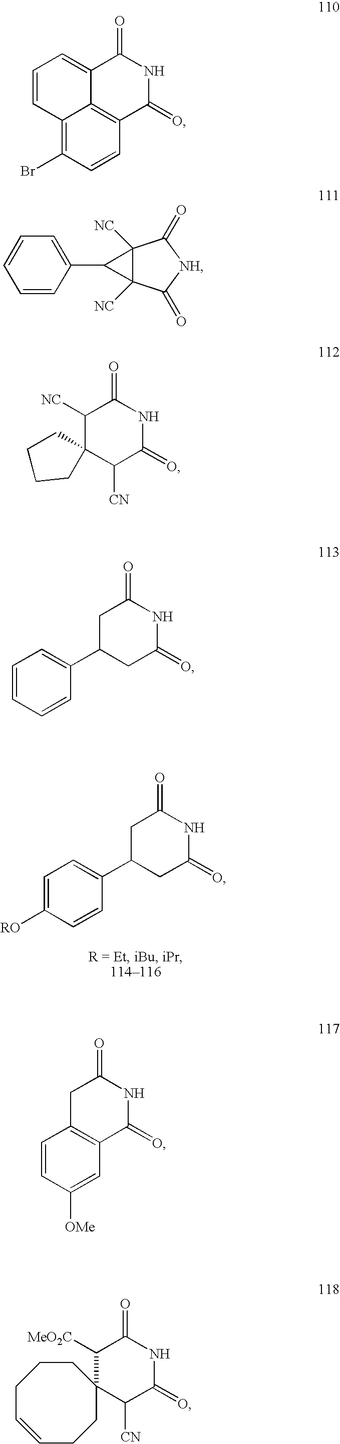

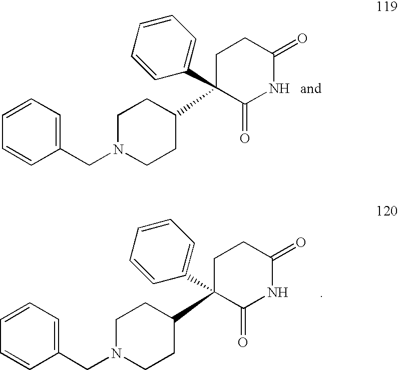

4. A composition according to claim 1 wherein the compound has the formula:

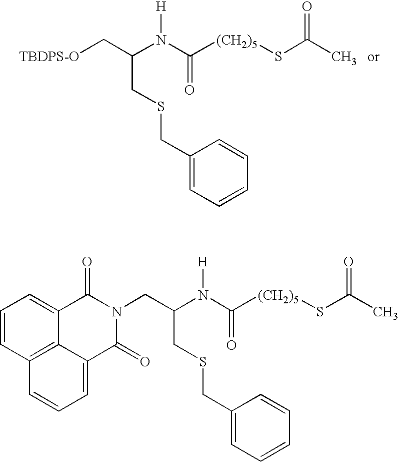

5. A composition according to claim 1 wherein the compound has the formula:

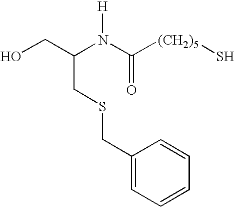

6. A composition according to claim 1 wherein n equals 5.

7. A composition for inhibiting a histone deacetylase comprising a compound represented by the general formula: 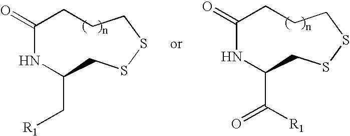
wherein n is an integer from 1 to 7; and
wherein R₁ is —OH, RCONH₂ or a cap structure wherein R is selected from the group consisting of unsubstituted and substituted alkyls, alkenyls, alkynyls, cycloalkyls, aryls, heterocyclyls and the cap structure is selected from the group consisting of unsubstituted and substituted alkyls, alkenyls, alkynyls, cycloalkyls, aryls, heterocyclyls, phthalimides, naphthalimides and polycyclic phenols.

8. A composition according to claim 7 wherein n equals 5.

9. A composition according to claim 6 wherein wherein R₁ is a cap structure selected from the group consisting of:

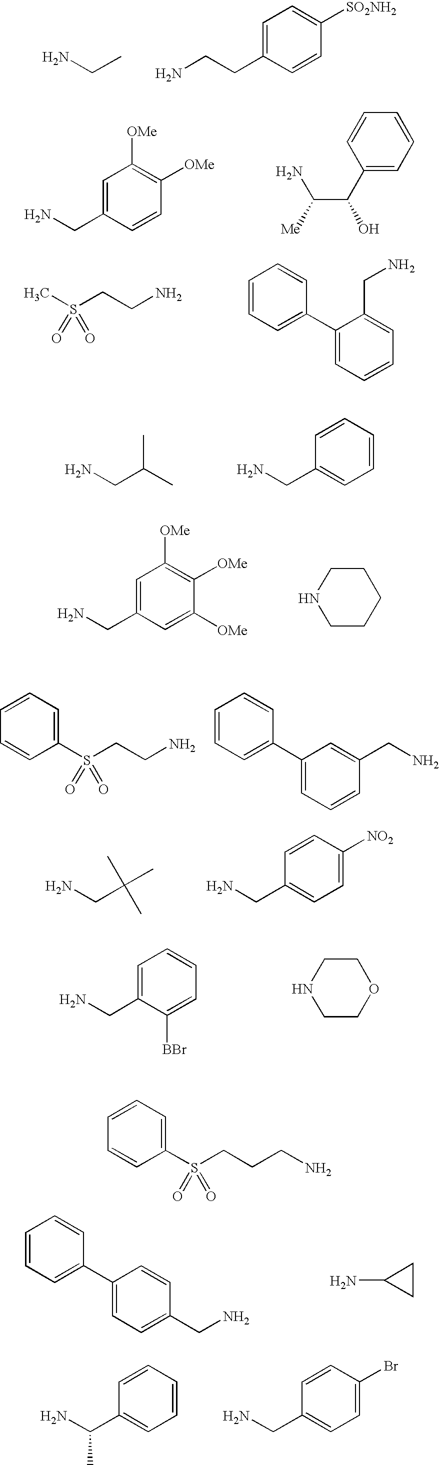

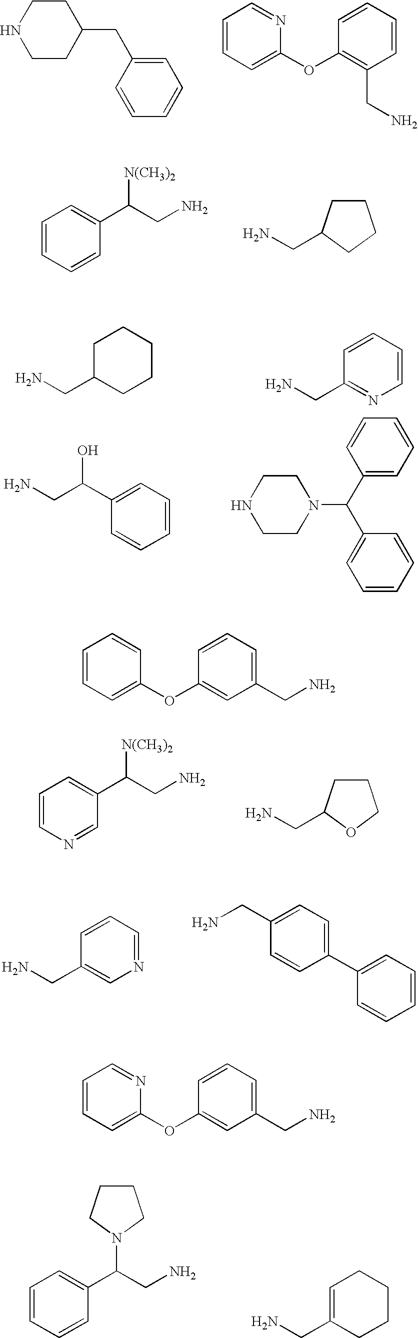

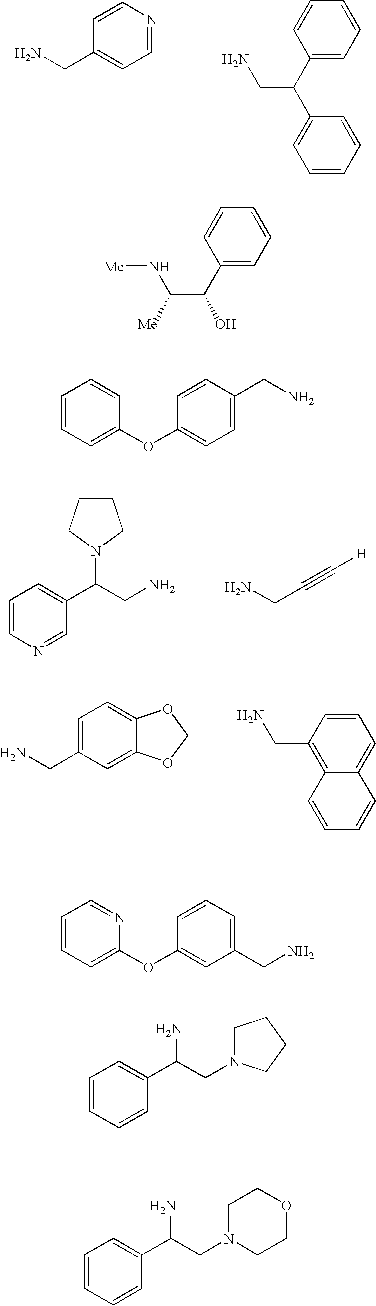

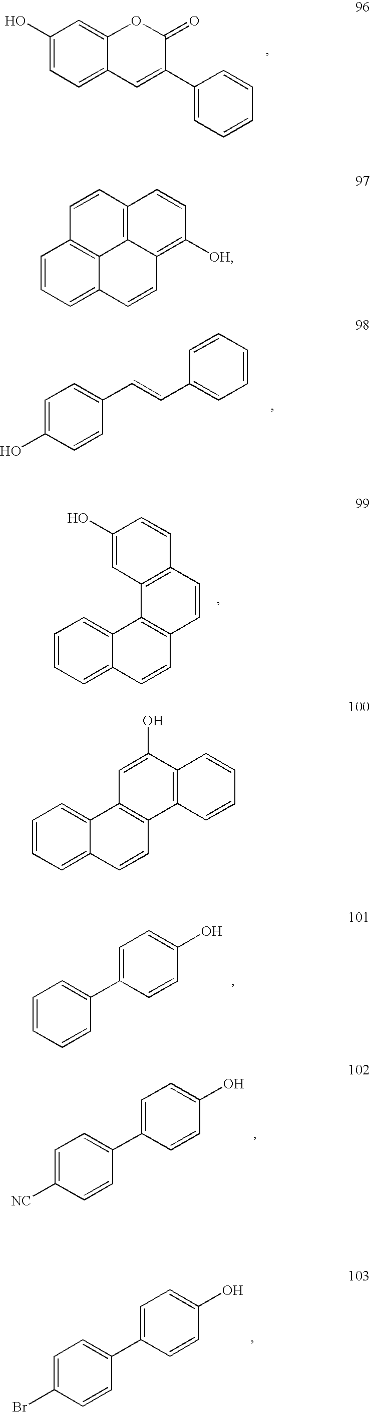

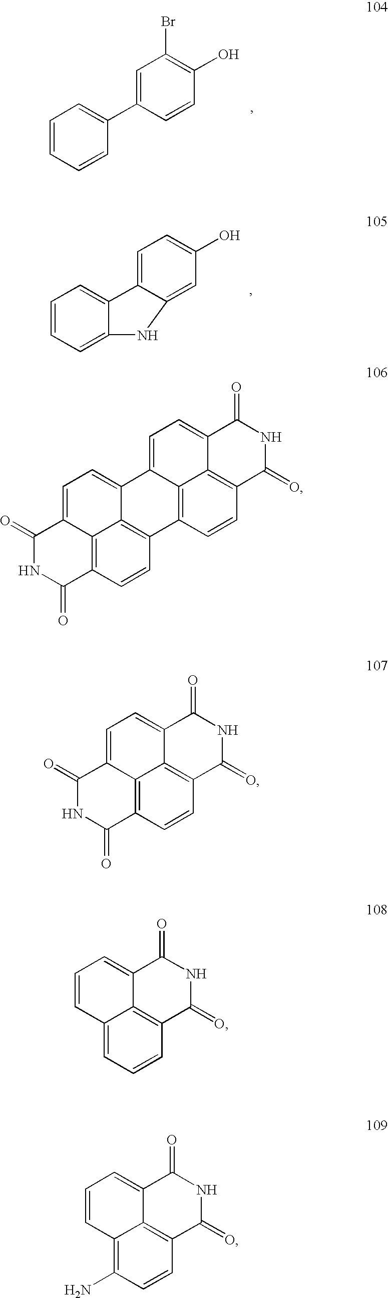

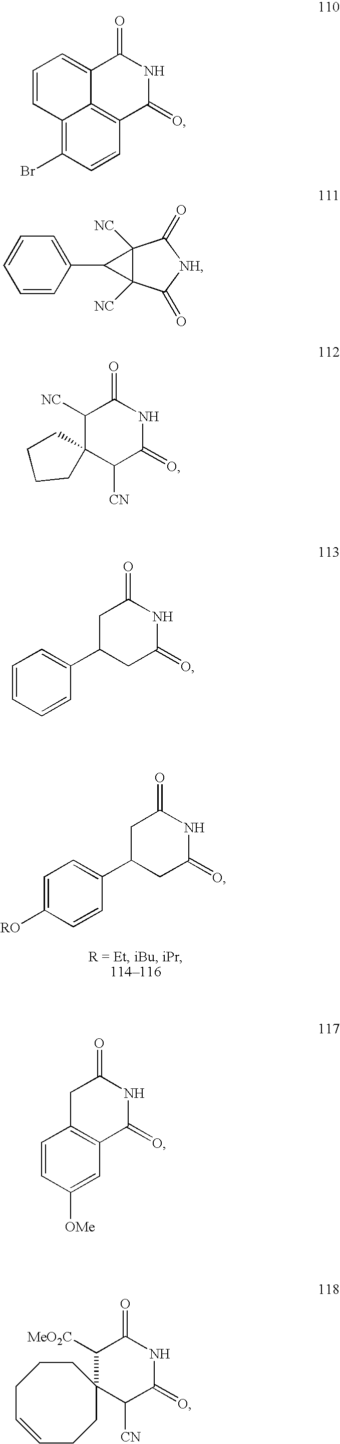

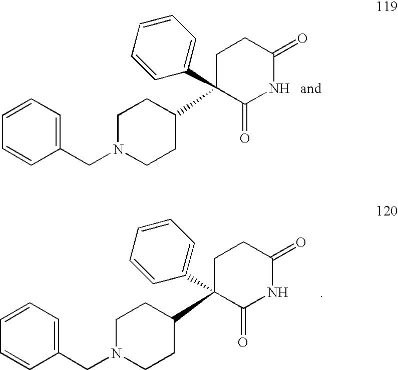

10. A method of synthesizing a cyclized disulfide compound for inhibiting a histone deacetylase, comprising steps of chemically converting:
(a) a lactone to a corresponding TBS silyl ether lactone;
(b) said TBS silyl ether lactone to a corresponding acetylthiol;
(c) said acetylthiol to a corresponding thiol;
(d) said thiol to a corresponding cyclized disulfide compound for inhibiting a histone deacetylase.

11. A method according to claim 10 further comprising the step of coupling said cyclized disulfide compound to a targeting agent.

12. A method according to claim 10 wherein said targeting agent is a monoclonal antibody, N-benzylpolyamine, porphyrin, a polunucleotide encoding a modulating agent, thioredoxin, thioredoxing reductase, or funtional analog thereof.

13. A method according to claim 11 wherein said targeting agent is a capping group selected from the group consisting of:

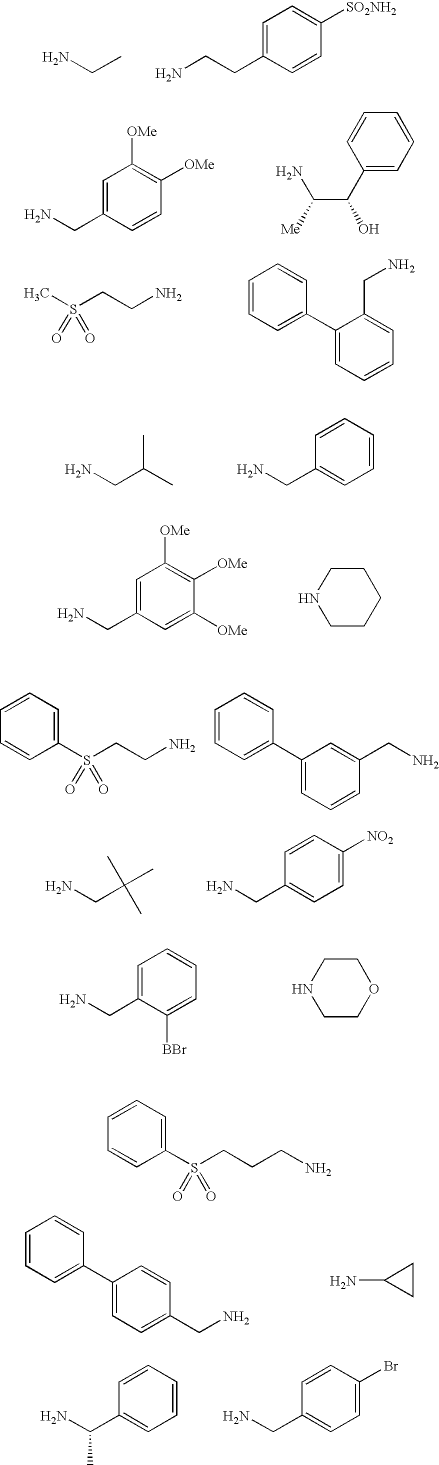

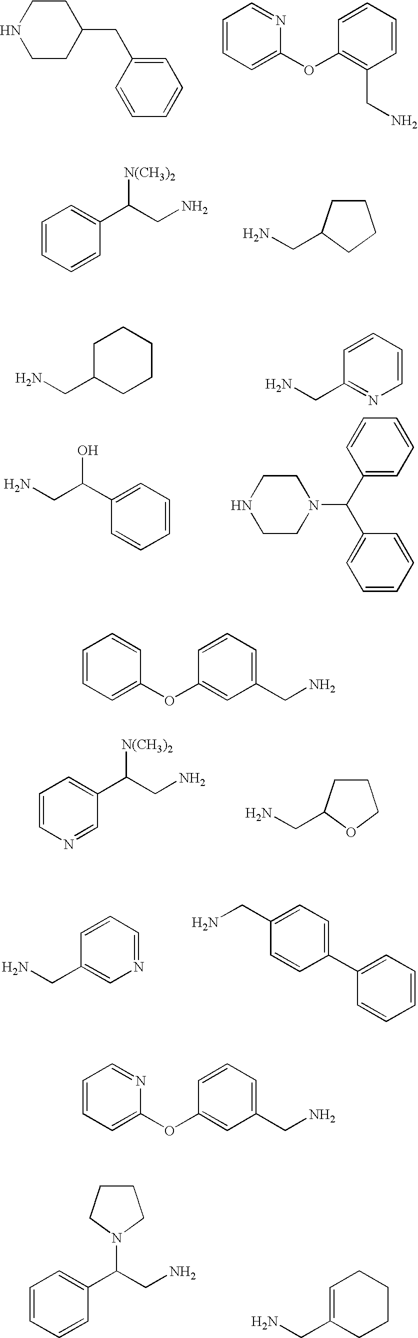

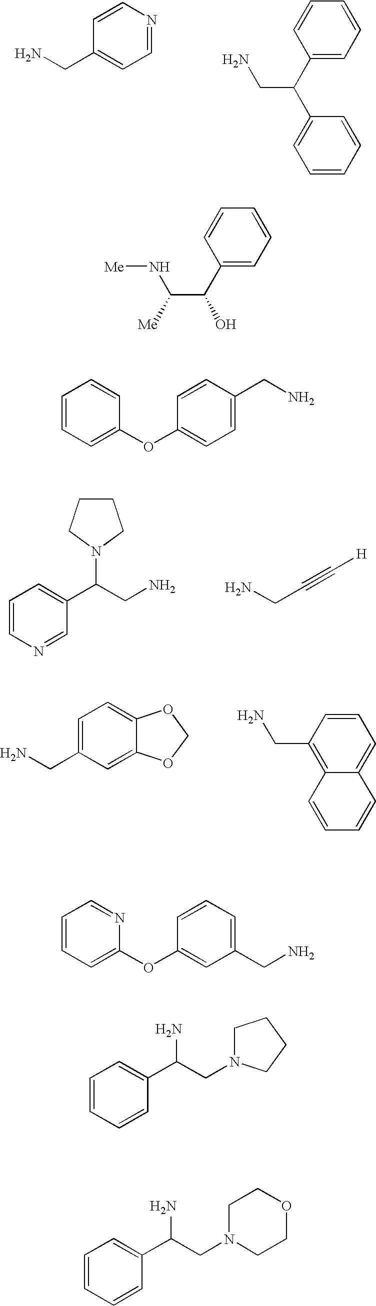

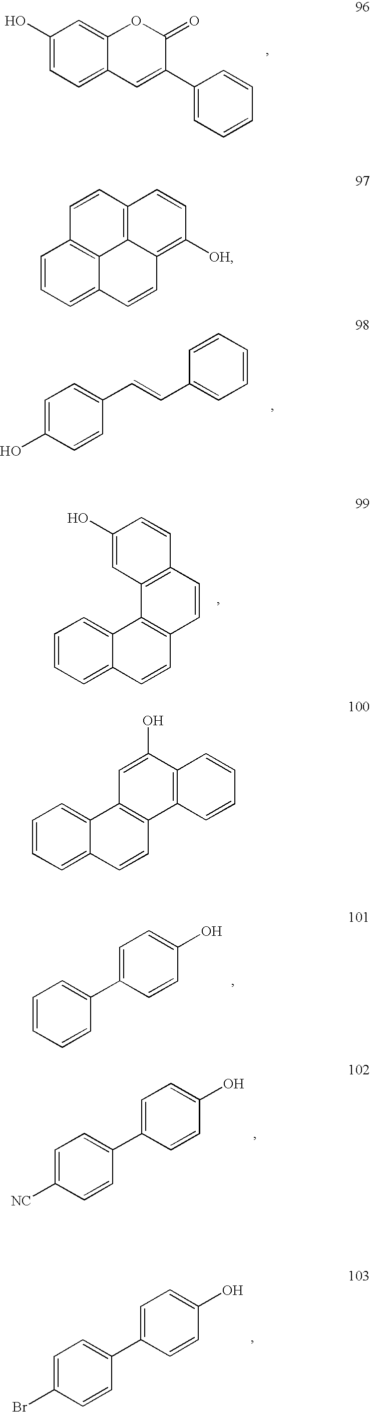

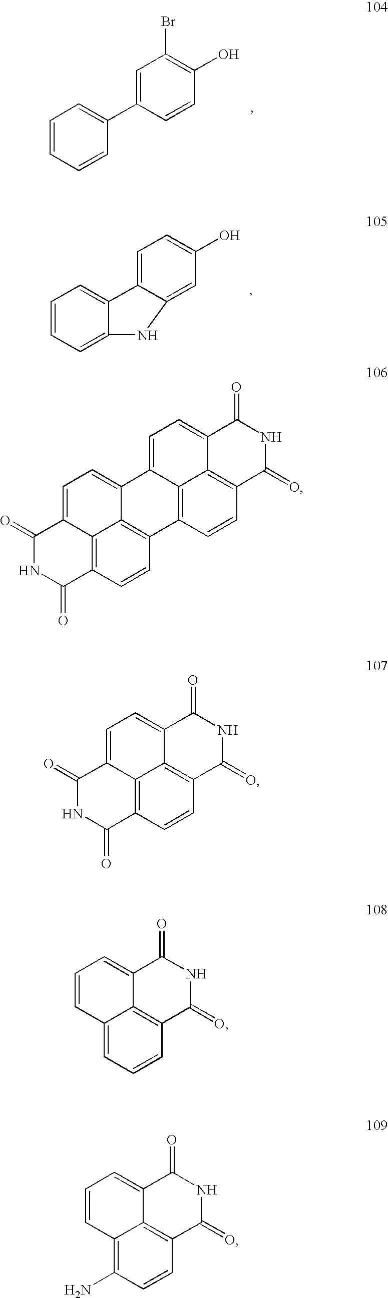

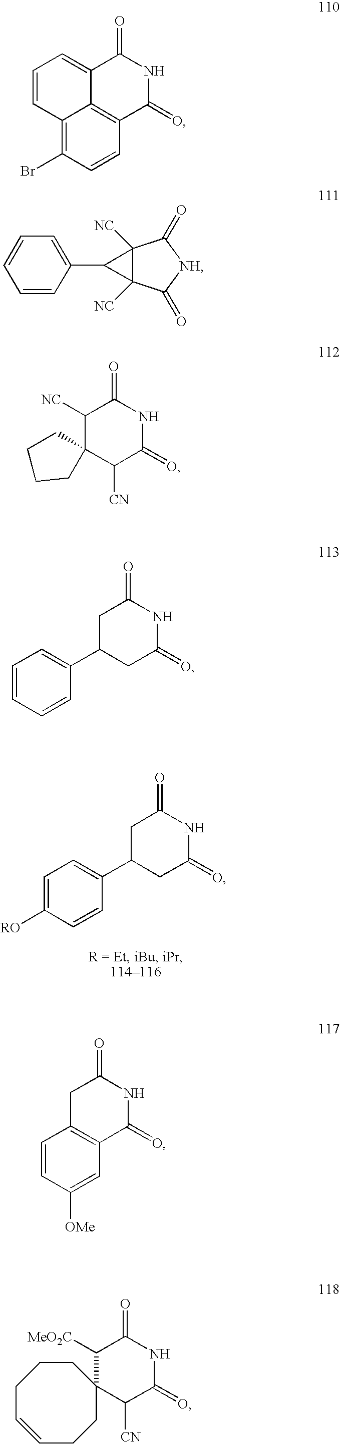

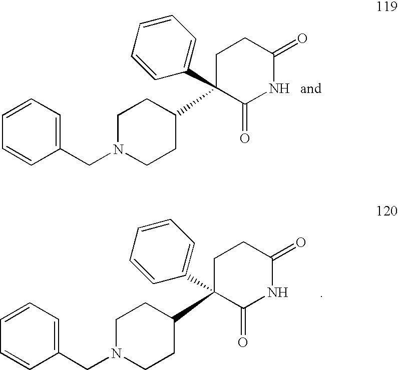

14. A method of synthesizing a cyclized disulphide compound for inhibiting a histone deacetylase, comprising steps of chemically converting: (a) a bromoacid to a corresponding ditritylated ester; (b) the ditritylated ester to a corresponding macrocycle; (c) the macrocycle to a corresponding cyclized disulphide compound via reduction, for inhibiting a histone deacetylase.

15. A method according to claim 14, further comprising the step of coupling said cyclized disulfide compound to a targeting agent.

16. A method according to claim 15, wherein said targeting agent is a monoclonal antibody, N-benzylpolyamine, porphyrin, a polunucleotide encoding a modulating agent, thioredoxin, thioredoxing reductase, or funtional analog thereof.

17. A method according to claim 15, wherein said targeting agent is a capping group selected from the group consisting of:

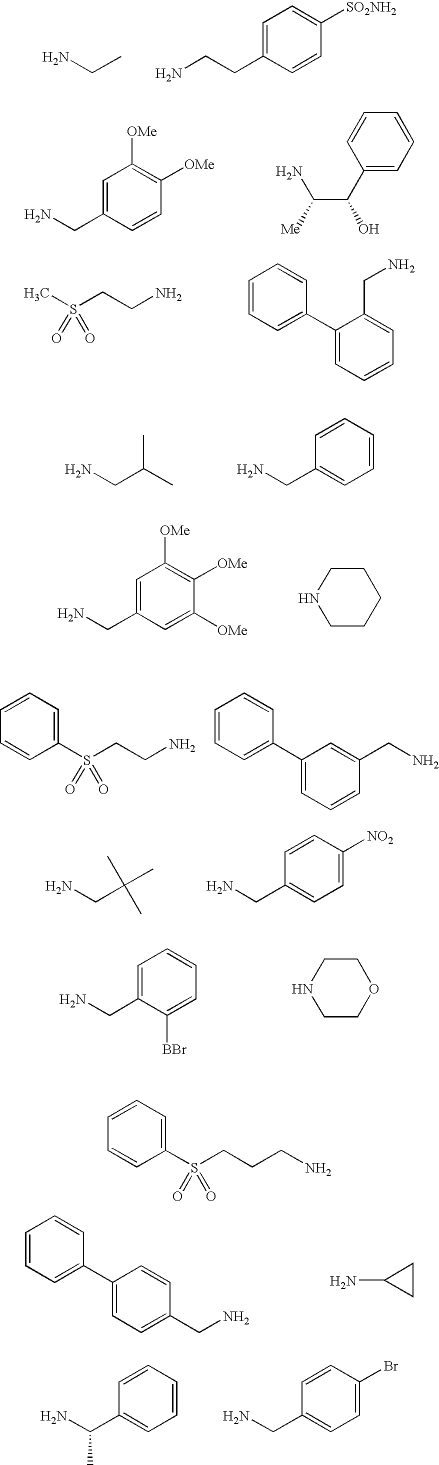

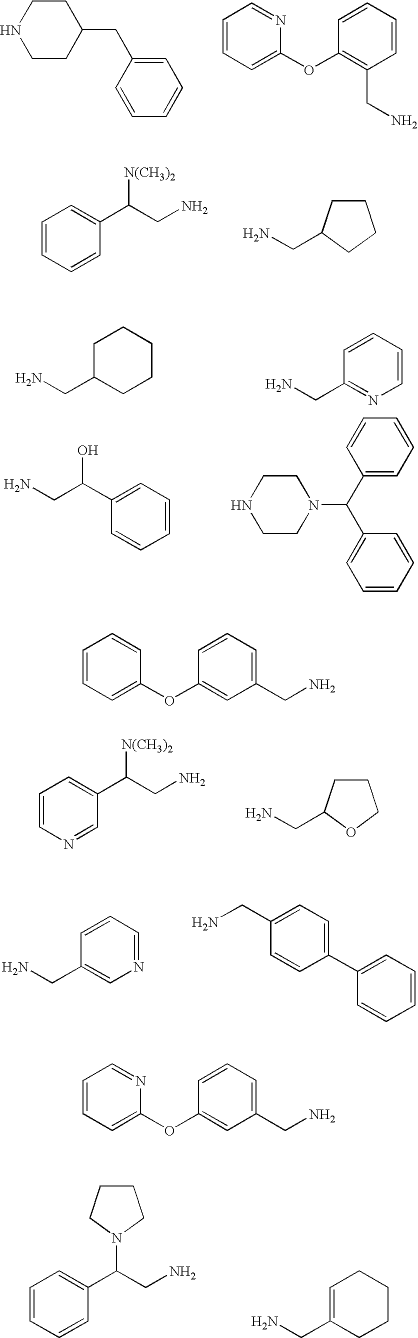

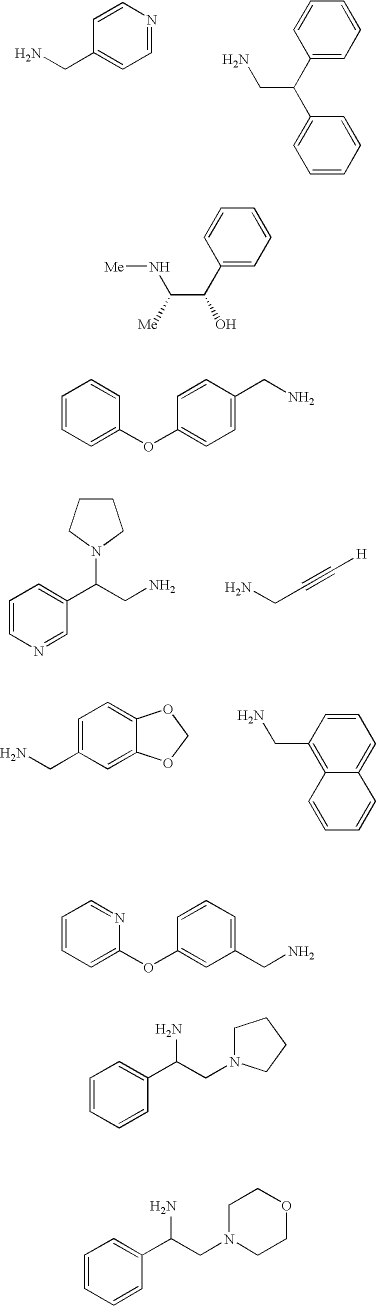

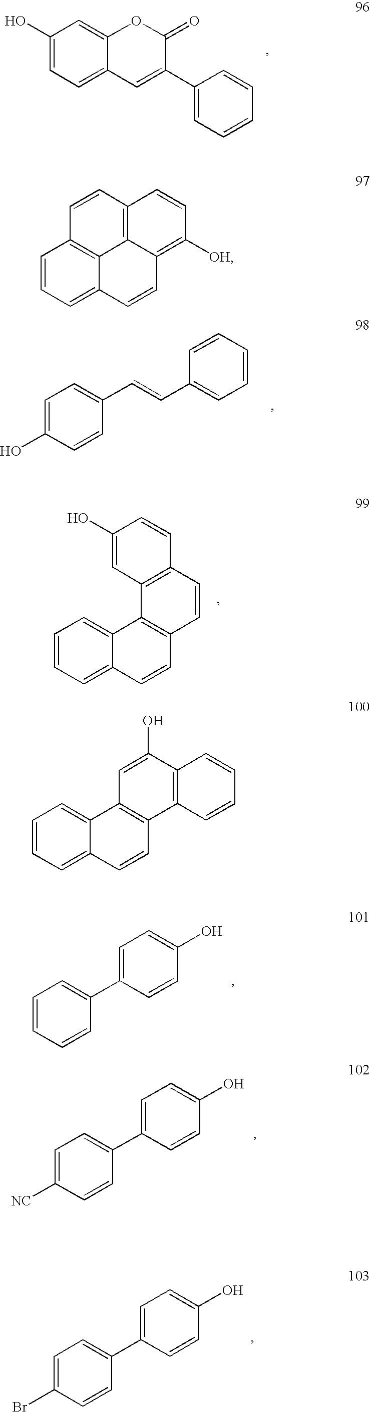

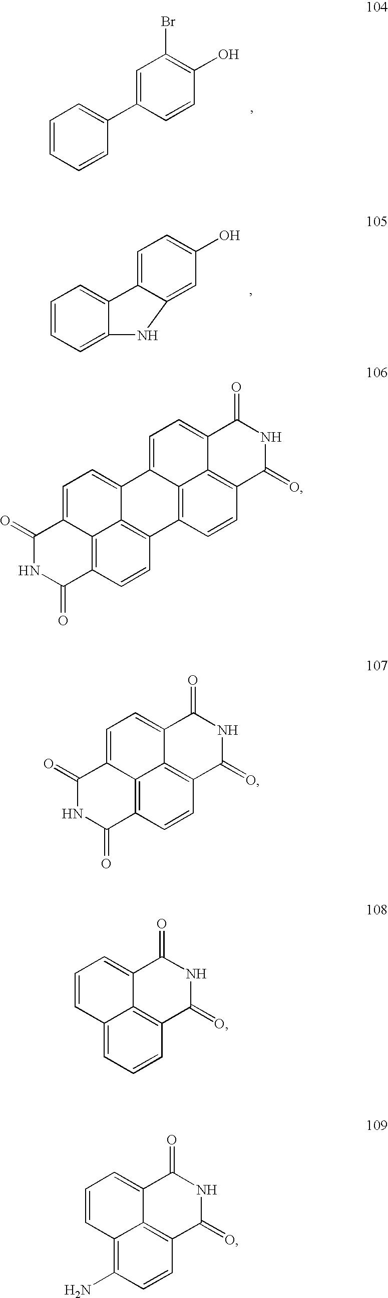

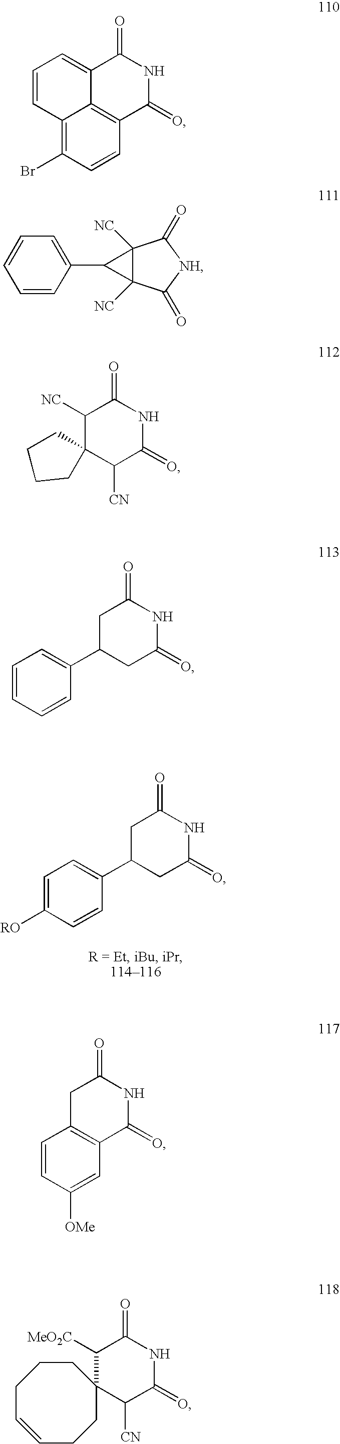

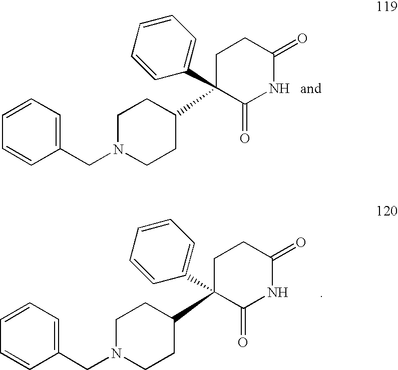

18. A compound produced by the method of any of claims 10 or 14.

19. A pharmaceutical composition for inhibiting a histone deacetylase, comprising a composition according to claim 1 or 7 and a pharmaceutically-acceptable carrier.

20. A method of eliciting a chemopreventive effect for a disease in a patient comprising the step of administering a pharmaceutically effective amount of a composition according to claim 1 or 7 to said patient.
